(19) United States
(12) Patent Application Publication
(10) Pub. No.: US 2016/0272706 A1
(43) Pub. Date: Sep. 22, 2016

(54) IL-13 BINDING PROTEINS AND USES THEREOF

(71) Applicant: MEDIMMUNE LIMITED, Cambridge (GB)

(72) Inventors: Sara Carmen, Cambridge (GB); David Lowe, Cambridge (GB); Matthew J. Gardener, Cambridge (GB); D. Gareth Rees, Cambridge (GB); Ian Strickland, Cambridge (GB); Richard D. May, Cambridge (GB); Tarik Senussi, Cambridge (GB); Bojana Popovic, Cambridge (GB); Jianqing Xu, Cambridge (GB); Suzanne J. Gibson, Cambridge (GB); Diane Hatton, Cambridge (GB)

(21) Appl. No.: 14/992,281

(22) Filed: Jan. 11, 2016

Related U.S. Application Data
(60) Provisional application No. 62/102,305, filed on Jan. 12, 2015.

(51) Int. Cl. C07K 16/24; C07K 16/28; A61K 51/10

(52) U.S. Cl. CPC C07K 16/244; A61K 51/1021; C07K 16/2866; C07K 2317/56; C07K 2317/565; C07K 2317/567; C07K 2317/76; C07K 2317/92; C07K 2317/21; C07K 2317/24; C07K 2317/31; C07K 2317/55; C07K 2317/54; C07K 2317/626; C07K 2317/622; C07K 2317/64; C07K 2317/52; A61K 2039/505

(57) ABSTRACT

Novel anti-IL-13 antigen-binding proteins such as antibodies and antigen-binding fragments thereof are provided. Methods of using the proteins to reduce IL-13 activity and to treat IL-13-associated diseases and conditions are further provided.

### REFERENCE TO SEQUENCE LISTING SUBMITTED ELECTRONICALLY

The content of the electronically submitted sequence listing in ASCII text file (IL13NG-100US1_SL.txt; Size: 216,752 bytes; and Date of Creation: Jan. 11, 2016) filed with the application is incorporated herein by reference in its entirety.

### FIELD OF INVENTION

The present invention relates to antigen-binding proteins for IL-13, in particular human IL-13 and in particular anti-IL-13 antibody molecules and antigen-binding fragments thereof, e.g. those which neutralise IL-13 activity. It further relates to methods for using anti-IL-13 antibody molecules and antigen-binding fragments thereof in diagnosis or treatment of IL-13 related diseases or conditions, including asthma, chronic obstructive pulmonary disease (COPD), idiopathic pulmonary fibrosis (IPF), atopic dermatitis, allergic rhinitis, fibrosis, scleroderma, systemic sclerosis, pulmonary fibrosis, liver fibrosis, inflammatory bowel disease, ulcerative colitis, Sjögren's Syndrome, and Hodgkin's lymphoma.

The antigen-binding proteins of the disclosure are derived from the BAK1183H4 antibody by light chain randomisation and are thus of the BAK1183H4 lineage. However, they have improved affinity for human IL-13 compared to BAK1183H4 while still retaining low aggregation and high stability as a result of mutations in their light chain complementarity determining regions (LCDRs) and/or framework regions.

Further aspects of the present disclosure provide for compositions containing antigen-binding proteins of the disclosure, and their use in methods of inhibiting or neutralising IL-13, including methods of treatment of the human or animal body by therapy.

The present disclosure provides antibody molecules and antigen-binding fragments thereof that bind and neutralise IL-13, which are thus of use in any of a variety of therapeutic treatments, as indicated by the experimentation contained herein and further by the supporting technical literature.

### BACKGROUND TO THE INVENTION

Interleukin (IL)-13 is a 114 amino acid cytokine with an unmodified molecular mass of approximately 12 kDa [1,2]. IL-13 is most closely related by sequence to IL-4 with which it shares 30% sequence similarity at the amino acid level. The human IL-13 gene is located on chromosome 5q31 adjacent to the IL-4 gene [1][2]. This region of chromosome 5q contains gene sequences for other Th2 lymphocyte derived cytokines including GM-CSF and IL-5, whose levels together with IL-4 have been shown to correlate with disease severity in asthmatics and rodent models of allergic inflammation [3][4][5][6][7][8].

Although initially identified as a Th2 CD4+ lymphocyte derived cytokine, IL-13 is also produced by Th1 CD4+ T-cells, CD8+ T lymphocytes NK cells, and non-T-cell populations such as mast cells, basophils, eosinophils, macrophages, monocytes, and airway smooth muscle cells.

IL-13 is reported to mediate its effects through a receptor system that includes the IL-4 receptor α chain (IL-4Rα), which itself can bind IL-4 but not IL-13, and at least two other cell surface proteins, IL-13Rα1 and IL-13Rα2 [9][10]. IL-13Rα1 can bind IL-13 with low affinity, subsequently recruiting IL-4Rα to form a high affinity functional receptor that signals [11][12]. The Genbank database lists the amino acid sequence and the nucleic acid sequence of IL-13Rα1 as NP_001551 and Y10659 respectively. Studies in STAT6 (signal transducer and activator of transcription 6)-deficient mice have revealed that IL-13, in a manner similar to IL-4, signals by utilising the JAK-STAT6 pathway [13][14]. IL-13Rα2 shares 37% sequence identity with IL-13Rα1 at the amino acid level and binds IL-13 with high affinity [15][16]. However, IL-13Rα2 has a shorter cytoplasmic tail that lacks known signaling motifs. Cells expressing IL-13Rα2 are not responsive to IL-13 even in the presence of IL-4Rα [17]. It is postulated, therefore, that IL-13Rα2 acts as a decoy receptor regulating IL-13 but not IL-4 function. This is supported by studies in IL-13Rα2-deficient mice whose phenotype was consistent with increased responsiveness to IL-13 [18][19]. The Genbank database lists the amino acid sequence and the nucleic acid sequence of IL-13Rα2 as NP_000631 and Y08768 respectively.

The signalling IL-13Rα1/IL-4Rα receptor complex is expressed on human B-cells, mast cells, monocyte/macrophages, dendritic cells, eosinophils, basophils, fibroblasts, endothelial cells, airway epithelial cells, and airway smooth muscle cells.

Bronchial asthma is a common persistent inflammatory disease of the lung characterised by airways hyper-responsiveness, mucus overproduction, fibrosis, and raised serum IgE levels. Airways hyper-responsiveness (AHR) is the exaggerated constriction of the airways to non-specific stimuli such as cold air. Both AHR and mucus overproduction are thought to be responsible for the variable airway obstruction that leads to the shortness of breath characteristic of asthma attacks (exacerbations) and which is responsible for the mortality associated with this disease (around 2000 deaths/year in the United Kingdom; around 250,000 annual deaths worldwide. See Clinical Respiratory Medicine. Eds Richard K. Albert, Stephen G. Spiro, James R. Jett. Elsevier Health Sciences, 2008 at page 554).

The incidence of asthma, along with other allergic diseases, has increased significantly in recent years [20][21]. For example, currently, around 10% of the population of the United Kingdom (UK) has been diagnosed as asthmatic.

Current British Thoracic Society (BTS) and Global Initiative for Asthma (GINA) guidelines suggest a stepwise approach to the treatment of asthma [22, 23]. Mild to moderate asthma can usually be controlled by the use of inhaled corticosteroids, in combination with beta-agonists or leukotriene inhibitors. However, due to the documented side effects of corticosteroids, patients tend not to comply with the treatment regime which reduces the effectiveness of treatment [24-26].

There is a clear need for new treatments for subjects with more severe disease, who often gain very limited benefit from either higher doses of inhaled or oral corticosteroids recommended by asthma guidelines. Long-term treatment with oral corticosteroids is associated with side effects such as osteoporosis, slowed growth rates in children, diabetes, and oral candidiasis [66]. As both beneficial and adverse effects of corticosteroids are mediated via the same receptor, treatment is a balance between safety and efficacy. Hospitalisation of these patients, who represent around 6% of the UK asthma population, as a result of severe exacerbations accounts for the majority of the significant economic burden of asthma on healthcare authorities [67].

It is believed that the pathology of asthma is caused by ongoing Th2 lymphocyte-mediated inflammation that results from inappropriate responses of the immune system to harmless antigens. Evidence has been accrued which implicates IL-13, rather than the classical Th2-derived cytokine IL-4, as the key mediator in the pathogenesis of established airway disease.

Administration of recombinant IL-13 to the airways of naïve non-sensitised rodents caused many aspects of the asthma phenotype, including airway inflammation, mucus production and AHR to increase [27][28][29][30]. A similar phenotype was observed in a transgenic mouse in which IL-13 was specifically overexpressed in the lung. In this model, more chronic exposure to IL-13 also resulted in fibrosis [31].

Further, in rodent models of allergic disease many aspects of the asthma phenotype have been associated with IL-13. Soluble murine IL-13Rα2, a potent IL-13 neutraliser, has been shown to inhibit AHR, mucus hypersecretion, and the influx of inflammatory cells which are characteristics of this rodent model [27][28][30]. In complementary studies, mice in which the IL-13 gene had been deleted failed to develop allergen-induced AHR. AHR could be restored in these IL-13-deficient mice by the administration of recombinant IL-13. In contrast, IL-4-deficient mice developed airway disease in this model [32][33].

Using a longer-term allergen-induced pulmonary inflammation model, Taube at al. demonstrated the efficacy of soluble murine IL-13Rα2 against established airway disease [34]. Soluble murine IL-13Rα2 inhibited AHR, mucus overproduction, and to a lesser extent airway inflammation. In contrast, soluble IL-4Rα, which binds and antagonises IL-4, had little effect on AHR or airway inflammation in this system [35]. These findings were supported by Blease et al. who developed a chronic fungal model of asthma in which polyclonal antibodies against IL-13 but not IL-4 were able to reduce mucus overproduction, AHR, and subepithelial fibrosis [36].

A number of genetic polymorphisms in the IL-13 gene have also been linked to allergic disease. In particular, a variant of the IL-13 gene in which the arginine residue at amino acid 130 is substituted with glutamine (Q130R) has been associated with bronchial asthma, atopic dermatitis, and raised serum IgE levels [37][38][39][40]. This particular IL-13 variant is also referred to as the Q110R variant (arginine residue at amino acid 110 is substituted with glutamine) by some groups who exclude the 20 amino acid signal sequence from the amino acid count. Arima et al, [41] report that this variant is associated with raised levels of IL-13 in serum. The IL-13 variant (Q130R) and antibodies to this variant are discussed in WO 01/62933. An IL-13 promoter polymorphism, which alters IL-13 production, has also been associated with allergic asthma [42].

Raised levels of IL-13 have also been measured in human subjects with asthma, atopic rhinitis (hay fever), allergic dermatitis (eczema), and chronic sinusitis. For example levels of IL-13 were found to be higher in bronchial biopsies, sputum, and broncho-alveolar lavage (BAL) cells from asthmatics compared to control subjects [43][44][45][46]. Further, levels of IL-13 in BAL samples increased in asthmatic individuals upon challenge with allergen [47][48]. The IL-13 production capacity of CD4(+) T cells has further been shown to be useful marker of risk for subsequent development of allergic disease in newborns [49].

Li et al [75] have reported the effects of a neutralising anti-mouse IL-13 antibody in a chronic mouse model of asthma. Chronic asthma-like response (such as AHR, severe airway inflammation, hyper mucus productions) was induced in OVA sensitised mice. Li et al report that administration of an IL-13 antibody at the time of each OVA challenge suppresses AHR, eosinophil infiltration, serum IgE levels, proinflammatory cytokine/chemokine levels, and airway remodelling [14].

IL-13 may play a role in the pathogenesis of inflammatory bowel disease. Heller et al. [78] report that neutralisation of IL-13 by administration of soluble IL-13Rα2 ameliorated colonic inflammation in a murine model of human ulcerative colitis [78]. Correspondingly, IL-13 expression was higher in rectal biopsy specimens from ulcerative colitis patients when compared to controls [77].

Aside from asthma, IL-13 has been associated with other fibrotic conditions. Increased levels of IL-13, up to a 1000 fold higher than IL-4, have been measured in the serum of patients with systemic sclerosis [50] and in BAL samples from patients affected with other forms of pulmonary fibrosis [51]. Correspondingly, overexpression of IL-13 but not IL-4 in the mouse lung resulted in pronounced fibrosis [52][53]. The contribution of IL-13 to fibrosis in tissues other than the lung has been extensively studied in a mouse model of parasite-induced liver fibrosis. Specific inhibition of IL-13 by administration of soluble IL-13Rα2 or IL-13 gene disruption, but not ablation of IL-4 production prevented fibrogenesis in the liver [54][55][56].

Chronic Obstructive Pulmonary Disease (COPD) includes patient populations with varying degrees of chronic bronchitis, small airway disease and emphysema and is characterised by progressive irreversible lung function decline that responds poorly to current asthma based therapy [68].

The incidence of COPD has risen dramatically in recent years to become the fourth leading cause of death worldwide (World Health Organisation). COPD therefore represents a large unmet medical need.

The underlying causes of COPD remain poorly understood. The “Dutch hypothesis” proposes that there is a common susceptibility to COPD and asthma and therefore, that similar mechanisms may contribute to the pathogenesis of both disorders [57].

Zheng et al [58] have demonstrated that overexpression of IL-13 in the mouse lung caused emphysema, elevated mucus production, and inflammation, reflecting aspects of human COPD. Furthermore, AHR, an IL-13 dependent response in murine models of allergic inflammation, has been shown to be predictive of lung function decline in smokers [59]. A link has also been established between an IL-13 promoter polymorphism and susceptibility to develop COPD [60].

The signs are therefore that IL-13 plays an important role in the pathogenesis of COPD, particularly in patients with asthma-like features including AHR and eosinophilia. mRNA levels of IL-13 have been shown to be higher in autopsy tissue samples from subjects with a history of COPD when compared to lung samples from subjects with no reported lung disease (J. Elias, Oral communication at American Thoracic Society Annual Meeting 2002). In another study, raised levels of IL-13 were demonstrated by immunohistochemistry in peripheral lung sections from COPD patients [69].

Hodgkin's disease is a common type of lymphoma, which accounts for approximately 7,500 cases per year in the United States. Hodgkin's disease is unusual among malignancies in that the neoplastic Reed-Sternberg cell, often derived from B-cells, make up only a small proportion of the clinically detectable mass. Hodgkin's disease-derived cell lines and primary Reed-Sternberg cells frequently express IL-13 and its receptor [61]. As IL-13 promotes cell survival and proliferation in normal B-cells, it was proposed that IL-13 could act as a growth factor for Reed-Sternberg cells. Skinnider et al. have demonstrated that neutralising antibodies against IL-13 can inhibit the growth of Hodgkin's disease-derived cell lines in vitro [62]. This finding suggested that Reed-Sternberg cells might enhance their own survival by an IL-13 autocrine and paracrine cytokine loop. Consistent with this hypothesis, raised levels of IL-13 have been detected in the serum of some Hodgkin's disease patients when compared to normal controls [63]. IL-13 inhibitors may therefore prevent disease progression by inhibiting proliferation of malignant Reed-Sternberg cells.

Many human cancer cells express immunogenic tumour specific antigens. However, although many tumours spontaneously regress, a number evade the immune system (immunosurveillance) by suppressing T-cell-mediated immunity. Terabe et al. [64] have demonstrated a role of IL-13 in immunosuppression in a mouse model in which tumours spontaneously regress after initial growth and then recur. Specific inhibition of IL-13, with soluble IL-13Rα2, protected these mice from tumour recurrence. Terabe et al [64] went on to show that IL-13 suppresses the differentiation of tumour specific CD8+ cytotoxic lymphocytes that mediate anti-tumour immune responses.

IL-13 inhibitors may, therefore, be used therapeutically to prevent tumour recurrence or metastasis. Inhibition of IL-13 has been shown to enhance anti-viral vaccines in animal models and may be beneficial in the treatment of HIV and other infectious diseases [65].

It should be noted that generally herein reference to interleukin-13 or IL-13 is, except where context dictates otherwise, reference to human IL-13. This is also referred to in places as “the antigen.”

Antibody molecules that bind human IL-13 are described in WO 2005/007699 and U.S. Pat. No. 7,829,090 (each herein incorporated by reference in its entirety), including the BAK1183H4 antibody (from which the antigen-binding proteins of the disclosure are derived). However, there remains a need for improved anti-IL-13 antibodies having higher affinity and increased serum persistence or half-life to increase efficacy and reduce frequency of administration and increase patient compliance.

### BRIEF SUMMARY OF THE INVENTION

The present disclosure provides antigen-binding proteins derived from the BAK1183H4 antibody by light chain randomisation that bind to human IL-13 with a better affinity than the BAK1183H4 due to substitutions in their light chain CDR (LCDR) sequences and/or optionally one or more further substitutions in framework regions. As is well understood in the art, increasing the affinity of anti-IL-13 antigen-binding proteins sometimes results in high aggregation rates which can reduce efficacy or thermal stability. However, the optimized antigen-binding proteins of the disclosure have increased affinity compared to BAK1183H4 with aggregation comparable to BAK1183H4.

Instability of IgG domains can correlate with unfavorable Chemistry, Manufacturing, and Control (CMC) properties such as decreased thermal stability and solubility, increased aggregation or fragmentation ultimately leading to increased purity loss, limited formulation/delivery options, and other developability challenges. Thermal instability of immunoglobulins is sometimes observed when IgG1 constant domains are engineered to reduce effector function and/or increase serum half-life. See, e.g., PCT/US2013/036872 filed Apr. 17, 2013, published as WO2013165690, herein incorporated by reference it its entirety.

For example, Dall'Acqua et al. (2006, J. Biol. Chem.; 281:23514-24) described an IgG1 antibody whose Fc region was mutated at position 252, 254, and 256 (M252Y/S254T/T256E EU numbering, (Kabat, E. A., Wu, T. T., Perry, H. M., Gottesman, K. S., and Foeller. (1991) Sequences of Proteins of Immunological Interest, U.S. Public Health Service, National Institutes of Health, Washington, D.C., hereinafter “YTE”). These mutations increase the binding to human FcRn by about 10-fold at pH 6.0 while allowing efficient release at pH 7.4 significantly increasing serum half-life in cynomolgus monkey as compared to wild-type IgG1. See Dall'Acqua et al, 2002, J Immunol.; 169:5171-80.

When an IgG1 constant domain containing the YTE set of mutations was incorporated into the antigen-binding proteins of the present disclosure, thermal stability of the antigen-binding proteins of the present disclosure was surprisingly comparable to BAK1183H4. Thus, the antigen-binding proteins of the present disclosure not only have increased affinity to IL-13 with low aggregation, but also have comparable thermal stability to the BAK1183H4 parent due to substitutions in their light chain CDR (LCDR) sequences and/or optionally one or more further substitutions in framework regions.

The present disclosure provides an isolated antigen-binding protein or antigen-binding fragment thereof that binds human IL-13, comprising an antigen-binding site composed of a variable heavy (VH) domain and a variable light (VL) domain, wherein the VH domain comprises HCDR1, HCDR2, and HCDR3 and the VL domain comprises LCDR1, LCDR2, and LCDR3, and wherein:

HCDR1 comprises the amino acid sequence of SEQ ID NO: 13;
HCDR2 comprises the amino acid sequence of SEQ ID NO: 14;
HCDR3 comprises the amino acid sequence of SEQ ID NO: 15;
LCDR1 comprises the amino acid sequence having the formula:

GGNLX1LX2LX3LX4LX5LVH

wherein LX1 is selected from the group consisting of L and M,
LX2 is selected from the group consisting of L, I and V,
LX3 is selected from the group consisting of G and A,
LX4 is selected from the group consisting of S and A, and
LX5 is selected from the group consisting of R and Y; (SEQ ID NO: 251)
LCDR2 comprises the amino acid sequence having the formula:

DDLX6DRPS

wherein LX6 is selected from the group consisting of G, I, E, M and Q; (SEQ ID NO:252) and

LCDR3 comprises the amino acid sequence having the formula:

QVWDTGSLX7PVV

wherein LX7 is selected from the group consisting of D, R, L and S (SEQ ID NO:253).

In some embodiments, the antigen-binding protein of the disclosure comprises a set of CDRs: HCDR1, HCDR2, HCDR3, LCDR1, LCDR2 and LCDR3, as shown in any one of Tables 3-6 below.

The disclosure also provides an isolated antigen-binding protein or an antigen-binding fragment thereof that binds human IL-13 comprising an antigen-binding site composed of a variable heavy (VH) domain and a variable light (VL) domain comprising a set of CDRs: HCDR1, HCDR2, HCDR3, LCDR1, LCDR2 and LCDR3, wherein the set of CDRs is selected from the group consisting of:
(a) HCDR1 comprises the amino acid sequence shown as SEQ ID NO: 13, HCDR2 comprises the amino acid sequence as SEQ ID NO: 14, HCDR3 comprises the amino acid sequence as SEQ ID NO: 15, LCDR1 comprises the amino acid sequence shown as SEQ ID NO: 18, LCDR2 comprises the amino acid sequence shown as SEQ ID NO: 19, and LCDR3 comprises the amino acid sequence shown as SEQ ID NO: 20;
(b) HCDR1 comprises the amino acid sequence shown as SEQ ID NO: 23, HCDR2 comprises the amino acid sequence as SEQ ID NO: 24, HCDR3 comprises the amino acid sequence as SEQ ID NO: 25, LCDR1 comprises the amino acid sequence shown as SEQ ID NO: 28, LCDR2 comprises the amino acid sequence shown as SEQ ID NO: 29, and LCDR3 comprises the amino acid sequence shown as SEQ ID NO: 30; and
(c) HCDR1 comprises the amino acid sequence shown as SEQ ID NO: 33, HCDR2 comprises the amino acid sequence shown as SEQ ID NO: 34, HCDR3 comprises the amino acid sequence shown as SEQ ID NO: 35, LCDR1 comprises the amino acid sequence shown as SEQ ID NO: 38, LCDR2 comprises the amino acid sequence shown as SEQ ID NO: 38, and LCDR3 comprises the amino acid sequence shown as SEQ ID NO: 40.

The disclosure further provides an isolated antigen-binding protein or antigen-binding fragment thereof that binds human IL-13, comprising a VH domain and a VL domain selected from the group consisting of:

(a) a VH domain comprising SEQ ID NO: 12 and a VL domain comprising SEQ ID NO: 17 (13NG0083);
(b) a VH domain comprising SEQ ID NO: 22 and a VL domain comprising SEQ ID NO: 27 (13NG0073);
(c) a VH domain comprising SEQ ID NO: 32 and a VL domain comprising SEQ ID NO: 37 (13NG0074);
(d) a VH domain comprising SEQ ID NO: 112 and a VL domain comprising SEQ ID NO: 117 (13NG0071);
(e) a VH domain comprising SEQ ID NO: 42 and a VL domain comprising SEQ ID NO: 47 (13NG0068);
(f) a VH domain comprising SEQ ID NO: 52 and a VL domain comprising SEQ ID NO: 57 (13NG0067);
(g) a VH domain comprising SEQ ID NO: 62 and a VL domain comprising SEQ ID NO: 67 (13NG0069);
(h) a VH domain comprising SEQ ID NO: 72 and a VL domain comprising SEQ ID NO: 77 (13NG0076);
(i) a VH domain comprising SEQ ID NO: 82 and a VL domain comprising SEQ ID NO: 87 (13NG0070);
(j) a VH domain comprising SEQ ID NO: 92 and a VL domain comprising SEQ ID NO: 97 (13NG0075);
(k) a VH domain comprising SEQ ID NO: 102 and a VL domain comprising SEQ ID NO: 107 (13NG0077);
(l) a VH domain comprising SEQ ID NO: 122 and a VL domain comprising SEQ ID NO: 127 (13NG0072);
(m) a VH domain comprising SEQ ID NO: 242 and a VL domain comprising SEQ ID NO: 247 (13NG0025);
(n) a VH domain comprising SEQ ID NO: 222 and a VL domain comprising SEQ ID NO: 227 (13NG0078);
(o) a VH domain comprising SEQ ID NO: 142 and a VL domain comprising SEQ ID NO: 147 (13NG0079);
(p) a VH domain comprising SEQ ID NO: 152 and a VL domain comprising SEQ ID NO: 157 (13NG0080);
(q) a VH domain comprising SEQ ID NO: 132 and a VL domain comprising SEQ ID NO: 137 (13NG0081);
(r) a VH domain comprising SEQ ID NO: 192 and a VL domain comprising SEQ ID NO: 197 (13NG0082);
(s) a VH domain comprising SEQ ID NO: 182 and a VL domain comprising SEQ ID NO: 187 (13NG0084);
(t) a VH domain comprising SEQ ID NO: 212 and a VL domain comprising SEQ ID NO: 217 (13NG0085);
(u) a VH domain comprising SEQ ID NO: 162 and a VL domain comprising SEQ ID NO: 167 (13NG0086);
(v) a VH domain comprising SEQ ID NO: 202 and a VL domain comprising SEQ ID NO: 207 (13NG0087); and
(w) a VH domain comprising SEQ ID NO: 172 and a VL domain comprising SEQ ID NO: 177 (13NG0088).

In one embodiment, the antigen-binding protein of the disclosure, or antigen-binding fragment thereof, comprises a set of CDRs: HCDR1, HCDR2, HCDR3, LCDR1, LCDR2 and LCDR3, wherein:

HCDR1 comprises the amino acid sequence shown as SEQ ID NO: 233,
HCDR2 comprises the amino acid sequence shown as SEQ ID NO: 234,
HCDR3 comprises the amino acid sequence shown as SEQ ID NO: 235,
LCDR1 comprises the amino acid sequence shown as SEQ ID NO: 238,
LCDR2 comprises the amino acid sequence shown as SEQ ID NO: 239, and
LCDR3 comprises the amino acid sequence shown as SEQ ID NO: 240 (i.e. clone 13NG0027).

In one embodiment, the antigen-binding protein of the disclosure, or fragment thereof, comprises a VH domain comprising SEQ ID NO: 232 and a VL domain comprising SEQ ID NO: 237 (i.e. clone 13NG0027).

The antigen-binding protein of the disclosure may have one or more properties selected from the group consisting of:
(a) Competes with a BAK1183H4 antibody for binding to IL-13, wherein the BAK1183H4 antibody comprises a VH domain comprising the amino acid sequence of SEQ ID NO: 2 and a VL domain comprising the amino acid sequence of SEQ ID NO: 7;
(b) Binds human IL-13 with an affinity better than that of the BAK1183H4 antibody, wherein the BAK1183H4 antibody comprises a VH domain comprising the amino acid sequence of SEQ ID NO: 2 and a VL domain comprising the amino acid sequence of SEQ ID NO: 7; and
(c) Binds human IL-13 with a KD value of less than about 80 pM, less than about 50 pM, less than about 20 pM, or less than about 10 pM.

In additional embodiments, the antigen-binding protein of the disclosure comprises a human IgG1 constant domain and a human lambda constant domain.

The antigen-binding protein of the disclosure may also comprise an IgG1 Fc domain containing a mutation of M252Y, S254T, and T256E, wherein the position numbering is according to the EU index as in Kabat.

The disclosure further provides the antigen-binding protein of the disclosure for use in a method of treatment of a disease or condition selected from the group consisting of asthma, chronic obstructive pulmonary disease (COPD), idiopathic pulmonary fibrosis (IPF), atopic dermatitis, allergic rhinitis, fibrosis, scleroderma, systemic sclerosis, pulmonary fibrosis, liver fibrosis, inflammatory bowel disease, ulcerative colitis, Sjögren's Syndrome, and Hodgkin's lymphoma.

The disclosure further provides antigen-binding proteins comprising the VH, VL, and CDR sequences provided in FIGS. 1-4 and antigen-binding proteins with the features demonstrated in Examples 1-11 and FIGS. 5-19.

These antigen-binding proteins and other aspects of the disclosure are described in more detail below.

### BRIEF DESCRIPTION OF THE DRAWINGS

FIG. 1 shows the VL sequences of 22 variants from the mini-recombination library identified as hits in the biochemical assay. CDR regions are in boxes. Differences in amino acid sequence compared to parent (BAK1183H04) are highlighted in grey. Vernier residues are denoted with a black bar at the top of the sequence alignment. FIG. 1A shows the VL sequences (SEQ ID NOS 300-335, respectively, in order of appearance) of the first panel of purified scFvs from the mini-library screened in the biochemical assay. FIG. 1B shows the VL sequence alignments (SEQ ID NOS 336-371, respectively, in order of appearance) of the second panel of purified scFvs from the mini-library screened in the biochemical assay. All variants show 3-7 fold improvements in IC50 compared to parent (Table in FIGS. 1a and 1b).

FIG. 2 shows a sequence alignment of clones identified from the mini-recombination library. Differences in amino acid sequence compared to parent (BAK1183H04) are highlighted in grey. Vernier residues are denoted with a black bar at the top of the sequence alignment. FIG. 2A shows the heavy chain sequence alignments (SEQ ID NOS 372-396, respectively, in order of appearance), and FIG. 2B shows the light chain sequence alignments (SEQ ID NOS 397-421, respectively, in order of appearance).

FIG. 3 shows a sequence alignment of the three clones 13NG0073, 13NG0074, and 13NG0083 identified from the mini-library recombination strategy that were taken forward for further characterisation. Differences in amino acid sequence compared to parent (1183H04) are highlighted in grey. Vernier residues are denoted with a black bar at the top of the sequence alignment. FIG. 3 discloses SEQ ID NOS 422-429, respectively, in order of appearance.

FIG. 4 shows a sequence alignment of two variants from the pre-recombination selections. The VL CDR1 and VL CDR2 of clone 13NG0025 and 13NG0027 respectively, when recombined with a final variant that had the single amino acid substitution from D to S at position 95a in the VL CDR3, resulted in the VL sequence of clone 13NG0083. Differences in amino acid sequence compared to parent (BAK1183H04) are highlighted in grey. Vernier residues are denoted with a black bar at the top of the sequence alignment. The individual fold improvements observed for these two clones were modest; yet recombining them (with the additional mutation in the VL CDR3) resulted in an unexpected, 5.2-fold improvement in affinity. FIG. 4 discloses SEQ ID NOS 430-433, respectively, in order of appearance.

FIG. 5 shows the potency of the 13NG0083 clone in a TF1 proliferation assay. Squares represent the results for an isotype control, and circles represent the results for 13NG0083 (IgG format with a YTE mutation in the Fc region). 13NG0083 potently inhibits TF1 proliferation (mean IC50=165 pM (95% confidence interval=26-1052 pM)). CPM: counts per minute. A representative experiment is shown; data is arithmetic mean of duplicate values±SEM.

FIG. 6 shows the IC₅₀ values for 13NG0083 variants derived from a receptor-ligand competition assay. Data are shown as geometric mean±95% confidence intervals. Parent IgG1-YTE (1183H4_VH_VL_nonGL IgG1-YTE) and human IL-13 Receptorα2 are included for reference. The 13NG0083 variants show a significant improvement in mean potency from the parent (1183H4_VH_VL_nonGL IgG1-YTE; IC₅₀=1.34 nM) with little effect seen with altering the IgG format (13NG0083 IgG1-YTE; IC₅₀=423 pM; vs 13NG0083 ngl-2 IgG4-P-YTE; IC₅₀=496 pM), nor upon changes to germline (13NG0083 fgl Human IgG1-YTE; IC₅₀=734 pM; vs. 13NG0083 fgl human IgG4-P-YTE; IC₅₀=622 pM). Symbols represent individual experiment repeats. IgG4-P: IgG4 S241P.

FIG. 7 shows the affinity (K_{D}) and 95% confidence intervals (C.I.) of the IL-13NG clones, 13NG0073 (“73”) (KD of 4.6 pM), 13NG0074 (“74”) (KD of 4.0 pM), and 13NG0083 (“83”) (KD of 6.0 pM).

FIG. 8 shows the results of in vitro testing of R130 (circles), Q130 (squares) and Q105 (triangles) human IL-13 variants in a TF1 proliferation assay. A representative experiment is shown, and data is arithmetic mean of duplicate values±SEM. CPM: counts per minute.

FIG. 9 shows inhibition of the IL-13 variant Q105 by 13NG0083 in a TF1 potency assay. Squares represent the results for an isotype control, and circles represent the results for fully germlined (FGL) 13NG0083 (IgG format with a YTE mutation in the Fc region). 13NG0083 (“IL13NG_FGL IgG1 YTE) inhibits the IL-13 Q105 variant. CPM: counts per minute. A representative experiment is shown, and data is arithmetic mean of duplicate values±SEM.

FIG. 10 shows the IC50 values for 13NG0083 variants (including 13NG0083 human IgG1+YTE (“hIgG1-YTE”) and 13NG0083 human IgG4-P (IgG4 S241P)+YTE (“IgG4-P-YTE” or “hIgG4-P-YTE”); either fully germlined (“fgl”) or non-germlined (“ngl2”)) in a receptor-ligand competition assay using the variant forms of IL-13: Q105 (FIG. 10A), Q130R (FIG. 10B) and Cynomolgus IL-13 (FIG. 10C). The common IL-13 variant R130 is included as a standard in FIGS. 10A and B.

FIG. 11 shows the functional species cross-reactivity of 13NG0083 with human (FIG. 11A), cynomolgus (FIG. 11B), and mouse (FIG. 11C) IL-13. Squares represent the results for an isotype control, and circles represent the results for fully germlined (FGL) 13NG0083 (IgG format with a YTE mutation in the Fc region). Both human and cynomolgus IL-13 were inhibited by 13NG0083. Mouse IL-13 supported TF1 proliferation; however no inhibition was observed with 13NG0083 except a small reduction at the highest concentration of the antibody. CPM: counts per minute. A representative experiment is shown, and data is the arithmetic mean of duplicate values±SEM.

FIG. 12 shows binding of human and cynomolgus FcRn to 13NG0083. The table shows binding affinity (KD/nM) of 13NG0083 (“IL13NG_83” in an IgG1 format with a YTE mutation in the Fc region) or NIP228, the isotype control, to human or cynomolgus FcRn as measured by surface plasmon resonance (Biacore). The binding affinity of both antibodies to each FcRn species at pH 7.4 is also expressed as a percentage of the binding affinity at pH 6 (“pH7.6/pH6”). 13NG0083-IgG1-YTE bound both human and cyno FcRns with a high affinity (KD of 153 and 205, respectively).

FIG. 13 shows the stability of 13NG0073 (squares; “IL13NG0073”) and 13NG0083 (circles; “IL13NG0083”) incubated in human whole blood. Antibodies IL13NG0083 and 11130073 were incubated in human whole blood for either 0 (FIG. 13A) or 24 hours (FIG. 13B) and then titrated into a TF1 proliferation assay. Both 13NG0073 and 13NG0083 were stable after incubating in serum for 24 hours as each effectively inhibited TF1 cell proliferation.

FIG. 14 shows the relative expression titre of various combinations of 13NG0083 heavy chain (Hc) and light chains (Lc) expressed in CHO cells. Substituting the 13NG0083 light chain with other light chains from different antibodies improved expression. Mg/L=milligrams per liter.

FIG. 15 shows the relative expression titre of nine 13NG0083 light chain mutants expressed in CHO cells compared to unmodified 13NG0083 light chain (“Lc”) or a control antibody (“Hc&Lc3”). Two mutants M27I and E52G demonstrated a consistent improvement in expression compared to unmodified 13NG0083. Mg/L=milligrams per liter.

FIG. 16 shows the 13NG0083 light chain structural model. Assessment of the light chain sequence/structure using this structural model identified a strong hydrophilic and negative-charged region on the tip of CDR2 (50-DDED-53 (SEQ ID NO: 286)). Review of ˜1045 antibody structures available in the pdb database (up to 2013) showed that this sequence motif (4 consecutive negative amino acids (“----”) was never observed, while the relative abundance of several other amino acid motifs in the antibody structures available in the pdb database is reported. FIG. 16 discloses SEQ ID NOS 290-297, 288, 298 and 299, respectively, in order of appearance.

FIG. 17 shows the relative expression titre of 13NG0083 mutants expressed in CHO cells compared to unmodified 13NG0083 light chain (“Lc”) and control antibodies (“Hc&Lc3” or “Control Ab 6”). Significant improvement in expression compared to unmodified 13NG0083 was observed when combining M27I+E52G or combining M27I+E52N. All supercharge reversion mutants (D51N (DNED (SEQ ID NO: 287)); E52N (DDND (SEQ ID NO: 288)); and D53N (DDEN (SEQ ID NO: 289))) showed improved expression compared to unmodified 13NG0083. Mg/L=milligrams per liter.

FIGS. 18A and 18B show the results of an ELISA assay to assess binding of 13NG0083 light chain mutants described in FIGS. 15 and 17 to IL-13. Of note, mutant (DNED (SEQ ID NO: 287)) lost binding to IL-13. “WT Ph2” denotes wildtype 13NG0083.

FIGS. 19A and 19B show inhibition by various 13NG0083 light chain mutants described in FIGS. 15 and 17 in a TF1 potency assay compared to control antibodies (“Hc&Lc3” or “Control Ab6”). Mutant DNED (SEQ ID NO: 287) did not inhibit proliferation of TF-1 cells. However, all of the mutants tested, including mutant DDEN (SEQ ID NO: 289), bound and inhibited IL-13-induced proliferation of TF-1 cells with a similar potency as unmodified 13NG0083 (“WT”). FIG. 19 B discloses “DDND” as SEQ ID NO: 288.

FIG. 20 shows the amplified light chain CDR2 structural models of 13NG0083 (left) and the 13NG0083 light chain 50-DDEN-53 (SEQ ID NO: 289) mutant (right), obtained from each individual molecular dynamics simulation. The comparison between these two systems suggested that the D53N substitution could effectively relieve local charge pressure, and allow the side chain of N53 to form hydrogen bonds with its neighboring residues to improve the CDR2 stability. FIG. 20 discloses “DDED” as SEQ ID NO: 286.

### DETAILED DESCRIPTION

In various aspects and embodiments of the disclosure there is provided the subject-matter described below. Any aspect or embodiment described herein can be combined with any other aspect of embodiment described herein.

### Definitions

The terms “a,” “an,” and “the” include plural referents unless the context clearly dictates otherwise. For example, “an antigen-binding protein” is understood to represent one or more antigen-binding proteins. The terms “a” (or “an”), as well as the terms “one or more,” and “at least one” can be used interchangeably herein. Furthermore, “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

The term “comprise” is generally used in the sense of include, that is to say permitting the presence of one or more features or components. Wherever aspects are described herein with the language “comprising,” otherwise analogous aspects described in terms of “consisting of,” and/or “consisting essentially of” are also provided.

The term “about” as used in connection with a numerical value throughout the specification and the claims denotes an interval of accuracy, familiar and acceptable to a person skilled in the art. In general, such interval of accuracy is ±10%.

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary Of Biochemistry And Molecular Biology, Revised, 2000, Oxford University Press, provide one of skill with a general dictionary of many of the terms used in this disclosure.

Units, prefixes, and symbols are denoted in their Systéme International de Unites (SI) accepted form. Numeric ranges are inclusive of the numbers defining the range. Unless otherwise indicated, amino acid sequences are written left to right in amino to carboxy orientation. The headings provided herein are not limitations of the various aspects or aspects of the disclosure, which can be had by reference to the specification as a whole. Accordingly, the terms defined immediately below are more fully defined by reference to the specification in its entirety.

As used herein, the term “antibody” (or a fragment, variant, or derivative thereof) refers to at least the minimal portion of an antibody which is capable of binding to antigen, e.g., at least the variable domain of a heavy chain (VH) and the variable domain of a light chain (VL) in the context of a typical antibody produced by a B cell. Basic antibody structures in vertebrate systems are relatively well understood. See, e.g., Harlow et al., Antibodies: A Laboratory Manual, (Cold Spring Harbor Laboratory Press, 2nd ed. 1988).

Antibodies or antigen-binding fragments, variants, or derivatives thereof include, but are not limited to, polyclonal, monoclonal, human, humanized, or chimeric antibodies, single chain antibodies, epitope-binding fragments, e.g., Fab, Fab′ and F(ab′)2, Fd, Fvs, single-chain Fvs (scFv), single-chain antibodies, disulfide-linked Fvs (sdFv), fragments comprising either a VL or VH domain, fragments produced by a Fab expression library. ScFv molecules are known in the art and are described, e.g., in U.S. Pat. No. 5,892,019. Immunoglobulin or antibody molecules encompassed by this disclosure can be of any type (e.g., IgG, IgE, IgM, IgD, IgA, and IgY), class (e.g., IgG1, IgG2, IgG3, IgG4, IgA1 and IgA2) or subclass of immunoglobulin molecule.

The term “antibody molecule” describes an immunoglobulin whether natural or partly or wholly synthetically produced. The term also covers any polypeptide or protein comprising an antibody binding domain. Antibody fragments which comprise an antigen-binding domain are molecules such as Fab, scFv, Fv, dAb, Fd, and diabodies.

It is possible to take monoclonal and other antibodies and use techniques of recombinant DNA technology to produce other antibodies or chimeric molecules which retain the specificity of the original antibody. Such techniques can involve introducing DNA encoding the immunoglobulin variable region, or the complementarity determining regions (CDRs), of an antibody to the constant regions, or constant regions plus framework regions, of a different immunoglobulin. See, for instance, EP-A-184187, GB 2188638A, or EP-A-239400, and a large body of subsequent literature. A hybridoma or other cell producing an antibody can be subject to genetic mutation or other changes, which may or may not alter the binding specificity of antibodies produced.

As antibodies can be modified in a number of ways, the term “antibody molecule” should be construed as covering any antigen-binding protein or substance having an antibody antigen-binding domain with the required specificity. Thus, this term covers antibody fragments and derivatives, including any polypeptide comprising an immunoglobulin binding domain, whether natural or wholly or partially synthetic. Chimeric molecules comprising an immunoglobulin binding domain, or equivalent, fused to another polypeptide are therefore included. Cloning and expression of chimeric antibodies are described in EP-A-0120694 and EP-A-0125023, and a large body of subsequent literature.

Further techniques available in the art of antibody engineering have made it possible to isolate human and humanised antibodies. For example, human hybridomas can be made as described by Kontermann et al [70]. Phage display, another established technique for generating antigen-binding proteins has been described in detail in many publications such as Kontermann et al [70] and WO92/01047 (discussed further below). Transgenic mice in which the mouse antibody genes are inactivated and functionally replaced with human antibody genes while leaving intact other components of the mouse immune system, can be used for isolating human antibodies to human antigens [71].

Synthetic antibody molecules can be created by expression from genes generated by means of oligonucleotides synthesized and assembled within suitable expression vectors, for example as described by Knappik et al. J. Mol. Biol. (2000) 296, 57-86 or Krebs et al. Journal of Immunological Methods 254 2001 67-84.

It has been shown that fragments of a whole antibody can perform the function of binding antigens. Examples of binding fragments are (i) the Fab fragment consisting of VL, VH, CL, and CH1 domains; (ii) the Fd fragment consisting of the VH and CH1 domains; (iii) the Fv fragment consisting of the VL and VH domains of a single antibody; (iv) the dAb fragment (Ward, E. S. et al., Nature 341, 544-546 (1989), McCafferty et al (1990) Nature, 348, 552-554) which consists of a VH domain; (v) isolated CDR regions; (vi) F(ab′)2 fragments, a bivalent fragment comprising two linked Fab fragments (vii) single chain Fv molecules (scFv), wherein a VH domain and a VL domain are linked by a peptide linker which allows the two domains to associate to form an antigen-binding site (Bird et al, Science, 242, 423-426, 1988; Huston et al, PNAS USA, 85, 5879-5883, 1988); (viii) bispecific single chain Fv dimers (PCT/US92/09965) and (ix) “diabodies,” multivalent or multispecific fragments constructed by gene fusion (WO94/13804; P. Holliger et al, Proc. Natl. Acad. Sci. USA 90 6444-6448, 1993). Fv, scFv or diabody molecules may be stabilised by the incorporation of disulphide bridges linking the VH and VL domains (Y. Reiter et al, Nature Biotech, 14, 1239-1245, 1996). Minibodies comprising a scFv joined to a CH3 domain may also be made (S. Hu et al, Cancer Res., 56, 3055-3061, 1996).

Where bispecific antibodies are to be used, these may be conventional bispecific antibodies, which can be manufactured in a variety of ways (Holliger, P. and Winter G. Current Opinion Biotechnol. 4, 446-449 (1993)), e.g. prepared chemically or from hybrid hybridomas, or may be any of the bispecific antibody fragments mentioned above. Examples of bispecific antibodies include those of the BiTE™ technology in which the binding domains of two antibodies with different specificity can be used and directly linked via short flexible peptides. This combines two antibodies on a short single polypeptide chain. Diabodies and scFv can be constructed without an Fc region, using only variable domains, potentially reducing the effects of anti-idiotypic reaction.

Bispecific diabodies, as opposed to bispecific whole antibodies, may also be particularly useful because they can be readily constructed and expressed in E. coli. Diabodies (and many other polypeptides such as antibody fragments) of appropriate binding specificities can be readily selected using phage display (WO94/13804) from libraries. If one arm of the diabody is to be kept constant, for instance, with a specificity directed against IL-13, then a library can be made where the other arm is varied and an antibody of appropriate specificity selected. Bispecific whole antibodies may be made by knobs-into-holes engineering (J. B. B. Ridgeway et al, Protein Eng., 9, 616-621, 1996).

The term “specific” may be used to refer to the situation in which one member of a specific binding pair will not show any significant binding to molecules other than its specific binding partner(s). The term is also applicable where e.g. an antigen-binding domain is specific for a particular epitope which is carried by a number of antigens, in which case the antigen-binding protein carrying the antigen-binding domain will be able to bind to the various antigens carrying the epitope.

By “specifically binds” it is generally meant that an antigen-binding protein including an antibody or antigen-binding fragment, variant, or derivative thereof binds to an epitope via its antigen-binding domain, and that the binding entails some complementarity between the antigen-binding domain and the epitope. According to this definition, an antibody is said to “specifically bind” to an epitope when it binds to that epitope via its antigen-binding domain more readily than it would bind to a random, unrelated epitope.

“Affinity” is a measure of the intrinsic binding strength of a ligand binding reaction. For example, a measure of the strength of the antibody (Ab)-antigen (Ag) interaction is measured through the binding affinity, which may be quantified by the dissociation constant, k_{d}. The dissociation constant is the binding affinity constant and is given by:

$$Kd = \frac{[ Ab ] [ Ag ]}{[ AbAg complex ]}$$

Affinity may, for example, be measured using a BIAcore® and/or a KinExA affinity assay.

“Potency” is a measure of pharmacological activity of a compound expressed in terms of the amount of the compound required to produce an effect of given intensity. It refers to the amount of the compound required to achieve a defined biological effect; the smaller the dose required, the more potent the drug. Potency of an antigen-binding protein that binds IL-13 may, for example, be determined using a TF1 proliferation assay, as described herein.

An antigen-binding protein including an antibody or antigen-binding fragment, variant, or derivative thereof is said to competitively inhibit binding of a reference antibody or antigen-binding fragment thereof to a given epitope or “compete” with a reference antibody or antigen-binding fragment if it blocks, to some degree, binding of the reference antibody or antigen-binding fragment to the epitope. Competitive inhibition can be determined by any method known in the art, for example, competition ELISA assays. A binding molecule can be said to competitively inhibit binding of the reference antibody or antigen-binding fragment to a given epitope or compete with a reference antibody or antigen-binding fragment thereof by at least 90%, at least 80%, at least 70%, at least 60%, or at least 50%.

The term “compete” when used in the context of antigen-binding proteins (e.g., neutralizing antigen-binding proteins or neutralizing antibodies) means competition between antigen-binding proteins as determined by an assay in which the antigen-binding protein (e.g., antibody or immunologically functional fragment thereof) under test prevents or inhibits specific binding of a reference antigen-binding protein (e.g., a ligand, or a reference antibody) to a common antigen (e.g., an IL-13 protein or a fragment thereof). Numerous types of competitive binding assays can be used, for example: solid phase direct or indirect radioimmunoassay (RIA), solid phase direct or indirect enzyme immunoassay (EIA), sandwich competition assay (see, e.g., Stahli et al., 1983, Methods in Enzymology 92:242-253); solid phase direct biotin-avidin EIA (see, e.g., Kirkland et al., 1986, J. Immunol. 137:3614-3619) solid phase direct labeled assay, solid phase direct labeled sandwich assay (see, e.g., Harlow and Lane, 1988, Antibodies, A Laboratory Manual, Cold Spring Harbor Press); solid phase direct label RIA using 1-125 label (see, e.g., Morel et al., 1988, Molec. Immunol. 25:7-15); solid phase direct biotin-avidin EIA (see, e.g., Cheung, et al., 1990, Virology 176:546-552); and direct labeled RIA (Moldenhauer et al., 1990, Scand. J. Immunol. 32:77-82). Typically, such an assay involves the use of purified antigen bound to a solid surface or cells bearing either of these, an unlabelled test antigen-binding protein and a labeled reference antigen-binding protein.

Competitive inhibition can be measured by determining the amount of label bound to the solid surface or cells in the presence of the test antigen-binding protein. Usually the test antigen-binding protein is present in excess. Antigen-binding proteins identified by competition assay (competing antigen-binding proteins) include antigen-binding proteins binding to the same epitope as the reference antigen-binding proteins and antigen-binding proteins binding to an adjacent epitope sufficiently proximal to the epitope bound by the reference antigen-binding protein for steric hindrance to occur. Usually, when a competing antigen-binding protein is present in excess, it will inhibit specific binding of a reference antigen-binding protein to a common antigen by at least 40%, 45%, 50%, 55%, 60%, 65%, 70% or 75%. In some instance, binding is inhibited by at least 80%, 85%, 90%, 91%, 92%, 93%, 94%, 95%, 96%, 97% 98%, 99% or more.

Antigen-binding proteins, antibodies or antigen-binding fragments, variants, or derivatives thereof disclosed herein can be described or specified in terms of the epitope(s) or portion(s) of an antigen, e.g., a target polypeptide that they recognize or specifically bind. For example, the portion of IL-13 that specifically interacts with the antigen-binding domain of the antigen-binding polypeptide or fragment thereof disclosed herein is an “epitope”. Epitopes can be formed both from contiguous amino acids or noncontiguous amino acids juxtaposed by tertiary folding of a protein. Epitopes formed from contiguous amino acids are typically retained on exposure to denaturing solvents, whereas epitopes formed by tertiary folding are typically lost on treatment with denaturing solvents. Epitope determinants may include chemically active surface groupings of molecules such as amino acids, sugar side chains, phosphoryl or sulfonyl groups, and may have specific three dimensional structural characteristics, and/or specific charge characteristics. An epitope typically includes at least 3, 4, 5, 6, 3, 4, 5, 6, 7, 8, 9, 10, 11, 12, 13, 14, 15, 16, 17, 18, 19, 20, 25, 30, 35 amino acids in a unique spatial conformation. Epitopes can be determined using methods known in the art.

Amino acids are referred to herein by either their commonly known three letter symbols or by the one-letter symbols recommended by the IUPAC-IUB Biochemical Nomenclature Commission. Nucleotides, likewise, are referred to by their commonly accepted single-letter codes.

As used herein, the term “polypeptide” refers to a molecule composed of monomers (amino acids) linearly linked by amide bonds (also known as peptide bonds). The term “polypeptide” refers to any chain or chains of two or more amino acids, and does not refer to a specific length of the product. As used herein the term “protein” is intended to encompass a molecule comprised of one or more polypeptides, which can in some instances be associated by bonds other than amide bonds. On the other hand, a protein can also be a single polypeptide chain. In this latter instance the single polypeptide chain can in some instances comprise two or more polypeptide subunits fused together to form a protein. The terms “polypeptide” and “protein” also refer to the products of post-expression modifications, including without limitation glycosylation, acetylation, phosphorylation, amidation, derivatization by known protecting/blocking groups, proteolytic cleavage, or modification by non-naturally occurring amino acids. A polypeptide or protein can be derived from a natural biological source or produced by recombinant technology, but is not necessarily translated from a designated nucleic acid sequence. It can be generated in any manner, including by chemical synthesis.

The term “isolated” refers to the state in which antigen-binding proteins of the disclosure, or nucleic acid encoding such binding proteins, will generally be in accordance with the present disclosure. Isolated proteins and isolated nucleic acid will be free or substantially free of material with which they are naturally associated such as other polypeptides or nucleic acids with which they are found in their natural environment, or the environment in which they are prepared (e.g. cell culture) when such preparation is by recombinant DNA technology practised in vitro or in vivo. Proteins and nucleic acid may be formulated with diluents or adjuvants and still for practical purposes be isolated—for example the proteins will normally be mixed with gelatin or other carriers if used to coat microtitre plates for use in immunoassays, or will be mixed with pharmaceutically acceptable carriers or diluents when used in diagnosis or therapy. Antigen-binding proteins may be glycosylated, either naturally or by systems of heterologous eukaryotic cells (e.g. CHO or NS0 (ECACC 85110503) cells, or they may be (for example if produced by expression in a prokaryotic cell) unglycosylated.

A polypeptide, antigen-binding protein, antibody, polynucleotide, vector, cell, or composition which is “isolated” is a polypeptide, antigen-binding protein, antibody, polynucleotide, vector, cell, or composition which is in a form not found in nature. Isolated polypeptides, antigen-binding proteins, antibodies, polynucleotides, vectors, cells, or compositions include those which have been purified to a degree that they are no longer in a form in which they are found in nature. In some aspects, an antigen-binding protein, antibody, polynucleotide, vector, cell, or composition which is isolated is substantially pure.

A “recombinant” polypeptide, protein or antibody refers to a polypeptide or protein or antibody produced via recombinant DNA technology. Recombinantly produced polypeptides, proteins and antibodies expressed in host cells are considered isolated for the purpose of the present disclosure, as are native or recombinant polypeptides which have been separated, fractionated, or partially or substantially purified by any suitable technique.

Also included in the present disclosure are fragments, variants, or derivatives of polypeptides, and any combination thereof. The term “fragment” when referring to polypeptides and proteins of the present disclosure include any polypeptides or proteins which retain at least some of the properties of the reference polypeptide or protein. Fragments of polypeptides include proteolytic fragments, as well as deletion fragments.

The term “variant” as used herein refers to an antibody or polypeptide sequence that differs from that of a parent antibody or polypeptide sequence by virtue of at least one amino acid modification. Variants of antibodies or polypeptides of the present disclosure include fragments, and also antibodies or polypeptides with altered amino acid sequences due to amino acid substitutions, deletions, or insertions. Variants can be naturally or non-naturally occurring. Non-naturally occurring variants can be produced using art-known mutagenesis techniques. Variant polypeptides can comprise conservative or non-conservative amino acid substitutions, deletions or additions.

The term “derivatives” as applied to antibodies or polypeptides refers to antibodies or polypeptides which have been altered so as to exhibit additional features not found on the native polypeptide or protein. An example of a “derivative” antibody is a fusion or a conjugate with a second polypeptide or another molecule (e.g., a polymer such as PEG, a chromophore, or a fluorophore) or atom (e.g., a radioisotope).

The terms “polynucleotide” or “nucleotide” as used herein are intended to encompass a singular nucleic acid as well as plural nucleic acids, and refers to an isolated nucleic acid molecule or construct, e.g., messenger RNA (mRNA) or plasmid DNA (pDNA). In certain aspects, a polynucleotide comprises a conventional phosphodiester bond or a non-conventional bond (e.g., an amide bond, such as found in peptide nucleic acids (PNA)).

The term “nucleic acid” refers to any one or more nucleic acid segments, e.g., DNA or RNA fragments, present in a polynucleotide. When applied to a nucleic acid or polynucleotide, the term “isolated” refers to a nucleic acid molecule, DNA or RNA, which has been removed from its native environment, for example, a recombinant polynucleotide encoding an antigen-binding protein contained in a vector is considered isolated for the purposes of the present disclosure. Further examples of an isolated polynucleotide include recombinant polynucleotides maintained in heterologous host cells or purified (partially or substantially) from other polynucleotides in a solution. Isolated RNA molecules include in vivo or in vitro RNA transcripts of polynucleotides of the present disclosure. Isolated polynucleotides or nucleic acids according to the present disclosure further include such molecules produced synthetically. In addition, a polynucleotide or a nucleic acid can include regulatory elements such as promoters, enhancers, ribosome binding sites, or transcription termination signals.

As used herein, the term “host cell” refers to a cell or a population of cells harboring or capable of harboring a recombinant nucleic acid. Host cells can be a prokaryotic cells (e.g., E. coli), or alternatively, the host cells can be eukaryotic, for example, fungal cells (e.g., yeast cells such as Saccharomyces cerivisiae, Pichia pastoris, or Schizosaccharomyces pombe), and various animal cells, such as insect cells (e.g., Sf-9) or mammalian cells (e.g., HEK293F, CHO, COS-7, NIH-3T3, a NS0 murine myeloma cell, a PER.C6® human cell, a Chinese hamster ovary (CHO) cell or a hybridoma).

The term “percent sequence identity” or “percent identity” between two polynucleotide or polypeptide sequences refers to the number of identical matched positions shared by the sequences over a comparison window, taking into account additions or deletions (i.e., gaps) that must be introduced for optimal alignment of the two sequences. A matched position is any position where an identical nucleotide or amino acid is presented in both the target and reference sequence. Gaps presented in the target sequence are not counted since gaps are not nucleotides or amino acids. Likewise, gaps presented in the reference sequence are not counted since target sequence nucleotides or amino acids are counted, not nucleotides or amino acids from the reference sequence. The percentage of sequence identity is calculated by determining the number of positions at which the identical amino-acid residue or nucleic acid base occurs in both sequences to yield the number of matched positions, dividing the number of matched positions by the total number of positions in the window of comparison and multiplying the result by 100 to yield the percentage of sequence identity. The comparison of sequences and determination of percent sequence identity between two sequences can be accomplished using readily available software programs. Suitable software programs are available from various sources, and for alignment of both protein and nucleotide sequences. One suitable program to determine percent sequence identity is bl2seq, part of the BLAST suite of program available from the U.S. government's National Center for Biotechnology Information BLAST web site (blast.ncbi.nlm.nih.gov). Bl2seq performs a comparison between two sequences using either the BLASTN or BLASTP algorithm. BLASTN is used to compare nucleic acid sequences, while BLASTP is used to compare amino acid sequences. Other suitable programs are, e.g., Needle, Stretcher, Water, or Matcher, part of the EMBOSS suite of bioinformatics programs and also available from the European Bioinformatics Institute (EBI) at www.ebi.ac.uk/Tools/psa.

“Specific binding member” describes a member of a pair of molecules which have binding specificity for one another. The members of a specific binding pair may be naturally derived or wholly or partially synthetically produced. One member of the pair of molecules has an area on its surface, or a cavity, which specifically binds to and is therefore complementary to a particular spatial and polar organisation of the other member of the pair of molecules. Thus the members of the pair have the property of binding specifically to each other. Examples of types of specific binding pairs are antigen-antibody, biotin-avidin, hormone-hormone receptor, receptor-ligand, enzyme-substrate. The present disclosure is concerned with antigen-antibody type reactions.

The term “IgG” as used herein refers to a polypeptide belonging to the class of antibodies that are substantially encoded by a recognized immunoglobulin gamma gene. In humans this class comprises IgG1, IgG2, IgG3, and IgG4. In mice this class comprises IgG1, IgG2a, IgG2b, and IgG3.

The term “antigen-binding domain” describes the part of an antibody molecule which comprises the area which specifically binds to and is complementary to part or all of an antigen. Where an antigen is large, an antibody may only bind to a particular part of the antigen, which part is termed an epitope. An antigen-binding domain may be provided by one or more antibody variable domains (e.g. a so-called Fd antibody fragment consisting of a VH domain). An antigen-binding domain may comprise an antibody light chain variable region (VL) and an antibody heavy chain variable region (VH).

The term “antigen-binding protein fragment” or “antibody fragment” refers to a portion of an intact antigen-binding protein or antibody and refers to the antigenic determining variable regions of an intact antigen-binding protein or antibody. It is known in the art that the antigen-binding function of an antibody can be performed by fragments of a full-length antibody. Examples of antibody fragments include, but are not limited to Fab, Fab′, F(ab′)2, and Fv fragments, linear antibodies, single chain antibodies, and multispecific antibodies formed from antibody fragments.

The term “monoclonal antibody” refers to a homogeneous antibody population involved in the highly specific recognition and binding of a single antigenic determinant, or epitope. This is in contrast to polyclonal antibodies that typically include different antibodies directed against different antigenic determinants. The term “monoclonal antibody” encompasses both intact and full-length monoclonal antibodies as well as antibody fragments (such as Fab, Fab′, F(ab′)2, Fv), single chain (scFv) mutants, fusion proteins comprising an antibody portion, and any other modified immunoglobulin molecule comprising an antigen recognition site. Furthermore, “monoclonal antibody” refers to such antibodies made in any number of ways including, but not limited to, by hybridoma, phage selection, recombinant expression, and transgenic animals.

The term “human antibody” refers to an antibody produced by a human or an antibody having an amino acid sequence corresponding to an antibody produced by a human made using any technique known in the art. This definition of a human antibody includes intact or full-length antibodies, fragments thereof, and/or antibodies comprising at least one human heavy and/or light chain polypeptide such as, for example, an antibody comprising murine light chain and human heavy chain polypeptides. The term “humanized antibody” refers to an antibody derived from a non-human (e.g., murine) immunoglobulin, which has been engineered to contain minimal non-human (e.g., murine) sequences.

The term “chimeric antibody” refers to antibodies wherein the amino acid sequence of the immunoglobulin molecule is derived from two or more species. Typically, the variable region of both light and heavy chains corresponds to the variable region of antibodies derived from one species of mammals (e.g., mouse, rat, rabbit, etc) with the desired specificity, affinity, and capability while the constant regions are homologous to the sequences in antibodies derived from another (usually human) to avoid eliciting an immune response in that species.

The term “EU index as in Kabat” refers to the numbering system of the human IgG1 EU antibody described in Kabat et al., Sequences of Immunological Interest, 5th Ed. Public Health Service, National Institutes of Health, Bethesda, Md. (1991). All amino acid positions referenced in the present application refer to EU index positions. For example, both “L234” and “EU L234” refer to the amino acid leucine at position 234 according to the EU index as set forth in Kabat.

The terms “Fc domain,” “Fc Region,” and “IgG Fc domain” as used herein refer to the portion of an immunoglobulin, e.g., an IgG molecule, that correlates to a crystallizable fragment obtained by papain digestion of an IgG molecule. The Fc region comprises the C-terminal half of two heavy chains of an IgG molecule that are linked by disulfide bonds. It has no antigen-binding activity but contains the carbohydrate moiety and binding sites for complement and Fc receptors, including the FcRn receptor. For example, an Fc domain contains the entire second constant domain CH2 (residues at EU positions 231-340 of human IgG1) and the third constant domain CH3 (residues at EU positions 341-447 of human IgG1).

Fc can refer to this region in isolation, or this region in the context of an antibody, antibody fragment, or Fc fusion protein. Polymorphisms have been observed at a number of positions in Fc domains, including but not limited to EU positions 270, 272, 312, 315, 356, and 358. Thus, a “wild type IgG Fc domain” or “WT IgG Fc domain” refers to any naturally occurring IgG Fc region (i.e., any allele). Myriad Fc mutants, Fc fragments, Fc variants, and Fc derivatives are described, e.g., in U.S. Pat. Nos. 5,624,821; 5,885,573; 5,677,425; 6,165,745; 6,277,375; 5,869,046; 6,121,022; 5, 624, 821; 5, 648, 260; 6,528,624; 6,194,551; 6,737,056; 7,122,637; 7,183,387; 7,332,581; 7,335,742; 7,371,826; 6,821,505; 6,180,377; 7,317,091; 7,355,008; U.S. Patent publication 2004/0002587; and PCT Publication Nos. WO 99/058572, WO 2011/069164 and WO 2012/006635.

The sequences of the heavy chains of human IgG1, IgG2, IgG3 and IgG4 can be found in a number of sequence databases, for example, at the Uniprot database (www.uniprot.org) under accession numbers P01857 (IGHG1_HUMAN), P01859 (IGHG2_HUMAN), P01860 (IGHG3_HUMAN), and P01861 (IGHG1_HUMAN), respectively.

The terms “YTE” or “YTE mutant” refer to a set of mutations in an IgG1 Fc domain that results in an increase in the binding to human FcRn and improves the serum half-life of the antibody having the mutation. A YTE mutant comprises a combination of three “YTE mutations”: M252Y, S254T, and T256E, wherein the numbering is according to the EU index as in Kabat, introduced into the heavy chain of an IgG. See U.S. Pat. No. 7,658,921, which is incorporated by reference herein. The YTE mutant has been shown to increase the serum half-life of antibodies compared to wild-type versions of the same antibody. See, e.g., Dall'Acqua et al., J. Biol. Chem. 281:23514-24 (2006) and U.S. Pat. No. 7,083,784, which are hereby incorporated by reference in their entireties. A “Y” mutant comprises only the M256Y mutations; similarly a “YT” mutation comprises only the M252Y and S254T; and a “YE” mutation comprises only the M252Y and T256E. It is specifically contemplated that other mutations may be present at EU positions 252 and/or 256. In certain aspects, the mutation at EU position 252 may be M252F, M252S, M252W or M252T and/or the mutation at EU position 256 may be T256S, T256R, T256Q or T256D.

The term “naturally occurring IL-13” generally refers to a state in which the IL-13 protein or fragments thereof may occur. Naturally occurring IL-13 means IL-13 protein which is naturally produced by a cell, without prior introduction of encoding nucleic acid using recombinant technology. Thus, naturally occurring IL-13 may be as produced naturally by for example CD4+ T cells and/or as isolated from a mammal, e.g. human, non-human primate, rodent such as rat or mouse.

The term “recombinant IL-13” refers to a state in which the IL-13 protein or fragments thereof may occur. Recombinant IL-13 means IL-13 protein or fragments thereof produced by recombinant DNA, e.g., in a heterologous host. Recombinant IL-13 may differ from naturally occurring IL-13 by glycosylation.

Recombinant proteins expressed in prokaryotic bacterial expression systems are not glycosylated while those expressed in eukaryotic systems such as mammalian or insect cells are glycosylated. Proteins expressed in insect cells however differ in glycosylation from proteins expressed in mammalian cells.

The terms “half-life” or “in vivo half-life” as used herein refer to the biological half-life of a particular type of antibody, antigen-binding protein, or polypeptide of the present disclosure in the circulation of a given animal and is represented by a time required for half the quantity administered in the animal to be cleared from the circulation and/or other tissues in the animal.

The term “subject” as used herein refers to any animal (e.g., a mammal), including, but not limited to humans, non-human primates, rodents, sheep, dogs, cats, horses, cows, bears, chickens, amphibians, reptiles, and the like, which is to be the recipient of a particular treatment. The terms “subject” and “patient” as used herein refer to any subject, particularly a mammalian subject, for whom diagnosis, prognosis, or therapy of an IL-13-mediated disease or condition is desired. As used herein, phrases such as “a patient having an IL-13-mediated disease or condition” includes subjects, such as mammalian subjects, that would benefit from the administration of a therapy, imaging or other diagnostic procedure, and/or preventive treatment for that IL-13-mediated disease or condition.

The term “pharmaceutical composition” as used herein refers to a preparation which is in such form as to permit the biological activity of the active ingredient to be effective, and which contains no additional components which are unacceptably toxic to a subject to which the composition would be administered. Such composition can be sterile.

An “effective amount” of a polypeptide, e.g., an antigen-binding protein including an antibody, as disclosed herein is an amount sufficient to carry out a specifically stated purpose. An “effective amount” can be determined empirically and in a routine manner, in relation to the stated purpose. The term “therapeutically effective amount” as used herein refers to an amount of a polypeptide, e.g., an antigen-binding protein including an antibody, or other drug effective to “treat” a disease or condition in a subject or mammal and provides some improvement or benefit to a subject having an IL-13-mediated disease or condition. Thus, a “therapeutically effective” amount is an amount that provides some alleviation, mitigation, and/or decrease in at least one clinical symptom of the IL-13-mediated disease or condition. Clinical symptoms associated with the IL-13-mediated disease or condition that can be treated by the methods and systems of the disclosure are well known to those skilled in the art. Further, those skilled in the art will appreciate that the therapeutic effects need not be complete or curative, as long as some benefit is provided to the subject. In some aspects, the term “therapeutically effective” refers to an amount of a therapeutic agent that is capable of reducing IL-13 activity in a patient in need thereof. The actual amount administered and rate and time-course of administration, will depend on the nature and severity of what is being treated. Prescription of treatment, e.g. decisions on dosage etc, is within the responsibility of general practitioners and other medical doctors. Appropriate doses of antibodies and antigen-binding fragments thereof are well known in the art; see Ledermann J. A. et al. (1991) Int. J. Cancer 47: 659-664; Bagshawe K. D. et al. (1991) Antibody, Immunoconjugates and Radiopharmaceuticals 4: 915-922.

As used herein, a “sufficient amount” or “an amount sufficient to” achieve a particular result in a patient having an IL-13-mediated disease or condition refers to an amount of a therapeutic agent (e.g., an antigen-binding protein including an antibody, as disclosed herein) that is effective to produce a desired effect, which is optionally a therapeutic effect (i.e., by administration of a therapeutically effective amount). In some aspects, such particular result is a reduction in IL-13 activity in a patient in need thereof.

The term “label” when used herein refers to a detectable compound or composition which is conjugated directly or indirectly to a polypeptide, e.g., an antigen-binding protein including an antibody, so as to generate a “labeled” polypeptide or antibody. The label can be detectable by itself (e.g., radioisotope labels or fluorescent labels) or, in the case of an enzymatic label, can catalyze chemical alteration of a substrate compound or composition which is detectable.

Terms such as “treating” or “treatment” or “to treat” or “alleviating” or “to alleviate” or “ameliorating” or “or ameliorate” refer to therapeutic measures that cure, slow down, lessen symptoms of, and/or halt progression of a diagnosed pathologic condition or disorder. Terms such as “preventing” refer to prophylactic or preventative measures that prevent and/or slow the development of a targeted pathologic condition or disorder. Thus, those in need of treatment include those already with the disease or condition. Those in need of prevention include those prone to have the disease or condition and those in whom the disease or condition is to be prevented. For example, the phrase “treating a patient having an IL-13-mediated disease or condition” refers to reducing the severity of the IL-13-mediated disease or condition, preferably, to an extent that the subject no longer suffers discomfort and/or altered function due to it (for example, a relative reduction in asthma exacerbations when compared to untreated patients). The phrase “preventing an IL-13-mediated disease or condition” refers to reducing the potential for an IL-13-mediated disease or condition and/or reducing the occurrence of the IL-13-mediated disease or condition.

The term “vector” means a construct, which is capable of delivering, and in some aspects, expressing, one or more gene(s) or sequence(s) of interest in a host cell. Examples of vectors include, but are not limited to, viral vectors, naked DNA or RNA expression vectors, plasmid, cosmid or phage vectors, DNA or RNA expression vectors associated with cationic condensing agents, DNA or RNA expression vectors encapsulated in liposomes, and certain eukaryotic cells, such as producer cells.

The methods and techniques of the present disclosure are generally performed according to conventional methods well known in the art and as described in various general and more specific references that are cited and discussed throughout the present specification unless otherwise indicated. See, e.g., Sambrook et al., Molecular Cloning: A Laboratory Manual, 3rd ed., Cold Spring Harbor Laboratory Press, Cold Spring Harbor, N.Y. (2001) and Ausubel et al., Current Protocols in Molecular Biology, Greene Publishing Associates (1992), and Harlow and Lane Antibodies: A Laboratory Manual Cold Spring Harbor Laboratory Press, Cold Spring Harbor, N.Y. (1990), all of which are herein incorporated by reference.

As used herein, the term “IL-13-mediated disease or condition” refers to any pathology caused by (alone or in association with other mediators), exacerbated by, associated with, or prolonged by abnormal levels of IL-13 in the subject having the disease or condition. Non-limiting examples of IL-13-mediated diseases or conditions include asthma, idiopathic pulmonary fibrosis (IPF), chronic obstructive pulmonary disease (COPD), ulcerative colitis (UC), atopic dermatitis, allergic rhinitis, chronic rhinosinusitis, fibrosis, scleroderma, systemic sclerosis, pulmonary fibrosis, liver fibrosis, inflammatory bowel disease, Sjögren's Syndrome or Hodgkin's lymphoma.

The term “asthma” refers to diseases that present as reversible airflow obstruction and/or bronchial hyper-responsiveness that may or may not be associated with underlying inflammation. Examples of asthma include allergic asthma, atopic asthma, corticosteroid naive asthma, chronic asthma, corticosteroid resistant asthma, corticosteroid refractory asthma, asthma due to smoking, asthma uncontrolled on corticosteroids and other asthmas as mentioned, e.g., in the Expert Panel Report 3: Guidelines for the Diagnosis and Management of Asthma, National Asthma Education and Prevention Program (2007) (“NAEPP Guidelines”), incorporated herein by reference in its entirety.

The term “COPD” as used herein refers to chronic obstructive pulmonary disease. The term “COPD” includes two main conditions: emphysema and chronic obstructive bronchitis.

The term “Idiopathic Pulmonary Fibrosis” (IPF) refers to a disease characterized by progressive scarring, or fibrosis, of the lungs. It is a specific type of interstitial lung disease in which the alveoli gradually become replaced by fibrotic tissue. With IPF, progressive scarring causes the normally thin and pliable tissue to thicken and become stiff, making it more difficult for the lungs to expand, preventing oxygen from readily getting into the bloodstream. See, e.g., Am. J. Respir. Crit. Care Med. 2000. 161:646-664.

The term “BAK1183H4 antibody,” “BAK1183H4,” “1183H4”, “1183H04” or “BAK1183H4 clone” refers to an anti-IL-13 antibody described in WO 2005/007699 and U.S. Pat. No. 7,829,090, each herein incorporated by reference. The BAK1183H4 antibody comprises a VH domain (SEQ ID NO: 2) and a VL domain (SEQ ID NO: 7) containing a set of CDRs HCDR1, HCDR2, HCDR3, LCDR1, LCDR2 and LCDR3, wherein HCDR1 comprises the amino acid sequence of SEQ ID NO: 3, HCDR2 comprises the amino acid sequence of SEQ ID NO: 4, HCDR3 comprises the amino acid sequence of SEQ ID NO: 5, LCDR1 comprises the amino acid sequence of SEQ ID NO: 8, LCDR2 comprises the amino acid sequence of SEQ ID NO: 9, and LCDR3 comprises the amino acid sequence of SEQ ID NO: 10.

The set of CDRs wherein the HCDR1 has the amino acid sequence of SEQ ID NO: 3, the HCDR2 has the amino acid sequence of SEQ ID NO: 4, the HCDR3 has the amino acid sequence of SEQ ID NO: 5, the LCDR1 has the amino acid sequence of SEQ ID NO: 8, the LCDR2 has the amino acid sequence of SEQ ID NO: 9, and the LCDR3 has the amino acid sequence of SEQ ID NO: 10, are herein referred to as the “BAK1183H4 set of CDRs”. The HCDR1, HCDR2 and HCDR3 within the BAK1183H4 set of CDRs are referred to as the “BAK1183H4 set of HCDRs” and the LCDR1, LCDR2 and LCDR3 within the BAK1183H4 set of CDRs are referred to as the “BAK1183H4 set of LCDRs”. A set of CDRs with the BAK1183H4 set of CDRs, BAK1183H4 set of HCDRs or BAK1183H4 set of LCDRs, or one or two substitutions within each CDR, is said to be of the BAK1183H4 lineage.

By “substantially as set out” it is meant that the relevant CDR or VH or VL domain will be either identical or highly similar to the specified regions of which the sequence is set out herein. By “highly similar” it is contemplated that from 1 to 5, e.g. from 1 to 4 such as 1 to 3 or 1 or 2, or 3 or 4, amino acid substitutions can be included in the CDR and/or VH or VL domain.

The structure for carrying a CDR or a set of CDRs will generally be of an antibody heavy or light chain sequence or substantial portion thereof in which the CDR or set of CDRs is located at a location corresponding to the CDR or set of CDRs of naturally occurring VH and VL antibody variable domains encoded by rearranged immunoglobulin genes. The structures and locations of immunoglobulin variable domains may be determined by reference to Kabat, E. A. et al, Sequences of Proteins of Immunological Interest. 4th Edition. US Department of Health and Human Services. 1987, and updates thereof, now available on the Internet (immuno.bme.nwu.edu or find “Kabat” using any search engine), herein incorporated by reference.

CDRs can also be carried by other scaffolds such as fibronectin or cytochrome B [76, 77].

A CDR amino acid sequence substantially as set out herein can be carried as a CDR in a human variable domain or a substantial portion thereof. The HCDR3 sequences substantially as set out herein represent embodiments of the present disclosure and each of these may be carried as a HCDR3 in a human heavy chain variable domain or a substantial portion thereof.

Variable domains employed in the disclosure can be obtained from any germ-line or rearranged human variable domain, or can be a synthetic variable domain based on consensus sequences of known human variable domains. A CDR sequence (e.g. CDR3) can be introduced into a repertoire of variable domains lacking a CDR (e.g. CDR3), using recombinant DNA technology.

For example, Marks et al. (Bio/Technology, 1992, 10:779-783; which is incorporated herein by reference) provide methods of producing repertoires of antibody variable domains in which consensus primers directed at or adjacent to the 5′ end of the variable domain area are used in conjunction with consensus primers to the third framework region of human VH genes to provide a repertoire of VH variable domains lacking a CDR3. Marks et al. further describe how this repertoire can be combined with a CDR3 of a particular antibody. Using analogous techniques, the CDR3-derived sequences of the present disclosure can be shuffled with repertoires of VH or VL domains lacking a CDR3, and the shuffled complete VH or VL domains combined with a cognate VL or VH domain to provide antigen-binding proteins. The repertoire can then be displayed in a suitable host system such as the phage display system of WO92/01047 or any of a subsequent large body of literature, including Kay, B. K., Winter, J., and McCafferty, J. (1996) Phage Display of Peptides and Proteins: A Laboratory Manual, San Diego: Academic Press, so that suitable antigen-binding proteins may be selected. A repertoire can consist of from anything from 10⁴ individual members upwards, for example from 10⁶ to 10⁸ or 10¹⁰ members. Other suitable host systems include yeast display, bacterial display, T7 display, ribosome display and so on. For a review of ribosome display for see Lowe D and Jermutus L, 2004, Curr. Pharm, Biotech, 517-27, also WO92/01047, which are herein incorporated by reference.

Analogous shuffling or combinatorial techniques are also disclosed by Stemmer (Nature, 1994, 370:389-391, which is herein incorporated by reference), who describes the technique in relation to a β-lactamase gene but observes that the approach may be used for the generation of antibodies.

A further alternative is to generate novel VH or VL regions carrying CDR-derived sequences of the disclosure using random mutagenesis of one or more selected VH and/or VL genes to generate mutations within the entire variable domain. Such a technique is described by Gram et al (1992, Proc. Natl. Acad. Sci., USA, 89:3576-3580), who used error-prone PCR. In some embodiments, one or two amino acid substitutions are made within a set of HCDRs and/or LCDRs.

Another method which may be used is to direct mutagenesis to CDR regions of VH or VL genes. Such techniques are disclosed by Barbas et al, (1994, Proc. Natl. Acad. Sci., USA, 91:3809-3813) and Schier et al (1996, J. Mol. Biol. 263:551-567).

The skilled person will be able to use such techniques described above to provide antigen-binding proteins of the disclosure using routine methodology in the art.

### IL-13 Antigen-Binding Proteins

An “antigen-binding protein” as used herein means a protein that specifically binds a specified target antigen; the antigen as provided herein is IL-13, particularly human IL-13, including native human IL-13. The antigen-binding proteins can impact the ability of IL-13 to interact with its receptor, for example by impacting binding to the receptor. In particular, such antigen-binding proteins totally or partially reduce, inhibit, interfere with or modulate one or more biological activities of IL-13. Such inhibition or neutralization disrupts a biological response in the presence of the antigen-binding protein compared to the response in the absence of the antigen-binding protein and can be determined using assays known in the art and described herein. For example, the IL13-binding proteins provided herein inhibit or reduce TF1 cell proliferation as measured in a TF1 cell proliferation assay (as described, e.g., in Example 2). Reduction of biological activity can be about 20%, 30%, 40%, 50%, 60%, 70%, 80%, 85%, 90%, 91%, 92%, 93%, 94%, 95%, 96%, 97% 98%, 99% or more.

Reference to “an antibody binding protein” herein includes “an antigen-binding fragment thereof” wherever it occurs.

Exemplary isolated antigen-binding proteins of the disclosure include antibodies (e.g. a monoclonal antibody, a recombinant antibody, a human antibody, a humanized antibody, a chimeric antibody, a bi-specific antibody, a multi-specific antibody), or an antibody fragment thereof (e.g. a Fab fragment, a Fab′ fragment, a F(ab′)₂ fragment, a Fv fragment, a diabody, or a single chain antibody molecule (scFv)).

The present disclosure provides antigen-binding proteins or fragments thereof which compete for binding to IL-13 and/or competitively inhibit a BAK1183H4 antibody and which bind to human IL-13 with an affinity better than that of the BAK1183H4 antibody. In some embodiments, the antigen-binding proteins are antibody molecules, whether whole antibody (e.g. IgG, such as IgG1) or antibody fragments (e.g., an antigen-binding portion of an antibody including scFv, Fab, or dAbs), antibody derivatives, or antibody analogs.

An antigen-binding protein can comprise a portion that binds to an antigen and, optionally, a scaffold or framework portion that allows the antigen-binding portion to adopt a conformation that promotes binding of the antigen-binding protein to the antigen. The antigen-binding protein can comprise an alternative protein scaffold or artificial scaffold with grafted CDRs or CDR derivatives.

An antigen-binding site can comprise, consist essentially of, or consist of an antibody VH domain and/or a VL domain. An antigen-binding site may be provided by means of arrangement of CDRs on non-antibody protein scaffolds such as fibronectin or cytochrome B etc. [76, 77]. Scaffolds for engineering novel binding sites in proteins have been reviewed in detail by Nygren et al [77]. Protein scaffolds for antibody mimics are disclosed in WO 00/34784 in proteins (antibody mimics) that include a fibronectin type III domain having at least one randomised loop are provided. A suitable scaffold into which to graft one or more CDRs, e.g. a set of HCDRs, can be provided by any domain member of the immunoglobulin gene superfamily.

Some embodiments of the present disclosure are in what is termed herein the “BAK1183H4 lineage”. This is defined with reference to a set of six CDR sequences of BAK1183H4 as follows: HCDR1 (SEQ ID NO: 3), HCDR2 (SEQ ID NO: 4), HCDR3 (SEQ ID NO: 5), LCDR1 (SEQ ID NO: 8), LCDR2 (SEQ ID NO: 9) and LCDR3 (SEQ ID NO: 10). Antigen-binding proteins of the BAK1183H4 lineage as provided by the disclosure have been generated by light chain randomisation of the BAK1183H4 antibody. They therefore retain the BAK1183H4 variable heavy chain (VH) domain sequence, but have one or more mutations in their variable light chain (VL) domain sequence.

In one aspect, the disclosure provides an isolated antigen-binding protein or fragment thereof that binds human IL-13, wherein said antigen-binding protein comprises an antigen-binding site which is composed of a variable heavy (VH) domain and a variable light (VL) domain and which antibody antigen-binding site comprises a set of complementarity determining regions (CDRs), HCDR1, HCDR2, HCDR3, LCDR1, LCDR2 and LCDR3, wherein the VH domain comprises HCDR1, HCDR2 and HCDR3 and the VL domain comprises LCDR1, LCDR2 and LCDR3, and wherein:

HCDR1 comprises the amino acid sequence of SEQ ID NO: 13;
HCDR2 comprises the amino acid sequence of SEQ ID NO: 14;
HCDR3 comprises the amino acid sequence of SEQ ID NO: 15;
LCDR1 comprises the amino acid sequence having the formula:

GGNLX1LX2LX3LX4LX5LVH

wherein LX1 is selected from the group consisting of L and M,
LX2 is selected from the group consisting of L, I and V,
LX3 is selected from the group consisting of G and A,
LX4 is selected from the group consisting of S and A, and
LX5 is selected from the group consisting of R and Y (SEQ ID NO:251);
LCDR2 comprises the amino acid sequence having the formula:

DDLX6DRPS

wherein LX6 is selected from the group consisting of G, I, E, M and Q (SEQ ID NO:252); and
LCDR3 comprises the amino acid sequence having the formula:

QVWDTGSLX7PVV

wherein LX7 is selected from the group consisting of D, R, L and S (SEQ ID NO:253).

In some embodiments, LX1 is selected from the group consisting of L or M,

LX2 is selected from the group consisting of L, I and V,

### LX3 is G,

### LX4 is A,

LX5 is selected from the group consisting of R and Y,
LX6 is selected from the group consisting of G, I, E, M and Q, and
LX7 is selected from the group consisting of D, R, L and S.

In some embodiments, LX1 is selected from the group consisting of L or M, LX2 is selected from the group consisting of L, I and V, LX3 is G, LX4 is A, LX5 is R, LX6 is selected from the group consisting of G, I, E and Q, and LX7 is selected from the group consisting of D, R, L and S.

In some embodiments, LX1 is selected from the group consisting of L or M, LX2 is selected from the group consisting of I or V, LX3 is G, LX4 is A, LX5 is R, LX6 is selected from the group consisting of I, Q and E, and LX7 is selected from the group consisting of R, L and S.

In some embodiments, (i) LX1 is M, LX2 is V, LX3 is G, LX4 is A, LX5 is R, LX6 is E, and LX7 is S; (ii) LX1 is L, LX2 is I, LX3 is G, LX4 is A, LX5 is R, LX6 is I, and LX7 is R; or (iii) LX1 is L, LX2 is I, LX3 is G, LX4 is A, LX5 is R, LX6 is Q, and LX7 is L.

In some embodiments, the antigen-binding protein of the disclosure has a set of 6 CDRs shown for individual clones in Table 3.

In some embodiments, the antigen-binding protein of the disclosure has a set of 6 CDRs shown for individual clones in Table 4.

In some embodiments, the antigen-binding protein of the disclosure has a set of 6 CDRs shown for individual clones in Table 5.

In some embodiments, the antigen-binding protein of the disclosure has a set of 6 CDRs shown for individual clones in Table 6.

In one embodiment, the antigen-binding protein of the disclosure has the HCDR1 sequence shown as SEQ ID NO:13, the HCDR2 sequence shown as SEQ ID NO:14, the HCDR3 sequence shown as SEQ ID NO:15, the LCDR1 sequence shown as SEQ ID NO:18, the LCDR2 sequence shown as SEQ ID NO:19 and the LCDR3 sequence shown as SEQ ID NO:20.

In one embodiment, the antigen-binding protein of the disclosure has the HCDR1 sequence shown as SEQ ID NO:233, the HCDR2 sequence shown as SEQ ID NO:234, the HCDR3 sequence shown as SEQ ID NO:235, the LCDR1 sequence shown as SEQ ID NO:238, the LCDR2 sequence shown as SEQ ID NO:239 and the LCDR3 sequence shown as SEQ ID NO:240 (i.e. clone 13NG0027).

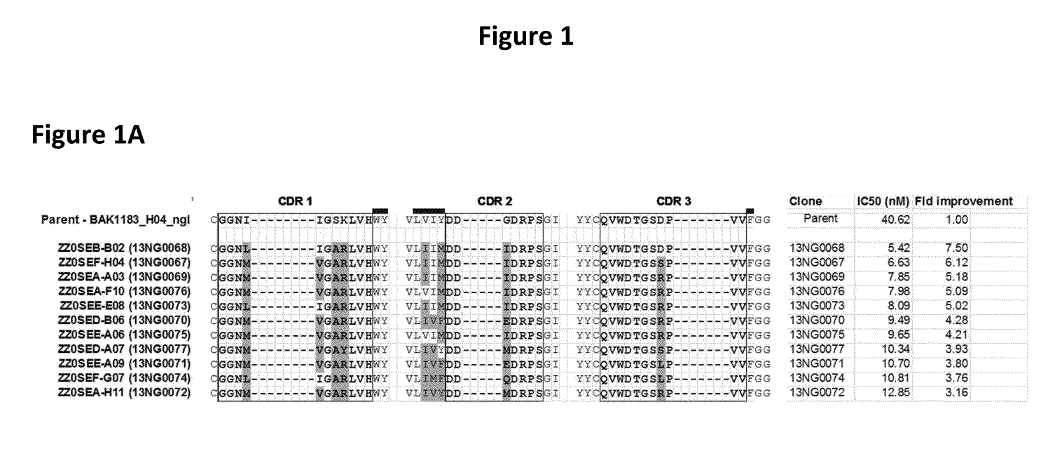

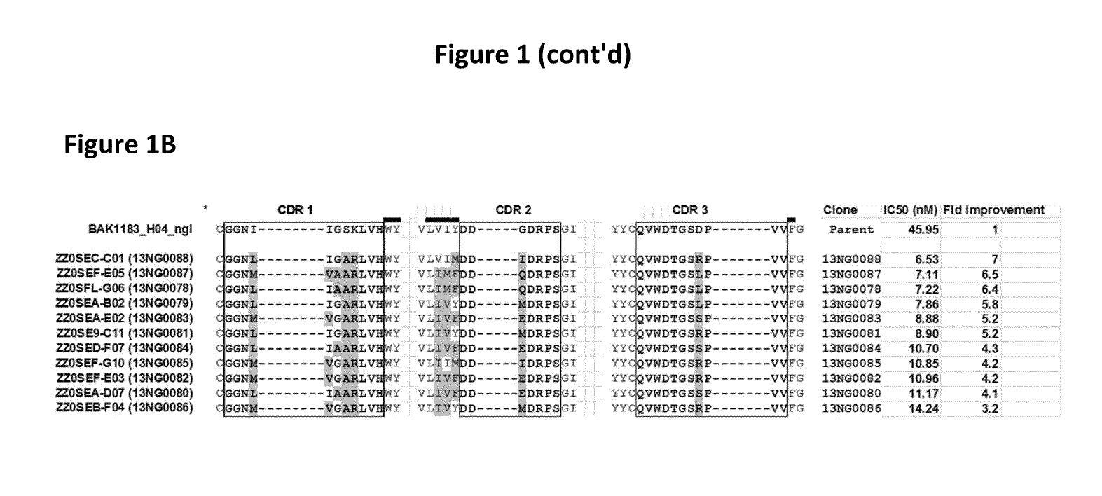

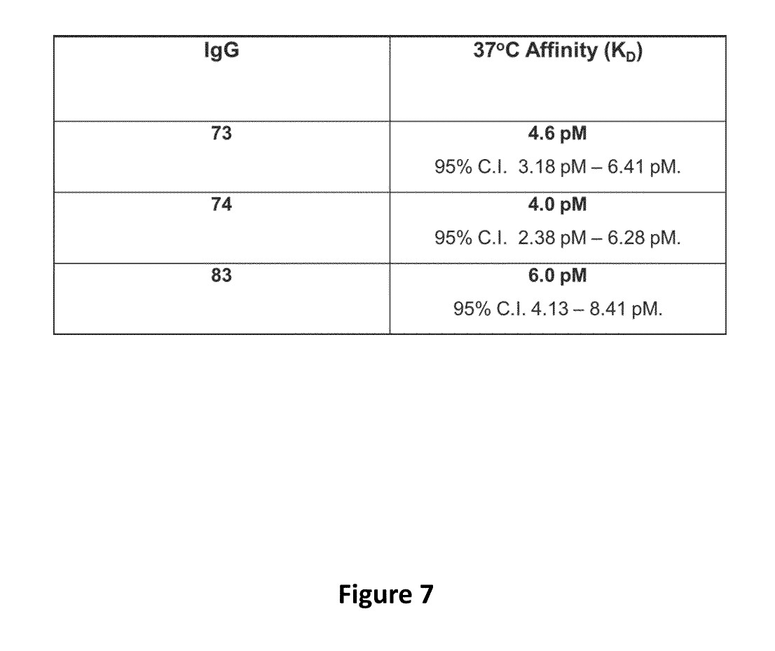

The present inventors have identified the BAK1183H4 lineage as providing human antibody antigen-binding domains against IL-13 with significant improvements in affinity (see FIGS. 1 and 7). Within the lineage, the 13NG0083, 13NG0073, and 13NG0074 clones have been identified as having significant improvements in affinity over the BAK1183H4 parental antibody (see, e.g., FIGS. 1 and 7). The 13NG0083, 13NG0073, and 13NG0074 sets of CDRs are set out in Tables 3-6 below.

The present disclosure also encompasses antigen-binding proteins or polypeptides comprising one or more conservative amino acid substitutions. A “conservative amino acid substitution” is one in which the amino acid residue is replaced with an amino acid residue having a similar side chain. Families of amino acid residues having similar side chains have been defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, if an amino acid in a polypeptide is replaced with another amino acid from the same side chain family, the substitution is considered to be conservative. In another aspect, a string of amino acids can be conservatively replaced with a structurally similar string that differs in order and/or composition of side chain family members.

The relevant set of CDRs is provided within antibody framework regions or other protein scaffold, e.g. fibronectin or cytochrome B [76, 77]. Exemplary antibody framework regions include: germline framework regions, such as DP14 for the antibody framework region of the heavy chain and λ3-3H for the antibody framework region of the light chain and/or any suitable framework regions well known to one of skilled in the art.

The isolated antigen-binding protein of the disclosure may comprise a heavy chain variable region (VH) having at least 90, 95, 97, 98 or 99% sequence identity to SEQ ID NO: 12, 22 or 32 and a light chain variable region (VL) having at least 90, 95, 97, 98 or 99% sequence identity to SEQ ID NO: 17, 27 or 37.

The isolated antigen-binding protein of the disclosure may comprise a VH domain and a VL domain selected from the group consisting of:

(a) a VH domain comprising SEQ ID NO: 12 and a VL domain comprising SEQ ID NO: 17 (13NG0083);
(b) a VH domain comprising SEQ ID NO: 22 and a VL domain comprising SEQ ID NO: 27 (13NG0073);
(c) a VH domain comprising SEQ ID NO: 32 and a VL domain comprising SEQ ID NO: 37 (13NG0074);
(d) a VH domain comprising SEQ ID NO: 112 and a VL domain comprising SEQ ID NO: 117 (13NG0071);
(e) a VH domain comprising SEQ ID NO: 42 and a VL domain comprising SEQ ID NO: 47 (13NG0068);
(f) a VH domain comprising SEQ ID NO: 52 and a VL domain comprising SEQ ID NO: 57 (13NG0067);
(g) a VH domain comprising SEQ ID NO: 62 and a VL domain comprising SEQ ID NO: 67 (13NG0069);
(h) a VH domain comprising SEQ ID NO: 72 and a VL domain comprising SEQ ID NO: 77 (13NG0076);
(i) a VH domain comprising SEQ ID NO: 82 and a VL domain comprising SEQ ID NO: 87 (13NG0070);
(j) a VH domain comprising SEQ ID NO: 92 and a VL domain comprising SEQ ID NO: 97 (13NG0075);
(k) a VH domain comprising SEQ ID NO: 102 and a VL domain comprising SEQ ID NO: 107 (13NG0077); and
(1) a VH domain comprising SEQ ID NO: 122 and a VL domain comprising SEQ ID NO: 127 (13NG0072);
(m) a VH domain comprising SEQ ID NO: 242 and a VL domain comprising SEQ ID NO: 247 (13NG0025);
(n) a VH domain comprising SEQ ID NO: 222 and a VL domain comprising SEQ ID NO: 227 (13NG0078);
(o) a VH domain comprising SEQ ID NO: 142 and a VL domain comprising SEQ ID NO: 147 (13NG0079);
(p) a VH domain comprising SEQ ID NO: 152 and a VL domain comprising SEQ ID NO: 157 (13NG0080);
(q) a VH domain comprising SEQ ID NO: 131 and a VL domain comprising SEQ ID NO: 137 (13NG0081);
(r) a VH domain comprising SEQ ID NO: 192 and a VL domain comprising SEQ ID NO: 197 (13NG0082);
(s) a VH domain comprising SEQ ID NO: 182 and a VL domain comprising SEQ ID NO: 187 (13NG0084);
(t) a VH domain comprising SEQ ID NO: 212 and a VL domain comprising SEQ ID NO: 217 (13NG0085);
(u) a VH domain comprising SEQ ID NO: 162 and a VL domain comprising SEQ ID NO: 167 (13NG0086);
(v) a VH domain comprising SEQ ID NO: 202 and a VL domain comprising SEQ ID NO: 207 (13NG0087); and
(w) a VH domain comprising SEQ ID NO: 172 and a VL domain comprising SEQ ID NO: 177 (13NG0088).

In one embodiment, the antigen-binding protein has a VH domain and a VL domain of a clone selected from:

### 13NG0083 (VH SEQ ID NO: 12, VL SEQ ID NO: 17),

### 13NG0073 (VH SEQ ID NO: 22, VL SEQ ID NO: 27), and

### 13NG0074 (VH SEQ ID NO: 32, VL SEQ ID NO: 37).

In a further embodiment, the present disclosure provides an IgG1 antibody molecule comprising the 13NG0083 VH domain, SEQ ID NO: 12, and the 13NG0083 VL domain, SEQ ID NO: 17. This is termed herein “13NG0083 IgG1”.

In one embodiment, the antigen-binding protein has a VH domain comprising SEQ ID NO:232 and a VL domain comprising SEQ ID NO:237 (clone 13NG0027).

The disclosure also provides other IgG1 antibody molecules, e.g. comprising the 13NG0083 set of HCDRs (SEQ ID NOs: 13-15) within an antibody VH domain, and/or the 13NG0083 set of LCDRs (SEQ ID NOs: 18-20) within an antibody VL domain.

In some embodiments, the antigen-binding protein of the disclosure comprises a set of CDRs, HCDR1, HCDR2, HCDR3, LCDR1, LCDR2 and LCDR3, wherein the set of CDRs is selected from the group consisting of:

(a) HCDR1 comprises the amino acid sequence shown as SEQ ID NO: 13, HCDR2 comprises the amino acid sequence as SEQ ID NO: 14, HCDR3 comprises the amino acid sequence as SEQ ID NO: 15, LCDR1 comprises the amino acid sequence shown as SEQ ID NO: 18, LCDR2 comprises the amino acid sequence shown as SEQ ID NO: 19, and LCDR3 comprises the amino acid sequence shown as SEQ ID NO: 20;
(b) HCDR1 comprises the amino acid sequence shown as SEQ ID NO: 23, HCDR2 comprises the amino acid sequence as SEQ ID NO: 24, HCDR3 comprises the amino acid sequence as SEQ ID NO: 25, LCDR1 comprises the amino acid sequence shown as SEQ ID NO: 28, LCDR2 comprises the amino acid sequence shown as SEQ ID NO: 29, and LCDR3 comprises the amino acid sequence shown as SEQ ID NO: 30; and
(c) HCDR1 comprises the amino acid sequence shown as SEQ ID NO: 33, HCDR2 comprises the amino acid sequence shown as SEQ ID NO: 34, HCDR3 comprises the amino acid sequence shown as SEQ ID NO: 35, LCDR1 comprises the amino acid sequence shown as SEQ ID NO: 38, LCDR2 comprises the amino acid sequence shown as SEQ ID NO: 39, and LCDR3 comprises the amino acid sequence shown as SEQ ID NO: 40.

In some embodiments, the antigen-binding protein of the disclosure comprises a VH domain and a VL domain selected from the group consisting of:

(a) a VH domain comprising SEQ ID NO: 12 and a VL domain comprising SEQ ID NO: 17 (13NG0083);
(b) a VH domain comprising SEQ ID NO: 22 and a VL domain comprising SEQ ID NO: 27 (13NG0073); and
(c) a VH domain comprising SEQ ID NO: 32 and a VL domain comprising SEQ ID NO: 37 (13NG0074).

As noted, the present disclosure provides an antigen-binding protein or fragment thereof which binds human IL-13 and which comprises the 13NG0083 VH domain (SEQ ID NO: 12) and/or the 13NG0083 VL domain (SEQ ID NO: 17).

Generally, a VH domain is paired with a VL domain to provide an antibody antigen-binding site, although as discussed further below a VH domain alone can be used to bind antigen. In one embodiment, the 13NG0083 VH domain (SEQ ID NO: 12) is paired with the 13NG0083 VL domain (SEQ ID NO: 17), so that an antibody antigen-binding site is formed comprising both the 13NG0083 VH and VL domains.

Similarly, any set of HCDRs of the BAK1183H4 lineage can be provided in a VH domain that is used as an antigen-binding protein alone or in combination with a VL domain. A VH domain can be provided with a set of HCDRs of a BAK1183H4 lineage antibody, e.g. as shown in Table 3, and if such a VH domain is paired with a VL domain, then the VL domain may be provided with a set of LCDRs of a BAK1183H4 lineage antibody, e.g. as shown in Table 3. A pairing of a set of HCDRs and a set of LCDRs may be as shown in Table 3, providing an antibody antigen-binding site comprising a set of CDRs as shown in Table 3. The framework regions of the VH and/or VL domains may be germline frameworks. Frameworks regions of the heavy chain domain may be selected from the VH-1 family, and a VH-1 framework is DP-14 framework. Framework regions of the light chain may be selected from the λ3 family, and such a framework is λ3 3H.

One or more CDRs can be taken from the 13NG0083 VH or VL domain and incorporated into a suitable framework. This is discussed further herein. 13NG0083 HCDRs 1, 2 and 3 are shown in SEQ ID NOs: 13-15, respectively. BAK502G9 LCDRs 1, 2 and 3 are shown in SEQ ID NOs: 18-20, respectively.

The same applies for other BAK1183H4 lineage CDRs and sets of CDRs as shown in Tables 3-6.

In the antigen-binding protein of the present disclosure, or antigen-binding fragment thereof, the HCDR1, HCDR2 and HCDR3 can, for example, be within a germ-line framework comprising a set of framework regions HFW1, HFW2, HFW3 and HFW4, wherein:

HFW1 comprises an amino acid sequence having the formula:

QFX1QLVQSGAEVKKPGASVKVSCKASGYTFT,

wherein FX1 is selected from V or A (SEQ ID NO:254);
HFW2 comprises an amino acid sequence having the formula:

WVRQAPGQGLEWFX2G,

wherein FX2 is selected from M and V (SEQ ID NO:255);
HFW3 comprises an amino acid sequence having the formula:

RVTMTTDTSTFX3TAYMELRFX4LRSDDTAVYYCAR,

wherein FX3 is selected from S and G and FX4 is selected from S and G (SEQ ID NO:256); and
HFW4 comprises an amino acid sequence having the formula:

| | |
|---|---|
| W G R G T L V T V S S. | (SEQ ID NO: 257) |

In the antigen-binding protein of the disclosure, or fragment thereof, the LCDR1, LCDR2 and LCDR3 may, for example, be within a germ-line framework comprising a set of framework regions LFW1, LFW2, LFW3 and LFW4, wherein:

LFW1 comprises an amino acid sequence having the formula:

SYVLTQPPFX5VSVAPGKTARIPC,

wherein FX5 is selected from S and L (SEQ ID NO:258);
LFW2 comprises an amino acid sequence having the formula:

WYQQKPGQAPVLFX6FX7FX8,

wherein FX6 is selected from I and V, FX7 is selected from I, M and V, and FX8 is selected from F, Y and M (SEQ ID NO:259);
LFW3 comprises an amino acid sequence having the formula:

GIPERFSGSNSGNTATLTISRVEFX9GDEADYYC,

wherein FX9 is selected from A or T (SEQ ID NO:260); and
LFW4 comprises an amino acid sequence having the formula:

| | |
|---|---|
| F G G G T K L T V L. | (SEQ ID NO: 261) |

In some embodiments, HFW1 comprises an amino acid sequence having the formula:

(SEQ ID NO: 262)
Q V Q L V Q S G A E V K K P G A S V K V S C
K A S G Y T F T;

HFW2 comprises an amino acid sequence having the formula:

| | |
|---|---|
| W V R Q A P G Q G L E W M G; | (SEQ ID NO: 263) |

HFW3 comprises an amino acid sequence having the formula:

(SEQ ID NO: 264)
R V T M T T D T S T S T A Y M E L R S L R S
D D T A V Y Y C A R;

HFW4 comprises an amino acid sequence having the formula:

| | |
|---|---|
| W G R G T L V T V S S; | (SEQ ID NO: 257) |

LFW1 comprises an amino acid sequence having the formula:

(SEQ ID NO: 265)
S Y V L T Q P P S V S V A P G K T A R I P C;

LFW2 comprises an amino acid sequence having the formula:

| | |
|---|---|
| W Y Q Q K P G Q A P V L I V F, | (SEQ ID NO: 266) |
| W Y Q Q K P G Q A P V L I I M, | (SEQ ID NO: 267) |
| W Y Q Q K P G Q A P V L I M F, | (SEQ ID NO: 268) |
| W Y Q Q K P G Q A P V L V I M, | (SEQ ID NO: 269) |
| W Y Q Q K P G Q A P V L I V Y, | (SEQ ID NO: 270) |
| or | |
| W Y Q Q K P G Q A P V L V I Y, | (SEQ ID NO: 271) |

LFW3 comprises an amino acid sequence having the formula:

(SEQ ID NO: 272)
G I P E R F S G S N S G N T A T L T I S R V E
A G D E A D Y Y C;

and
LFW4 comprises an amino acid sequence having the formula:

| | |
|---|---|
| F G G G T K L T V L. | (SEQ ID NO: 261) |

In some embodiments, LFW2 comprises an amino acid sequence having the formula:

(SEQ ID NO: 266; clone 13NG0083),
W Y Q Q K P G Q A P V L I V F
(SEQ ID NO: 267; clone 13NG0073),
W Y Q Q K P G Q A P V L I I M
or
(SEQ ID NO: 268; clone 13NG0074).
W Y Q Q K P G Q A P V L I M F

Variants of the VH and VL domains and CDRs of the present disclosure, including those for which amino acid sequences are set out herein, and which can be employed in antigen-binding proteins for IL-13 can be obtained by means of methods of sequence alteration or mutation and screening.

Variable domain amino acid sequence variants of any of the VH and VL domains whose sequences are specifically disclosed herein can be employed as discussed herein. Particular variants can include one or more amino acid sequence alterations (addition, deletion, substitution and/or insertion of an amino acid residue), can be less than about 20 alterations, less than about 15 alterations, less than about 10 alterations or less than about 5 alterations, 4, 3, 2 or 1. Alterations may be made in one or more framework regions and/or one or more CDRs.

To obtain one or more antigen-binding proteins able to bind the antigen, a library of antigen-binding proteins can be brought into contact with said antigen, and one or more antigen-binding proteins of the library able to bind said antigen selected.

The library can be displayed on the surface of bacteriophage particles, each particle containing nucleic acid encoding the antibody VH variable domain displayed on its surface, and optionally also a displayed VL domain if present.

Following selection of antigen-binding proteins able to bind the antigen and displayed on bacteriophage particles, nucleic acid can be taken from a bacteriophage particle displaying a said selected antigen-binding protein. Such nucleic acid can be used in subsequent production of an antigen-binding protein or an antibody VH variable domain (optionally an antibody VL variable domain) by expression from nucleic acid with the sequence of nucleic acid taken from a bacteriophage particle displaying a said selected antigen-binding protein.

An antibody VH variable domain with the amino acid sequence of an antibody VH variable domain of a said selected antigen-binding protein may be provided in isolated form, as may an antigen-binding protein comprising such a VH domain.

Ability to bind IL-13 may be further tested, also ability to compete with BAK1183H4 (e.g. in scFv format and/or IgG format, e.g. IgG1 or IgG4) for binding to IL-13 or competitively inhibit binding of BAK1183H4 (e.g. in scFv format and/or IgG format, e.g. IgG1 or IgG4) to IL-13. Ability to neutralise IL-13 may be tested, as discussed further below.

The isolated antigen-binding protein provided herein can have one or more properties selected from the group consisting of:
(a) Competes with a BAK1183H4 antibody for binding to IL-13, wherein the BAK1183H4 antibody comprises a VH domain comprising the amino acid sequence of SEQ ID NO: 2 and a VL domain comprising the amino acid sequence of SEQ ID NO: 7;
(b) Binds human IL-13 with an affinity better than that of the BAK1183H4 antibody, wherein the BAK1183H4 antibody comprises a VH domain comprising the amino acid sequence of SEQ ID NO: 2 and a VL domain comprising the amino acid sequence of SEQ ID NO: 7; and
(c) Binds human IL-13 with a KD value of less than about 80 pM, less than about 50 pM, less than about 20 pM, or less than about 10 pM.

An antigen-binding protein according to the present disclosure binds to human IL-13 with an affinity better than that of the BAK1183H4 antibody, the affinity of the antigen-binding protein and the BAK1183H4 antibody being determined under the same conditions. In some embodiments, the antigen-binding protein of the disclosure binds to human IL-3 with a KD value of less than 50 pM, less than 40 pM, less than 30 pM, less than 20 pM, or less than 10 pM.

An antigen-binding protein according to the present disclosure may neutralise human IL-13 with a potency better than that of a BAK1183H4 antibody molecule, e.g. scFv, IgG1, or IgG4.

One embodiment of the present disclosure comprises antibodies that neutralise naturally occurring IL-13 with a potency that is equal to or better than the potency of an IL-13 antigen-binding site formed by BAK1183H4 VH domain (SEQ ID NO: 2) and the BAK1183H4 VL domain (SEQ ID NO: 7).

Binding affinity and neutralisation potency of different antigen-binding proteins can be compared under appropriate conditions. Preferably, each of the binding affinity and neutralisation potency are measured under the same conditions for each antigen-binding protein (e.g., antibody).

When the antigen-binding protein of the disclosure is an antibody or an antigen-binding fragment thereof, it can further comprise a heavy chain immunoglobulin constant domain selected from the group consisting of:
(a) an IgA constant domain
(b) an IgD constant domain;
(c) an IgE constant domain;
(d) an IgG1 constant domain;
(e) an IgG2 constant domain;
(f) an IgG3 constant domain;
(g) an IgG4 constant domain; and
(h) an IgM constant domain.

The antigen-binding protein of the disclosure can further comprise a light chain immunoglobulin constant domain selected from the group consisting of:
(a) an Ig kappa constant domain; and
(b) an Ig lambda constant domain.

The antigen-binding protein of the disclosure can further comprise a human IgG1 constant domain and a human lambda constant domain.

The antigen-binding protein of the disclosure can comprise an IgG Fc domain containing a mutation at positions 252, 254 and 256, wherein the position numbering is according to the EU index as in Kabat. For example, the IgG1 Fc domain can contain a mutation of M252Y, S254T, and T256E, wherein the position numbering is according to the EU index as in Kabat.

The antigen-binding protein of the disclosure can bind a human IL-13 variant in which arginine at position 130 is replaced by glutamine or a human IL-13 variant in which arginine at position 105 is replaced by glutamine. Thus, antigen-binding proteins, e.g. antibodies, of the disclosure can recognize the human IL-13 variant, Q130R, which is associated with asthma, and/or the human IL-13 variant, Q105R. Cross-reactivity with variant IL-13 allows antibodies and antigen-binding fragments thereof of the present disclosure and compositions comprising antibodies and antigen-binding fragments thereof of the present disclosure to be used for the treatment of patients with wild-type and variant IL-13.

The antigen-binding protein of the disclosure can bind non-human primate IL-13, including rhesus and cynomolgus IL-13. Determining efficacy and safety profiles of an antibody or antigen-binding fragment thereof in non-human primates is extremely valuable as it provides a means for predicting the antibody or fragment's safety, pharmacokinetic, and pharmacodynamic profile in humans.

The antigen-binding protein or fragment thereof of the disclosure may bind an epitope comprising position 106 to C-terminal asparagine at position 132 (DTKIEVAQFVKDLLLHLKKLFREGRFN; SEQ ID NO:273) of human IL-13 protein. In one embodiment, the antigen-binding protein or fragment thereof binds an epitope comprising phenylalanine at position 99 to C-terminal asparagine at position 132 (FSSLHVRDTKIEVAQFVKDLLLHLKKLFREGRFN; SEQ ID NO:274) of human IL-13 protein.

The present disclosure also relates to an isolated VH domain of the antigen-binding protein of the disclosure and/or an isolated VL domain of the antigen-binding protein of the disclosure.

In addition to antibody sequences, an antigen-binding protein according to the present disclosure can comprise other amino acids, e.g. forming a peptide or polypeptide, such as a folded domain, or to impart to the molecule another functional characteristic in addition to ability to bind antigen. Antigen-binding proteins of the disclosure can carry a detectable label, or can be conjugated to a toxin or a targeting moiety or enzyme (e.g. via a peptidyl bond or linker).

A further aspect of the disclosure provides a method for obtaining an antibody or antigen-binding domain specific for human IL-13 antigen, the method comprising providing by way of addition, deletion, substitution, or insertion of one or more amino acids in the amino acid sequence of a VH domain set out herein, a VH domain which is an amino acid sequence variant of the VH domain, optionally combining the VH domain thus provided with one or more VL domains, and testing the VH domain or VH/VL combination or combinations to identify an antigen-binding protein or an antibody antigen-binding domain specific for IL-13 antigen and optionally with ability to neutralise IL-13 activity. Said VL domain can have an amino acid sequence which is substantially as set out herein.

An analogous method can be employed in which one or more sequence variants of a VL domain disclosed herein are combined with one or more VH domains.

In one embodiment, the BAK1183H4 VH domain (SEQ ID NO: 2) and/or the BAK1183H4 VL domain (SEQ ID NO: 7) can be subject to mutation to provide one or more VH domain and/or VL domain amino acid sequence variants.

A further aspect of the disclosure provides a method of preparing an antigen-binding protein specific for IL-13 antigen, which method comprises:
(a) providing a starting repertoire of nucleic acids encoding a VL domain disclosed herein, which either include a CDR3 to be replaced or lack a CDR3 encoding region;
(b) combining said repertoire with a donor nucleic acid encoding an amino acid sequence substantially as set out herein for a VL CDR3 such that said donor nucleic acid is inserted into the CDR3 region in the repertoire, so as to provide a product repertoire of nucleic acids encoding a VH domain;
(c) expressing the nucleic acids of said product repertoire;
(d) selecting an antigen-binding protein specific for IL-13 and which competes with a BAK1183H4 antibody for binding to IL-13;
(e) selecting an antigen-binding protein for IL-13 that binds to human IL-13 with an affinity better than that of the BAK1183H4 antibody, the affinity of the antigen-binding protein and the BAK1183H4 antibody being determined under the same conditions; and
(e) recovering said antigen-binding protein or nucleic acid encoding it.

Again, an analogous method can be employed in which a VH CDR3 of the disclosure is combined with a repertoire of nucleic acids encoding a VH domain which either include a CDR3 to be replaced or lack a CDR3 encoding region.

Similarly, one or more, or all three CDRs may be grafted into a repertoire of VH or VL domains which are then screened for an antigen-binding protein or antigen-binding proteins specific for IL-13, which compete with a BAK1183H4 antibody for binding to IL-13 and which bind to human IL-13 with an affinity better than that of the BAK1183H4 antibody, the affinity of the antigen-binding protein and the BAK1183H4 antibody being determined under the same conditions.

In one embodiment, one or more of 13NG0083 HCDR1 (SEQ ID NO: 13), HCDR2 (SEQ ID NO: 14) and HCDR3 (SEQ ID NO: 15) or the 13NG0083 set of HCDRs may be employed, and/or one or more of 13NG0083 LCDR1 (SEQ ID NO: 18), LCDR2 (SEQ ID NO: 19) and LCDR3 (SEQ ID NO: 20) or the 13NG0083 set of LCDRs.

A substantial portion of an immunoglobulin variable domain will comprise at least the three CDR regions, together with their intervening framework regions. The portion can also include at least about 50% of either or both of the first and fourth framework regions, the 50% being the C-terminal 50% of the first framework region and the N-terminal 50% of the fourth framework region. Additional residues at the N-terminal or C-terminal end of the substantial part of the variable domain may be those not normally associated with naturally occurring variable domain regions. For example, construction of antigen-binding proteins of the present disclosure made by recombinant DNA techniques may result in the introduction of N- or C-terminal residues encoded by linkers introduced to facilitate cloning or other manipulation steps. Other manipulation steps include the introduction of linkers to join variable domains of the disclosure to further protein sequences including immunoglobulin heavy chains, other variable domains (for example in the production of diabodies) or protein labels as discussed in more detail elsewhere herein.

Although in one aspect of the disclosure, antigen-binding proteins comprising a pair of VH and VL domains are envisaged, single binding domains based on either VH or VL domain sequences form further aspects of the disclosure. It is known that single immunoglobulin domains, especially VH domains, are capable of binding target antigens in a specific manner.

In the case of either of the single specific binding domains, these domains can be used to screen for complementary domains capable of forming a two-domain antigen-binding protein able to bind IL-13.

This can be achieved by phage display screening methods using the so-called hierarchical dual combinatorial approach as disclosed in WO92/01047, in which an individual colony containing either an H or L chain clone is used to infect a complete library of clones encoding the other chain (L or H) and the resulting two-chain antigen-binding protein is selected in accordance with phage display techniques such as those described in that reference. This technique is also disclosed in Marks et al., ibid.

Antigen-binding protein of the present disclosure can further comprise antibody constant regions or parts thereof. For example, a VL domain can be attached at its C-terminal end to antibody light chain constant domains including human Cκ or Cλ chains. Similarly, an antigen-binding protein based on a VH domain can be attached at its C-terminal end to all or part (e.g. a CH1 domain) of an immunoglobulin heavy chain derived from any antibody isotype, e.g. IgG, IgA, IgE and IgM and any of the isotype sub-classes, particularly IgG1 and IgG4. For example, the immunoglobulin heavy chain can be derived from the antibody isotype sub-class, IgG1. Any synthetic or other constant region variant that has these properties and stabilizes variable regions is also contemplated for use in embodiments of the present disclosure. The antibody constant region can be an Fc region with a YTE mutation, such that the Fc region comprises the following amino acid substitutions: M252Y/S254T/T256E. This residue numbering is based on Kabat numbering. The YTE mutation in the Fc region increases serum persistence of the antigen-binding protein (see Dall'Acqua, W. F. et al. (2006) The Journal of Biological Chemistry, 281, 23514-23524).

Antigen-binding proteins of the disclosure can be labelled with a detectable or functional label. Detectable labels include radiolabels such as ¹³¹I or ⁹⁹Tc, which may be attached to antibodies of the present disclosure using conventional chemistry known in the art of antibody imaging. Labels also include enzyme labels such as horseradish peroxidase. Labels further include chemical moieties such as biotin which may be detected via binding to a specific cognate detectable moiety, e.g. labelled avidin.

As noted, in various aspects and embodiments, the present disclosure extends to an antigen-binding protein or an antigen-binding fragment thereof which competes for binding to IL-13 with any antigen-binding protein defined herein, e.g. BAK1183H4. Competition between binding proteins can be assayed easily in vitro, for example by tagging a specific reporter molecule to one binding protein which can be detected in the presence of other untagged binding protein(s), to enable identification of antigen-binding proteins which bind the same epitope or an overlapping epitope.

Competition can be determined for example using ELISA in which IL-13 is immobilised to a plate and a first tagged binding member along with one or more other untagged binding members is added to the plate. Presence of an untagged binding member that competes with the tagged binding member is observed by a decrease in the signal emitted by the tagged binding member.

In testing for competition a peptide fragment of the antigen can be employed, especially a peptide including an epitope of interest. A peptide having the epitope sequence plus one or more amino acids at either end can be used. Such a peptide may be said to “consist essentially” of the specified sequence. Antigen-binding proteins according to the present disclosure can be such that their binding for antigen is inhibited by a peptide with or including the sequence given. In testing for this, a peptide with either sequence plus one or more amino acids may be used.

Antigen-binding proteins which bind a specific peptide can be isolated for example from a phage display library by panning with the peptide(s).

The antigen-binding protein of the disclosure can be capable of binding an epitope within the human IL-13 sequence from aspartic acid at position 106 to C-terminal asparagine at position 132 (DTKIEVAQFVKDLLLHLKKLFREGRFN; SEQ ID NO: 273) of human IL-13 protein. The antigen-binding protein of the disclosure can be capable of binding an epitope with the human IL-13 sequence from phenylalanine at position 99 to C-terminal asparagine at position 132 (FSSLHVRDTKIEVAQFVKDLLLHLKKLFREGRFN; SEQ ID NO: 274) of human IL-13 protein.

The present disclosure provides a method comprising causing or allowing binding of an antigen-binding protein as provided herein to IL-13. As noted, such binding can take place in vivo, e.g. following administration of an antigen-binding protein, or nucleic acid encoding an antigen-binding protein, or it may take place in vitro, for example in ELISA, Western blotting, immunocytochemistry, immuno-precipitation, affinity chromatography, or cell based assays such as a TF-1 assay.

The amount of binding of antigen-binding protein to IL-13 may be determined. Quantitation may be related to the amount of the antigen in a test sample, which may be of diagnostic interest.

### Methods of Treatment

Antigen-binding proteins of the present disclosure are designed to be used in methods of diagnosis or treatment in human or animal subjects.

Accordingly, further aspects of the disclosure provide methods of treatment comprising administration of an antigen-binding protein as provided, compositions (e.g. pharmaceutical compositions) comprising such an antigen-binding protein, and use of such an antigen-binding protein in the manufacture of a medicament for administration, for example in a method of making a medicament or pharmaceutical composition comprising formulating the antigen-binding protein with a pharmaceutically acceptable excipient.

Further aspects of the disclosure provide the antigen-binding protein of the disclosure for use in a method of treatment in a subject in need thereof, wherein the method comprises administration of said antigen-binding protein to said subject.

Clinical indications in which an anti-IL-13 antibody can be used to provide therapeutic benefit include asthma, chronic obstructive pulmonary disease (COPD), idiopathic pulmonary fibrosis (IPF), atopic dermatitis, allergic rhinitis, fibrosis, scleroderma, systemic sclerosis, pulmonary fibrosis, liver fibrosis, inflammatory bowel disease, ulcerative colitis, Sjögren's Syndrome and Hodgkin's lymphoma. As already explained, anti-IL-13 treatment is effective for all these diseases.

Antigen-binding proteins according to the disclosure can be used in a method of treatment or diagnosis of the human or animal body, such as a method of treatment (which may include prophylactic treatment) of a disease or condition in a human patient which comprises administering to said patient an effective amount of an antigen-binding protein of the disclosure. Diseases or conditions treatable in accordance with the present disclosure include any in which IL-13 plays a role, especially asthma, chronic obstructive pulmonary disease (COPD), idiopathic pulmonary fibrosis (IPF), atopic dermatitis, allergic rhinitis, fibrosis, scleroderma, systemic sclerosis, pulmonary fibrosis, liver fibrosis, inflammatory bowel disease, ulcerative colitis, Sjögren's Syndrome and Hodgkin's lymphoma. Further, the antibodies or antigen-binding fragments thereof of the present disclosure can also be used in treating tumours and viral infections as these antibodies and fragments will inhibit IL-13-mediated immunosuppression [64, 65].

Anti-IL-13 treatment can be given orally, by injection (for example, subcutaneously, intravenously, intraperitoneal or intramuscularly), by inhalation, or topically (for example intraocular, intranasal, rectal, into wounds, on skin). The route of administration can be determined by the physicochemical characteristics of the treatment, by special considerations for the disease or by the requirement to optimise efficacy or to minimise side-effects.

It is envisaged that anti-IL-13 treatment will not be restricted to use in the clinic. Therefore, subcutaneous injection using a needle free device is also envisaged.

Combination treatments can be used to provide significant synergistic effects, particularly the combination of an anti-IL-13 antigen-binding protein with one or more other drugs. An antigen-binding protein according to the present disclosure can be provided in combination or addition to short or long acting beta agonists, corticosteroids, cromoglycate, leukotriene (receptor) antagonists, methyl xanthines and their derivatives, IL-4 inhibitors, muscarinic receptor antagonists, IgE inhibitors, histaminic inhibitors, IL-5 inhibitors, eotaxin/CCR3 inhibitors, PDE4 inhibitors, TGF-beta antagonists, interferon-gamma, perfenidone, chemotherapeutic agents and immunotherapeutic agents.

Combination treatment with one or more short or long acting beta agonists, corticosteroids, cromoglycate, leukotriene (receptor) antagonists, xanthines, IgE inhibitors, IL-4 inhibitors, IL-5 inhibitors, eotaxin/CCR3 inhibitors, PDE4 inhibitors may be employed for treatment of asthma. Antibodies and antigen-binding fragments of the present disclosure can also be used in combination with corticosteroids, anti-metabolites, antagonists of TGF-beta and its downstream signalling pathway, for treatment of fibrosis. Combination therapy of these antibodies with PDE4 inhibitors, xanthines and their derivatives, muscarinic receptor antagonists, short and long beta antagonists can be useful for treating chronic obstructive pulmonary disease. Similar consideration of combinations apply to the use of anti-IL-13 treatment for atopic dermatitis, allergic rhinitis, chronic obstructive pulmonary disease, asthma, chronic obstructive pulmonary disease (COPD), idiopathic pulmonary fibrosis (IPF), atopic dermatitis, allergic rhinitis, fibrosis, scleroderma, systemic sclerosis, pulmonary fibrosis, liver fibrosis, inflammatory bowel disease, ulcerative colitis, Sjögren's Syndrome, and Hodgkin's lymphoma.

In accordance with the present disclosure, a method of treating, preventing, and/or ameliorating a disease or condition associated with IL-13 in a patient can comprise administration of an anti-IL-13 antibody or antigen-binding fragment as provided herein (e.g., an anti-IL-13 antibody or antigen-binding fragment as described in Tables 3-6 or FIG. 1-4, 15 or 17) and administration of an anti-IL-5R antibody or antigen-binding fragment thereof. In some embodiments, the anti-IL-5R antibody or antigen-binding fragment thereof is an anti-IL-5R antibody or antigen-binding fragment thereof described in U.S. Patent Application No. 2010/0291073 A1 and/or U.S. Pat. No. 6,018,032, each of which is incorporated herein by reference in its entirety. In additional embodiments, the anti-IL-5R antibody or antigen-binding fragment thereof is benralizumab or an antigen-binding fragment thereof. Information regarding benralizumab (or fragments thereof) for use in the methods provided herein can be found in U.S. Patent Application Publication No. 2010/0291073, the disclosure of which is incorporated herein by reference in its entirety. In additional embodiments, the anti-IL-5R antibody or antigen-binding fragment thereof comprises the HCDR1, HCDR2, and HCDR3 sequences of SEQ ID NOs: 280-282 and the LCDR1, LCDR2, and LCDR3 sequences of SEQ ID NOs: 283-285. In further embodiments, the anti-IL-5R antibody or antigen-binding fragment thereof comprises a VH domain comprising the sequence of SEQ ID NO: 278 or a VL domain comprising the sequence of SEQ ID NO: 276. In additional embodiments, the anti-IL-5R antibody or antigen-binding fragment thereof comprises a VH domain comprising the sequence of SEQ ID NO: 278 and a VL domain comprising the sequence of SEQ ID NO:276. In some embodiments, the anti-IL-5R antibody or antigen-binding fragment thereof comprises a heavy chain comprising the sequence of SEQ ID NO: 279, a light chain comprising the sequence of SEQ ID NO:277, or a heavy chain comprising the sequence of SEQ ID NO:279 and a light chain comprising the sequence of SEQ ID NO:277.

In accordance with the present disclosure, a method of treating, preventing, and/or ameliorating a disease or condition associated with IL-13 in a patient comprises administration of an anti-IL-13 antibody or antigen-binding fragment thereof and administration of an anti-IL-5R antibody or antigen-binding fragment thereof, wherein (i) the anti-IL-13 antibody or antigen-binding fragment thereof comprises a variable heavy domain comprising HCDR1, HCDR2, and HCDR3 sequences of SEQ ID NOs: 13-15 and a variable light domain comprising LCDR1, LCDR2, and LCDR3 sequences of SEQ ID NOs: 18-20 and (ii) the anti-IL-5R antibody or antigen-binding fragment thereof comprises a variable heavy domain comprising HCDR1, HCDR2, and HCDR3 sequences of SEQ ID NOs: 280-282 and a variable light domain comprising LCDR1, LCDR2, and LCDR3 sequences of SEQ ID NOs:283-285. In some embodiments, the anti-IL-13 antibody or antigen-binding fragment thereof comprises a variable heavy domain comprising the sequence of SEQ ID NO:12 and a variable light domain comprising the sequence of SEQ ID NO:17; and the anti-IL-5R antibody or antigen-binding fragment thereof comprises a variable heavy domain comprising HCDR1, HCDR2, and HCDR3 sequences of SEQ ID NOs: 280-282 and a variable light domain comprising LCDR1, LCDR2, and LCDR3 sequences of SEQ ID NOs:283-285. In additional embodiments, the anti-IL-13 antibody or antigen-binding fragment thereof comprises a variable heavy domain comprising HCDR1, HCDR2, and HCDR3 sequences of SEQ ID NOs: 13-15 and a variable light domain comprising LCDR1, LCDR2, and LCDR3 sequences of SEQ ID NOs: 18-20, and the anti-IL-5R antibody or antigen-binding fragment thereof comprises a heavy chain comprising the sequence of SEQ ID NO:278 and a light chain comprising the sequence of SEQ ID NO:276. In further embodiments, the anti-IL-13 antibody or antigen-binding fragment thereof comprises a variable heavy domain comprising the sequence of SEQ ID NO: 12 and a variable light domain comprising the sequence of SEQ ID NO: 17, and the anti-IL-5R antibody or antigen-binding fragment thereof comprises a heavy chain comprising the sequence of SEQ ID NO: 278 and a light chain comprising the sequence of SEQ ID NO:276.

In accordance with the present disclosure, a method of treating, preventing, and/or ameliorating a disease or condition associated with IL-13 in a patient can comprise administration of an anti-IL-13 antibody or antigen-binding fragment thereof and administration of an anti-IL-5R antibody or antigen-binding fragment thereof, wherein (i) the anti-IL-13 antibody or antigen-binding fragment thereof comprises a variable heavy domain comprising HCDR1, HCDR2, and HCDR3 sequences of SEQ ID NOs: 23-25 and a variable light domain comprising LCDR1, LCDR2, and LCDR3 sequences of SEQ ID NOs: 28-30 and (ii) the anti-IL-5R antibody or antigen-binding fragment thereof comprises a variable heavy domain comprising HCDR1, HCDR2, and HCDR3 sequences of SEQ ID NOs: 280-282 and a variable light domain comprising LCDR1, LCDR2, and LCDR3 sequences of SEQ ID NOs:283-285. In some embodiments, the anti-IL-13 antibody or antigen-binding fragment thereof comprises a variable heavy domain comprising the sequence of SEQ ID NO:22 and a variable light domain comprising the sequence of SEQ ID NO:27, and the anti-IL-5R antibody or antigen-binding fragment thereof comprises a variable heavy domain comprising HCDR1, HCDR2, and HCDR3 sequences of SEQ ID NOs: 280-282 and a variable light domain comprising LCDR1, LCDR2, and LCDR3 sequences of SEQ ID NOs:283-285. In additional embodiments, the anti-IL-13 antibody or antigen-binding fragment thereof comprises a variable heavy domain comprising HCDR1, HCDR2, and HCDR3 sequences of SEQ ID NOs: 23-25 and a variable light domain comprising LCDR1, LCDR2, and LCDR3 sequences of SEQ ID NOs: 28-30, and the anti-IL-5R antibody or antigen-binding fragment thereof comprises a heavy chain comprising the sequence of SEQ ID NO:278 and a light chain comprising the sequence of SEQ ID NO:276. In additional embodiments, the anti-IL-13 antibody or antigen-binding fragment thereof comprises a variable heavy domain comprising the sequence of SEQ ID NO:22 and a variable light domain comprising the sequence of SEQ ID NO:27, and the anti-IL-5R antibody or antigen-binding fragment thereof comprises a heavy chain comprising the sequence of SEQ ID NO:278 and a light chain comprising the sequence of SEQ ID NO:276.

In accordance with the present disclosure, a method of treating, preventing, and/or ameliorating a disease or condition associated with IL-13 in a patient can comprise administration of an anti-IL-13 antibody or antigen-binding fragment thereof and administration of an anti-IL-5R antibody or antigen-binding fragment thereof, wherein (i) the anti-IL-13 antibody or antigen-binding fragment thereof comprises a variable heavy domain comprising HCDR1, HCDR2, and HCDR3 sequences of SEQ ID NOs: 33-35 and a variable light domain comprising LCDR1, LCDR2, and LCDR3 sequences of SEQ ID NOs: 38-40 and (ii) the anti-IL-5R antibody or antigen-binding fragment thereof comprises a variable heavy domain comprising HCDR1, HCDR2, and HCDR3 sequences of SEQ ID NOs: 280-282 and a variable light domain comprising LCDR1, LCDR2, and LCDR3 sequences of SEQ ID NOs:283-285. In some embodiments, the anti-IL-13 antibody or antigen-binding fragment thereof comprises a variable heavy domain comprising the sequence of SEQ ID NO:32 and a variable light domain comprising the sequence of SEQ ID NO:37, and the anti-IL-5R antibody or antigen-binding fragment thereof comprises a variable heavy domain comprising HCDR1, HCDR2, and HCDR3 sequences of SEQ ID NOs: 280-282 and a variable light domain comprising LCDR1, LCDR2, and LCDR3 sequences of SEQ ID NOs:283-285. In additional embodiments, the anti-IL-13 antibody or antigen-binding fragment thereof comprises a variable heavy domain comprising HCDR1, HCDR2, and HCDR3 sequences of SEQ ID NOs: 33-35 and a variable light domain comprising LCDR1, LCDR2, and LCDR3 sequences of SEQ ID NOs: 38-40, and the anti-IL-5R antibody or antigen-binding fragment thereof comprises a heavy chain comprising the sequence of SEQ ID NO:278 and a light chain comprising the sequence of SEQ ID NO:276. In further embodiments, the anti-IL-13 antibody or antigen-binding fragment thereof comprises a variable heavy domain comprising the sequence of SEQ ID NO:32 and a variable light domain comprising the sequence of SEQ ID NO:37, and the anti-IL-5R antibody or antigen-binding fragment thereof comprises a heavy chain comprising the sequence of SEQ ID NO:278 and a light chain comprising the sequence of SEQ ID NO:276.

In accordance with the present disclosure, a method of treating, preventing, and/or ameliorating a disease or condition associated with IL-13 in a patient comprises administration of an anti-IL-13 antibody or antigen-binding fragment thereof provided herein (e.g., an anti-IL-13 antibody or antigen-binding fragment as described in Tables 3-6 or FIG. 1-4, 15 or 17) and administration of an anti-IL-5R antibody or antigen-binding fragment thereof provided herein, wherein the anti-IL-13 antibody or antigen-binding fragment thereof and the anti-IL-5R antibody or antigen-binding fragment thereof are administered concurrently (e.g., as part of the same composition or in separate compositions) or sequentially.

In accordance with the present disclosure, compositions provided may be administered to individuals. Administration is in a “therapeutically effective amount,” as defined above.

The precise dose will depend upon a number of factors, including whether the antibody or antigen-binding fragment thereof is for diagnosis or for treatment, the size and location of the area to be treated, the precise nature of the antibody (e.g. whole antibody, fragment or diabody), and the nature of any detectable label or other molecule attached to the antibody. A typical dose will be in the range 100 μg to 1 gm for systemic applications, and 1 μg to 1 mg for topical applications. Typically, the antibody will be a whole antibody, e.g. of the IgG4 isotype. This is a dose for a single treatment of an adult patient, which may be proportionally adjusted for children and infants, and also adjusted for other antibody formats in proportion to molecular weight. Treatments can be repeated at daily, twice-weekly, weekly or monthly intervals, at the discretion of the physician. In some embodiments of the present disclosure, treatment is periodic, and the period between administrations is about two weeks or more, about three weeks or more, about four weeks or more, or about once a month.

Antigen-binding proteins of the present disclosure will usually be administered in the form of a pharmaceutical composition, which can comprise at least one component in addition to the antigen-binding protein.

Thus pharmaceutical compositions according to the present disclosure, and for use in accordance with the present disclosure, can comprise, in addition to active ingredient, a pharmaceutically acceptable excipient, vehicle, carrier, buffer, stabiliser or other materials well known to those skilled in the art. Such materials should be non-toxic and should not interfere with the efficacy of the active ingredient. The precise nature of the carrier or other material will depend on the route of administration, which may be oral, or by injection, e.g. intravenous.

Thus, the disclosure also provides a pharmaceutical composition comprising the antigen-binding protein of the disclosure and a pharmaceutically acceptable excipient.

Pharmaceutical compositions for oral administration may be in tablet, capsule, powder or liquid form. A tablet may comprise a solid carrier such as gelatin or an adjuvant. Liquid pharmaceutical compositions generally comprise a liquid carrier such as water, petroleum, animal or vegetable oils, mineral oil or synthetic oil. Physiological saline solution, dextrose or other saccharide solution or glycols such as ethylene glycol, propylene glycol or polyethylene glycol may be included.

For intravenous injection, or injection at the site of affliction, the active ingredient will be in the form of a parenterally acceptable aqueous solution which is pyrogen-free and has suitable pH, isotonicity and stability. Those of relevant skill in the art are well able to prepare suitable solutions using, for example, isotonic vehicles such as Sodium Chloride Injection, Ringer's Injection, Lactated Ringer's Injection. Preservatives, stabilisers, buffers, antioxidants and/or other additives may be included, as required.

A composition can be administered alone or in combination with other treatments, either simultaneously or sequentially dependent upon the condition to be treated. For example, a composition comprising an anti-IL-13 antibody or antigen-binding fragment provided herein (e.g., an anti-IL-13 antibody or antigen-binding fragment as described in Tables 3-6 or FIG. 1-4, 15 or 17) can be administered alone or in combination with an anti-IL-5R antibody or antigen-binding fragment (e.g., benralizumab or an antigen-binding fragment thereof), either simultaneously (concurrently) or sequentially.

Antigen-binding proteins of the present disclosure can be formulated in liquid or solid forms depending on the physicochemical properties of the molecule and the route of delivery. Formulations can include excipients, or combinations of excipients, for example: sugars, amino acids and surfactants. Liquid formulations may include a wide range of antibody concentrations and pH. Solid formulations may be produced by lyophilisation, spray drying, or drying by supercritical fluid technology, for example. Formulations of anti-IL-13 will depend upon the intended route of delivery: for example, formulations for pulmonary delivery may consist of particles with physical properties that ensure penetration into the deep lung upon inhalation; topical formulations may include viscosity modifying agents, which prolong the time that the drug is resident at the site of action.

The pharmaceutical composition of the disclosure can further comprise a labeling group or an effector group. For example, the labeling group may be selected from the group consisting of: an isotopic label, a magnetic label, a redox active moiety, an optical dye, a biotinylated group and a polypeptide epitope recognized by a secondary reporter, such as GFP or biotin. The effector group may, for example, be selected from the group consisting of a radioisotope, radionuclide, a toxin, a therapeutic and a chemotherapeutic agent.

In some embodiments, a pharmaceutical composition comprises an anti-IL-13 antibody or antigen-binding fragment thereof provided herein (e.g., an anti-IL-13 antibody or antigen-binding fragment as described in Tables 3-6 or FIG. 1-4, 15 or 17) and an anti-IL-5R antibody or antigen-binding fragment thereof provide herein (e.g., benralizumab or an antigen-binding fragment thereof or an anti-IL-5R antibody or fragment thereof described in U.S. Patent Application Publication No. 2010/0291073, herein incorporated by reference in its entirety).

In some aspects of the present disclosure, a subject is a naïve subject. A naïve subject is a subject that has not been administered a therapy, for example a therapeutic agent. In some aspects, a naïve subject has not been treated with a therapeutic agent prior to being diagnosed as having an IL-13-mediated disease or condition, for example, asthma, IFP, COPD, Atopic dermatitis, or UC. In another aspect, a subject has received therapy and/or one or more doses of a therapeutic agent (e.g., a therapeutic agent capable of modulating an inflammatory response associated with an IL-13-mediated disease or condition, a pulmonary disease or condition, a chronic inflammatory skin condition, or an inflammatory bowel disease or condition) prior to being diagnosed as having an IL-13-mediated disease or condition. In one aspect, the therapeutic agent is a small molecule drug. In a specific aspect, the agent is a corticosteroid. In another aspect, the agent can be a leukotriene modifier such as montelukast, zafirlukast or zileuton. In a further aspect, the therapeutic agent can be a methylxanthine (e.g., theophylline) or a cromone (e.g., sodium cromolyn and nedocromil). In another aspect, the therapeutic agent can be a long-acting beta-2 agonist such as salmeterol, fomoterol, or indacaterol. In a further aspect, the agent can be methotrexate or cyclosporin.

In certain aspects, the therapeutic agent can be an agent used for preventing, treating, managing, or ameliorating asthma. Non-limiting examples of therapies for asthma include anti-cholinergics (e.g., ipratropium bromide and oxitropium bromide), beta-2 antagonists (e.g., albuterol (PROVENTIL® or VENTOLIN®), bitolterol (TOMALATE®), fenoterol, formoterol, isoetharine, metaproterenol, pibuterol (MAXAIR®), salbutamol, salbutamol terbutaline, and salmeterol, terbutlaine (BRETHAIRE®)), corticosteroids (e.g., prednisone, beclomethasone dipropionate (VANCERIL® or BECLOVENT®), triamcinolone acetonide (AZMACORF®), flunisolide (AEROBID®), and fluticasone propionate (FLOVENT®)), leukotriene antagonists (e.g., montelukast, zafirlukast, and zileuton), theophylline (THEO-DUR®, UNIDUR® tablets, and SLO-BID® Gyrocaps), and salmeterol (SEREVENT®), cromolyn, and nedorchromil (INTAL® and TILADE®)), IgE antagonists, IL-4 antagonists (including antibodies), IL-5 antagonists (including antibodies), PDE4 inhibitors, NF-Kappa-B inhibitors, IL-13 antagonists (including antibodies), CpG, CD23 antagonists, selectin antagonist (e.g., TBC 1269), mast cell protease inhibitors (e.g., tryptase kinase inhibitors (e.g., GW-45, GW-58, and genisteine), phosphatidylinositide-3′ (PI3)-kinase inhibitors (e.g., calphostin C), and other kinase inhibitors (e.g., staurosporine), C2a receptor antagonists (including antibodies), and supportive respiratory therapy, such as supplemental and mechanical ventilation.

In some aspects, a subject has received at least one therapeutically effective dose of oral or inhaled corticosteroids. In some aspects, a subject has received multiple therapeutically effective doses of oral or inhaled corticosteroids. In some aspects, a subject is a chronic oral corticosteroid (OCS) user.

In certain aspects the subject has received a long-acting beta2-adrenergic agonist, e.g., salmeterol xinafoate. In some aspects the subject has received a synthetic glucocorticoid, e.g., fluticasone propionate. In certain aspects the subject has received a combination of salmeterol xinafoate and fluticasone propionate (ADVAIR®). In certain aspects the subject has received a beta2-adrenergic bronchodilator, e.g., albuterol sulfate.

### Kits

A kit comprising an isolated antigen-binding protein (e.g. an antibody molecule or antigen-binding fragment thereof) according to any aspect or embodiment of the present disclosure is also provided as an aspect of the present disclosure. In a kit, the antigen-binding protein or antibody molecule can be labelled to allow its reactivity in a sample to be determined, e.g. as described further below. Components of a kit are generally sterile and in sealed vials or other containers. Kits can be employed in diagnostic analysis or other methods for which antibody molecules are useful. A kit can contain instructions for use of the components in a method, e.g. a method in accordance with the present disclosure. Ancillary materials to assist in or to enable performing such a method may be included within a kit of the disclosure.

The reactivities of antibodies in a sample can be determined by any appropriate means. Radioimmunoassay (RIA) is one possibility. Radioactive labelled antigen is mixed with unlabelled antigen (the test sample) and allowed to bind to the antibody. Bound antigen is physically separated from unbound antigen and the amount of radioactive antigen bound to the antibody determined. The more antigen there is in the test sample the less radioactive antigen will bind to the antibody. A competitive binding assay can also be used with non-radioactive antigen, using antigen or an analogue linked to a reporter molecule. The reporter molecule can be a fluorochrome, phosphor or laser dye with spectrally isolated absorption or emission characteristics. Suitable fluorochromes include fluorescein, rhodamine, phycoerythrin and Texas Red. Suitable chromogenic dyes include diaminobenzidine.

Other reporters include macromolecular colloidal particles or particulate material such as latex beads that are coloured, magnetic or paramagnetic, and biologically or chemically active agents that can directly or indirectly cause detectable signals to be visually observed, electronically detected or otherwise recorded. These molecules can be enzymes which catalyse reactions that develop or change colours or cause changes in electrical properties, for example. They can be molecularly excitable, such that electronic transitions between energy states result in characteristic spectral absorptions or emissions. They can include chemical entities used in conjunction with biosensors. Biotin/avidin or biotin/streptavidin and alkaline phosphatase detection systems can be employed.

The signals generated by individual antibody-reporter conjugates can be used to derive quantifiable absolute or relative data of the relevant antibody binding in samples (normal and test).

The present disclosure also provides the use of an antigen-binding protein as above for measuring antigen levels in a competition assay, that is to say a method of measuring the level of antigen in a sample by employing an antigen-binding protein as provided by the present disclosure in a competition assay. This can be where the physical separation of bound from unbound antigen is not required. Linking a reporter molecule to the antigen-binding protein so that a physical or optical change occurs on binding is one possibility. The reporter molecule can directly or indirectly generate detectable, and preferably measurable, signals. The linkage of reporter molecules may be directly or indirectly, covalently, e.g. via a peptide bond or non-covalently. Linkage via a peptide bond can be as a result of recombinant expression of a gene fusion encoding antibody and reporter molecule.

The present disclosure also provides for measuring levels of antigen directly, by employing an antigen-binding protein according to the disclosure for example in a biosensor system.

The mode of determining binding is not a feature of the present disclosure, and those skilled in the art are able to choose a suitable mode according to their preference and general knowledge.

### Polynucleotides and Host Cells

In further aspects, the present disclosure provides an isolated nucleic acid which comprises a sequence encoding an antigen-binding protein, VH domain and/or VL domain according to the present disclosure, and methods of preparing an antigen-binding protein, a VH domain and/or a VL domain of the disclosure, which comprise expressing said nucleic acid under conditions to bring about production of said antigen-binding protein, VH domain and/or VL domain, and recovering it.

Nucleic acid includes DNA and/or RNA. In one aspect, the nucleic acid is cDNA. In one aspect, the present disclosure provides a nucleic acid which codes for a CDR or set of CDRs or VH domain or VL domain or antibody antigen-binding site or antibody molecule, e.g. scFv or IgG1, of the disclosure as defined above.

One aspect of the present disclosure provides nucleic acid, generally isolated, optionally a cDNA, encoding a VH CDR or VL CDR sequence disclosed herein, especially a VH CDR selected from SEQ ID NOs: 13-15 or a VL CDR selected from SEQ ID NOs: 18-20. Nucleic acid encoding the 13NG0083 set of CDRs, nucleic acid encoding the 13NG0083 set of HCDRs and nucleic acid encoding the 13NG0083 set of LCDRs are also provided, as are nucleic acids encoding individual CDRs, HCDRs, LCDRs and sets of CDRs, HCDRs, LCDRs of the BAK1183H4 lineage.

The present disclosure provides an isolated polynucleotide or cDNA molecule sufficient for use as a hybridization probe, PCR primer or sequencing primer that is a fragment of a nucleic acid molecule disclosed herein or its complement. The nucleic acid molecule can, for example, be operably linked to a control sequence.

The present disclosure also provides constructs in the form of plasmids, vectors, transcription or expression cassettes which comprise at least one polynucleotide as above.

The present disclosure also provides a recombinant host cell which comprises one or more constructs as above. A nucleic acid encoding any CDR or set of CDRs or VH domain or VL domain or antibody antigen-binding site or antibody molecule, e.g. scFv or IgG1 as provided, itself forms an aspect of the present disclosure, as does a method of production of the encoded product, which method comprises expression from encoding nucleic acid therefor. Expression can conveniently be achieved by culturing under appropriate conditions recombinant host cells containing the nucleic acid. Following production by expression a VH or VL domain, or an antigen-binding protein may be isolated and/or purified using any suitable technique, then used as appropriate.

The host cell can be a mammalian host cell, such as a NS0 murine myeloma cell, a PER.C6® human cell, or a Chinese hamster ovary (CHO) cell.

Antigen-binding proteins, VH and/or VL domains, and encoding nucleic acid molecules and vectors can be isolated and/or purified, e.g. from their natural environment, in substantially pure or homogeneous form, or, in the case of nucleic acid, free or substantially free of nucleic acid or genes origin other than the sequence encoding a polypeptide with the required function. Nucleic acid according to the present disclosure may comprise DNA or RNA and can be wholly or partially synthetic. Reference to a nucleotide sequence as set out herein encompasses a DNA molecule with the specified sequence, and encompasses a RNA molecule with the specified sequence in which U is substituted for T, unless context requires otherwise.

Systems for cloning and expression of a polypeptide in a variety of different host cells are well known. Suitable host cells include bacteria, mammalian cells, plant cells, yeast and baculovirus systems and transgenic plants and animals. Mammalian cell lines available in the art for expression of a heterologous polypeptide include Chinese hamster ovary (CHO) cells, HeLa cells, baby hamster kidney cells, NS0 mouse melanoma cells, YB2/0 rat myeloma cells, human embryonic kidney cells, human embryonic retina cells and many others. A common bacterial host is E. coli.

The expression of antibodies and antibody fragments in prokaryotic cells such as E. coli is well established in the art. For a review, see for example Plückthun, A. Bio/Technology 9: 545-551 (1991). Expression in eukaryotic cells in culture is also available to those skilled in the art as an option for production of an antigen-binding protein for example Chadd H E and Chamow S M (2001) 110 Current Opinion in Biotechnology 12: 188-194, Andersen D C and Krummen L (2002) Current Opinion in Biotechnology 13: 117, Larrick J W and Thomas D W (2001) Current opinion in Biotechnology 12:411-418.

Suitable vectors can be chosen or constructed, containing appropriate regulatory sequences, including promoter sequences, terminator sequences, polyadenylation sequences, enhancer sequences, marker genes and other sequences as appropriate. Vectors may be plasmids, viral e.g. ‘phage, or phagemid, as appropriate. For further details see, for example, Molecular Cloning: a Laboratory Manual: 3rd edition, Sambrook and Russell, 2001, Cold Spring Harbor Laboratory Press. Many known techniques and protocols for manipulation of nucleic acid, for example in preparation of nucleic acid constructs, mutagenesis, sequencing, introduction of DNA into cells and gene expression, and analysis of proteins, are described in detail in Current Protocols in Molecular Biology, Second Edition, Ausubel et al. eds., John Wiley & Sons, 1988, Short Protocols in Molecular Biology: A Compendium of Methods from Current Protocols in Molecular Biology, Ausubel et al. eds., John Wiley & Sons, 4th edition 1999. The disclosures of Sambrook et al. and Ausubel et al. (both) are incorporated herein by reference.

Thus, a further aspect of the present disclosure provides a host cell containing nucleic acid as disclosed herein. For example, the disclosure provides a host cell transformed with nucleic acid comprising a nucleotide sequence encoding an antigen-binding protein of the disclosure or antibody VH or VL domain of an antigen-binding protein of the disclosure.

Such a host cell can be in vitro and can be in culture. Such a host cell can be an isolated host cell. Such a host cell can be in vivo. In vivo presence of the host cell can allow intracellular expression of the antigen-binding proteins of the present disclosure as “intrabodies” or intracellular antibodies. Intrabodies can be used for gene therapy [74].

A still further aspect provides a method comprising introducing such nucleic acid into a host cell. The introduction can employ any available technique. For eukaryotic cells, suitable techniques may include calcium phosphate transfection, DEAE-Dextran, electroporation, liposome-mediated transfection and transduction using retrovirus or other virus, e.g. vaccinia or, for insect cells, baculovirus. Introducing nucleic acid in the host cell, in particular a eukaryotic cell can use a viral or a plasmid based system. The plasmid system can be maintained episomally or may incorporated into the host cell or into an artificial chromosome [72,73]. Incorporation can be either by random or targeted integration of one or more copies at single or multiple loci. For bacterial cells, suitable techniques may include calcium chloride transformation, electroporation, and transfection using bacteriophage.

The introduction can be followed by causing or allowing expression from the nucleic acid, e.g. by culturing host cells under conditions for expression of the gene.

In one embodiment, the nucleic acid of the present disclosure is integrated into the genome (e.g. chromosome) of the host cell. Integration can be promoted by inclusion of sequences which promote recombination with the genome, in accordance with standard techniques.

The present disclosure also provides a method which comprises using a construct as stated above in an expression system in order to express an antigen-binding protein or polypeptide as above.

In another aspect, the disclosure provides a hybridoma producing the antigen-binding protein of the disclosure.

A yet further aspect of the disclosure provides a method of production of an antibody binding protein of the disclosure, the method including causing expression from encoding nucleic acid. Such a method can comprise culturing host cells under conditions suitable for production of said antigen-binding protein.

Analogous methods for production of VH and VL variable domains are provided as further aspects of the present disclosure.

A method of production can comprise a step of isolation and/or purification of the product from the host cell or hybridoma.

A method of production can comprise formulating the product into a composition including at least one additional component, such as a pharmaceutically acceptable excipient.

Aspects and embodiments of the present disclosure will now be illustrated by way of example with reference to the following experimentation.

### EXAMPLES

### Example 1

### Generation of Antibody Clones that Bind Human IL-13 with an Affinity Better than that of the BAK1183H4 Antibody

A number of anti-IL-13 antibodies are currently being developed as therapies for treatment of patients with IL-13 related diseases or conditions, such as moderate to severe asthma. These antibodies include: Lebrikizumab (MILR1444A/RG3637, Roche/Genentech), ABT-308 (Abbott), GSK679586 (GlaxoSmithKline), QAX576 (Novartis), and Tralokinumab (CAT-354, MedImmune/AstraZeneca). Although the effectiveness of these therapeutics is encouraging, there remains a need for improved anti-IL-13 antibodies having higher affinity and increased serum persistence or half-life to increase efficacy and reduce frequency of administration.

Some anti-IL-13 antibodies currently in clinical development have an affinity for human IL-13 of approx. 100-200 pM. Modelling indicated that a KD less than 10 pM (i.e. higher affinity) combined with increased serum persistence could provide significant clinical benefit.

Anti-human IL-13 antibody clone 1183H04 (also referred to herein as “1183H4” or “BAK1183H4”) was generated in an affinity maturation campaign involving phage display and ribosome display described previously (see, e.g., Thom et al., 2006; PNAS 103 p 7619-7624; WO 2005/007699 and U.S. Pat. No. 7,829,090). 1183H04 affinity to human IL-13 was measured by BIAcore to be 81 pM.

The optimisation campaign utilized to generate clone 1183H04 was extensive, in terms of CDR loops targeted for mutagenesis. There was, therefore, limited sequence space left to explore for further affinity gains. This secondary affinity maturation strategy therefore involved primarily targeting the light chain variable regions with NNS codon mutagenesis in blocks of up to amino acids in one go, and also included so-called Vernier residues (Ref: Foote and Winter (1992) J. Mol. Biol. 224 p 487-499) in the hope of achieving additional affinity improvements. A summary of the residues targeted is shown in Table 1.

TABLE 1
Summary of residues targeted in 1183H04
affinity maturation campaign.

| | | | FW2 | | | | FW4 |
|---|---|---|---|---|---|---|---|
| | | | (Ver- | | | | (Ver- |
| VL | FW1 | CDR1 | nier) | CDR2 | Fw3 | CDR3 | nier) |
| Targeted | | 27-31, | 35-36 | 51-56 | | 89-90 | 98 |
| residues | | 33 | 46-49 | | | 92-94 | |
| | | | | | | 95a, | |
| | | | | | | 95b, | |
| | | | | | | 96-97 | |
| Protected | | 32, 34 | | 50 | | 91, 95 | |
| residues* | | | | | | | |

*based on alanine scanning data (Thom et al., 2006)

In addition, the VH CDR1 residues 30-35 and Vernier residues in FW1 27-30 were targeted as part of the randomisation strategy.

Residues that had been previously shown to be critical for binding, by alanine scanning, were not randomised if they were present within a block.

Amino acid randomisation was performed using oligo directed mutagenesis, and phage display libraries were prepared for selections following sequence QC. (All libraries generated were >1e9 which is sufficiently high to cover the theoretical diversity for a block of 6 amino acids, using this mutagenesis strategy.)

Solution-phase selections (3-4 rounds) using phage display with decreasing concentrations (10-0.1 nM) of biotinylated recombinant IL-13 were performed (IL-13 (Peprotech) was biotinylated in house). Individual scFvs were screened as crude supernatants in a biochemical receptor-ligand inhibition assay, looking for those that inhibited to a greater degree than the parental 1183H04 scFv (“hits”). Hits were then screened and ranked as purified scFv or IgG in the biochemical assay and/or the biological TF-1 assay.

Sequence diversity was limited to only a small number of residues within each of the targeted blocks (at best 3-4). This suggested that the sequence areas targeted were relatively intolerant to changes.

Interestingly, the VL CDR3 only tolerated a single mutation within the six randomised residues. This was at position 95a, and only 3 possible amino acids (R, S, or L) were found in place of the parental (D) residue. The original libraries had all shown good diversity at all randomised positions.

After 2-3 rounds of phage display selection, outputs were selected for preparing recombination libraries, in order to select for combinations of mutations that conferred additive or synergistic improvements. Libraries were constructed by combining H1 with L1B1, L2, L3, and all combinations within to generate a total of 6 phage display recombination libraries (all >1e9 in size following transformations).

Phage display solution-phase selections were performed once again with decreasing concentration of Bio-IL-13 (1-0.01 nM), and at R3 competitive selections were performed using an excess of unbiotinylated IL-13. Outputs were screened in the receptor ligand inhibition assay from R1-R4 post recombination as crude scFv. Hits were prepared as purified scFv or IgG material and were tested in the biological assay.

TABLE 2
Summary of number of clones screened.

| | | | | | Post- | |
|---|---|---|---|---|---|---|
| | | | | | recombination | |
| | | | | Post- | min library | |
| | | | | recombination | error prone | |
| | | Pre- | Post- | mini library | ribosome display | |
| | Format | recombination | recombincation | (CDR L1, 2, 3) | (CDR L1, 2, 3) | Total |
| Number of | Unpurified | 3872 | 3168 | 991 | 1408 | 9439 |
| clones | ScFv | | | | | |

| | | | | | |
|---|---|---|---|---|---|
| screened | Purified | 20 | 32 | 22 | 74 |
| | ScFv | | | | |
| | IgG | 4 | 9 | 22 | 35 |

Once again a modest number of hits with improved IC50 over parent were generated in the assay. Table 2 above shows the number of clones screened during the optimisation process and the format in which they were screened. Despite screening large numbers of variants as crude scFv, relatively few (less than 0.8% or 74/9439) showed improvements over parent and were taken forward for further characterisation as purified scFv or IgG.

There was some difficulty throughout the optimisation process in ranking the improved variants using the biochemical and biological assays as the sequence differences were relatively conservative and the improvements in IC50 were difficult to differentiate.

Affinity data using a Biacore affinity assay and then Kinexa, on a limited subset, facilitated the ranking of the variants and was used throughout the optimisation process, to monitor affinity improvements.

The greatest affinity improvements from the recombination libraries were observed by combining VL CDR1 and VL CDR3 or VLCDR2 with VLCDR3. To investigate whether the affinity could be improved further a ‘mini-library’ was constructed to recombine mutations in VL CDR1, CDR2, and CDR3. These mutations were shuffled using PCR with overlapping primers. The estimated diversity at this stage was ˜288 possible combinations so rather than performing further selections a population of approximately 1000 colonies was picked directly from the mini-library transformation plates and screened directly in the biochemical assay.

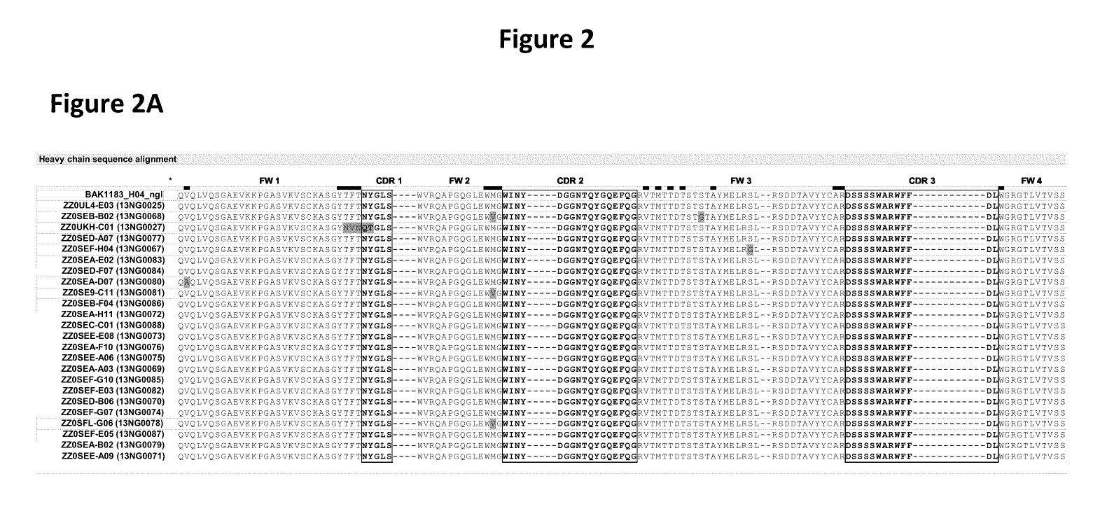

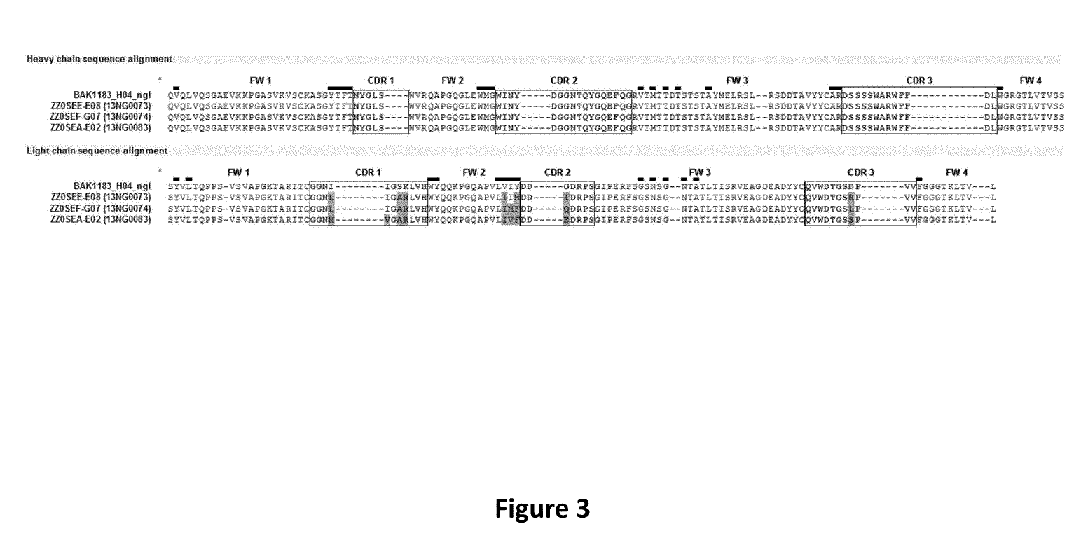

242 scFvs of the ˜1000 assayed showed greater potency than 1183H04 in a competition assay. These were sequenced and showed recombination of VLCDRs 1, 2, and 3. 33 unique variants were selected, based on sequence diversity, to be screened as purified scFvs in biochemical and biological assays. The 22 most potent hits in the biochemical assay were selected to be prepared as IgG for ranking in the biology assay (see FIGS. 1a, 1b, 2a, and 2b). The top 4-5 IgGs from the biological assays were ranked in a Biacore affinity assay. The top 3 clones, including 13NG0073, 13NG0074 and 13NG0083 (FIG. 3), in the Biacore affinity assay were chosen for analysing affinity gains using Kinexa.

The mini-library was also subjected to error prone PCR and several rounds of ribosome display but this did not produce any further improvements in the potency.

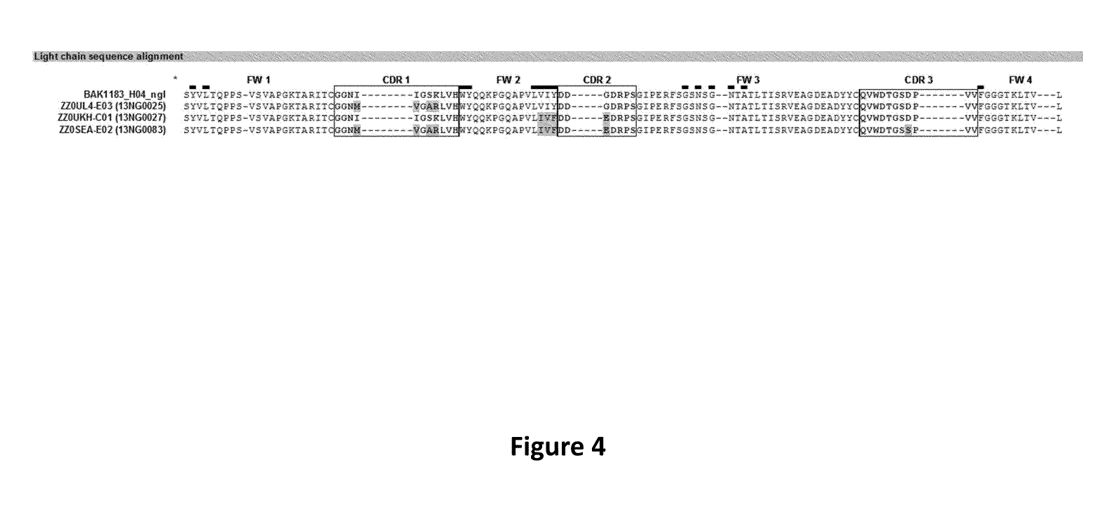

The optimisation of 1183H04 was a challenging process especially as this variant had been the product of an extensive optimisation campaign. See, e.g., U.S. Pat. No. 7,829,090. It was not clear that the desired affinity target during this current optimisation process was achievable. Sequence changes in variants from pre- and post-recombination selections were minor and generated only modest improvements in affinity. FIG. 4 shows two variants from the pre recombination selections that had been screened in the biochemical and biological assays (13NG0025 and 13NG0027). The individual clones had only modest improvements over the parent in the assays. Surprisingly, combining the changes from these variants, together with an additional mutation at position 95a in the VLCDR3, generated an unexpected, 5.2 fold improvement in affinity to a Kd of 6 pM (13NG0083).

### Example 2

### Potency of Clone 13NG0083 in a TF1 Proliferation Assay

Clone 13NG0083 potency was tested in a TF1 cell proliferation assay. Briefly, TF1 cells (R&D Systems) were washed and re-suspended in assay media to a final concentration of 2×10⁵/mL [Assay media: RPMI-1640 (Gibco), 5% Foetal Bovine Serum, lx Penicillin/Streptomycin (Gibco)]. One hundred microliters of cells were dispensed into a 96-well flat-bottomed assay plate (Costar). Human interleukin 13 (Peprotech) diluted to a concentration of 40 ng/mL was dispensed into a separate assay plate. A titration range of 13NG0083 (IgG1 format with a YTE mutation in the Fc region) or isotype control, was prepared at four times final concentration in a separate assay plate. Equal volumes of the antibody and IL-13 were then mixed and incubated for 30 minutes at room temperature. All dilutions of cells, ligand and antibodies were made in assay media. One hundred microliters of the antibody/IL-13 combination was then added to the TF1 cells. Cells with media alone or IL-13 alone were used as negative or positive controls respectively. Cells were cultured for 3 days at 37° C., 5% CO₂. After culture period cells were pulsed with 20 microliters/well of [3H]-Thymidine (Perkin-Elmer). Cells were incubated for four hours at 37° C., 5% CO₂ and then harvested on to glass fibre filter plates (Perkin-Elmer) and dried for 1 hour at 50° C. Fifty microliters/well of Microscint (Perkin-Elmer) was added, plates sealed and read on a scintillation counter. Results were expressed as counts per minute (C.P.M.).

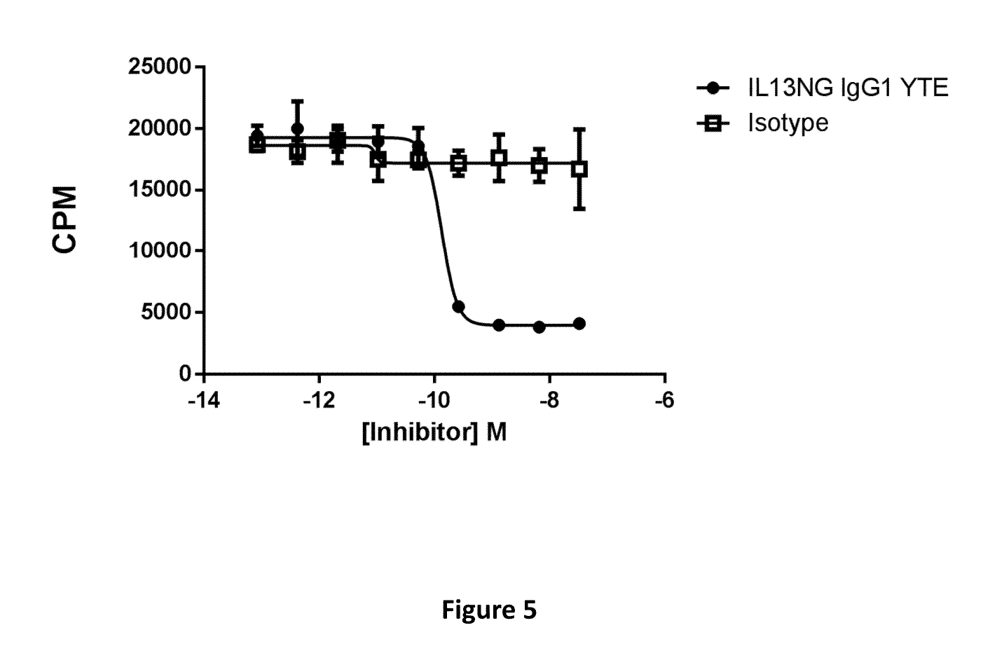

The experiments were performed three times to assess potency of the antibody 13NG0083 (IgG1 format with a YTE mutation in the Fc region). FIG. 5 shows a representative single experiment showing that 13NG0083 (IgG format with a YTE mutation in the Fc region) potently inhibits TF1 proliferation. Data was plotted as C.P.M. versus log(10) concentration of antibody and fitted to a Sigmoidal dose response model (variable slope) Y=Bottom+(Top−Bottom)/(1+10̂((Log EC50−X)*HillSlope)) where; X is the logarithm of concentration. Y is the response; Y starts at Bottom and goes to Top with a sigmoid shape. This is the “four parameter logistic equation. Data analysis was performed using Microsoft Excel and Graphpad Prism software. IC50 values were obtained from three independent experiments which gave a geometric mean IC50 value of 165 pM (95% CI of geometric mean; 26-1052 pM).

### Example 3

### Potency of 13NG0083 in a Receptor Ligand Competition Assay Using the R130 Variant of IL-13

13NG0083 variants were tested for their ability to inhibit IL-13 binding to IL-13 Receptorα2 using Homogenous Time Resolved Fluorescence (HTRF). Briefly, an HTRF assay was developed whereby a FRET signal was seen when FLAG-tagged human IL-13 (detected with a Europium-labelled anti-FLAG antibody (CisBio)) bound to human IL-13 Receptorα2 (R&D systems) that had been previously directly labelled with Dylight650 (Thermo Scientific). Final assay conditions were as follows: Anti-FLAG Europium cryptate (433 pM), FLAG-tagged human IL-13 (312.5 pM), and human IL-13 Receptorα2 (10 nM) were added to a black shallow-384-well plate(Costar), sealed, covered, and incubated at room temperature for 4 hours. Plates were then read using an Envision microplate reader (PerkinElmer) using a 320 nm excitation filter and 590 nm and 665 nm emission filters. Ratios for the emission values seen at 665 nm and 620 nm were calculated using the following formula, (665/620)*10,000. Finally DeltaF values were calculated using the following formula ((Test well ratio−non-specific background ratio)/non-specific background ratio)*100. Non-specific background was defined as the HRTF signal seen in control wells (typically wells 123 to P24 inclusive) where the addition of FLAG-tagged human IL-13 was omitted and replaced with assay buffer.

In order to determine the potency of 13NG0083 variants at inhibiting the interaction of human IL-13 and IL-13 receptorα2, 11-point dose response experiments were performed with concentrations of variants in duplicate. These titrations were added to the above HTRF competition assay and the data fitted with to a Sigmoidal dose response model (variable slope) Y=Bottom (Top−Bottom)/(1+10̂((Log EC50−X)*HillSlope)) where; X is the logarithm of concentration. Y is the response; Y starts at Bottom and goes to Top with a sigmoid shape. This is the “four parameter logistic equation. Data analysis was performed using Microsoft Excel and Graphpad Prism software.

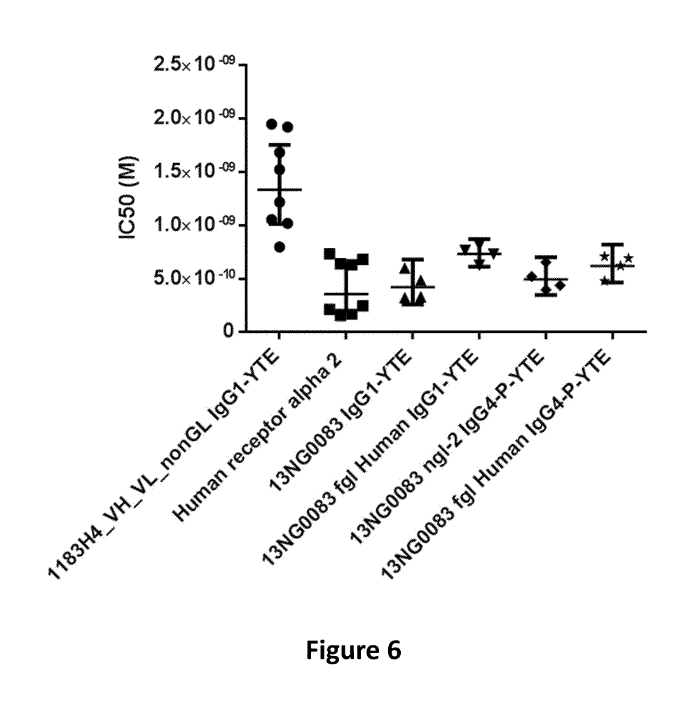

The results of these experiments are shown in FIG. 6 and show a significant improvement in the geometric mean potency from the parent clone (IC₅₀=1.34 nM) to the optimised variants with little effect seen with altering the format of the 13NG0083 clone from IgG1 (13NG0083 IgG1 IC₅₀=423 pM) to IgG4-P (13NG0083 IgG4-P IC₅₀=496 pM), nor upon changes to germline (13NG0083 IgG1 FGL IC₅₀=734 pM & 13NG0083 IgG4-P IC₅₀=622 pM). IgG4-P: IgG4 S241P.

### Example 4

### Affinity of the 13NG0073, 13NG0074 and 13NG0083 Clones

### Methods

### Materials/Reagents/Chemicals

Azlactone beads were from (Thermo), Dulbecco's PBS.

### Proteins

The IgGs were all of a quality suitable for in vivo use. Human IL-13 was from PeproTech.

### KinExA Based Measurements at 37° C.

Kinetic Exclusion Assays (KinExA) measurements were performed on a KinExA 3200 (Sapidyne Instruments, Boise, Id., USA) instrument, with the instrument controlled, and the resulting data processed using the supplied KinExA Pro software version 3.2.6.

Receptor ligand mixtures were prepared in sample buffers based on Dulbecco's PBS (D-PBS) supplemented with 1 mg/mL bovine serum albumin (low IgG low endotoxin, Sigma A2058) and 0.02% sodium azide. Flow buffer was the same buffer prepared without the albumin. Due to the long equilibration times at 37° C., all buffers used in the KinExA experiments were 0.2 μm filter sterilised. The fluorescent secondary detection reagent used was the DyLight649-labelled mouse anti-human heavy and light chain specific antibody supplied by Jackson Immunoresearch, (Newmarket, UK). For the sampling bead column, 200 mg of UltraLink Biosupport azlactone beads (Thermo/Pierce 53110) was mixed with 100 μg human IL-13 in 2.5 mL 50 mM sodium hydrogen carbonate pH 8.4 at room temperature with constant agitation for 90 minutes. Rinsing and blocking was achieved with 10 mg/mL BSA in 1 M Tris pH 8.7. Before use, the re-suspended beads were diluted into D-PBS+0.02% sodium azide.

Human IL-13 was titrated into IgG solutions that were fixed at either 100 or 4 pM IgG concentration in order to provide receptor- and K_{D}-controlled dilution series, respectively. These solutions were allowed to come to equilibrium at 37° C. for 12-13 days. KinExA analysis of these equilibrated samples were then performed with the samples and entire KinExA 3200 instrument located in a 37° C. temperature controlled chamber (Series 3 HTCL 750 Temperature Applied Sciences Ltd. Goring-by-sea, West Sussex, BN12 4HF, UK).

During sampling, the KinExA 3200 instrument automatically packed a fresh column of IL-13-conjugated azlactone micro-beads. The pre-equilibrated sample containing antibody, antigen, and Ab/antigen complex was flowed rapidly (0.25 mL/min) through the column to keep the contact time of the sample with the antigen-beads brief. Free antibody bound to the antigen-beads was detected using fluorescent dye labelled Mouse anti Human heavy and light chain specific antibody. By measuring the fraction of free antibody binding sites at a range of different concentrations of IL-13 at a particular fixed concentration of IgG, a KD value was estimated by global least squares (n-curve) fitting, using a 1:1 reversible bimolecular interaction model within the supplied KinExA Pro 3.2.6. software (Sapidyne Instruments, Idaho).

### Results—Kinetic Exclusion Assay (KinExA)

For these affinity measurements, kinetic exclusion assay using KinExA technology (Sapidyne Instruments, Darling and Brault, 2004; ref. 79) was used due to the very high (single digit pM) affinity (K_{D}, dissociation constant) for the interaction of the test IgGs for Human IL-13. Furthermore, a 37° C. based measurement system was used in order 1) to enhance discrimination between the IgG variants and 2) to gain affinity assessments at a more physiologically relevant temperature.

KinExA is a flow spectrofluorometric based methodology that can be used to quantify high affinity interactions, including those in the sub-picomolar range (Rathanaswami et al, 2005; ref. 80). This technology was therefore used to gain a more absolute measure of the affinity of antibody KD values.

Global evaluation of the equilibrated receptor- and KD-controlled dilution series results gave the KD values with calculated 95% confidence intervals as shown in FIG. 7.

### Example 5

### In Vitro Testing of IL-13 Variants

Potency of IL-13 variants (R130, Q130 and Q105) was tested in a TF1 cell proliferation assay. IL-13 variants R130 and Q130 proteins were expressed using Baculovirus/Sf21 system, whereas the Q105 variant was expressed in a CHO system.

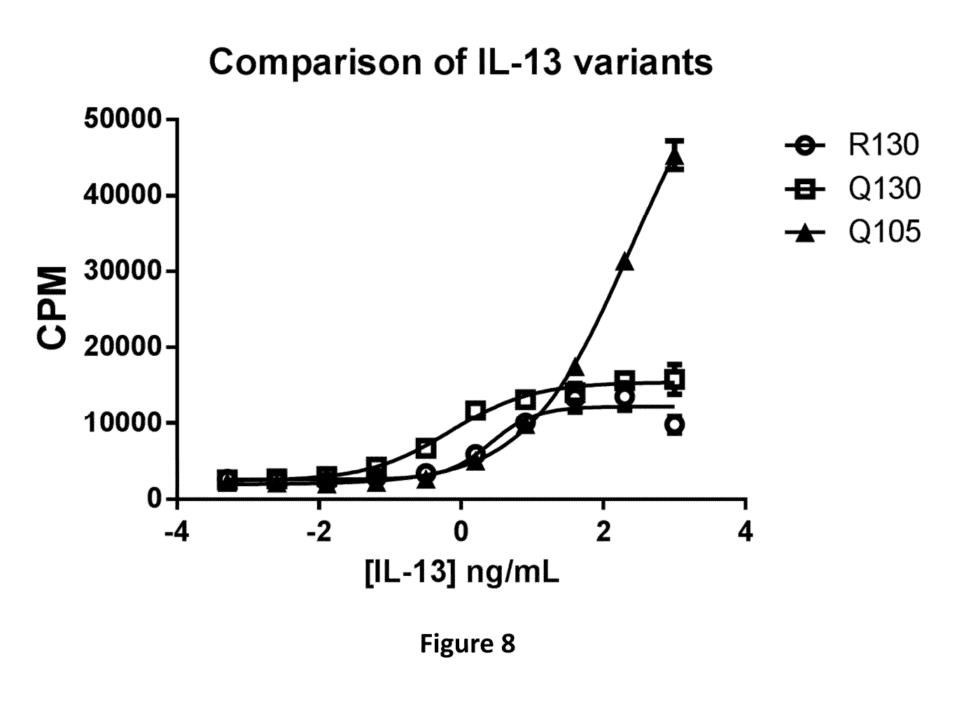

Briefly, TF1 cells (R&D Systems) were washed and re-suspended in assay media to a final concentration of 2×10⁵/mL. One hundred microliters of cells were dispensed into a 96-well flat-bottomed assay plate (Costar). A titration range of the human IL-13 variants was diluted and dispensed into a separate assay plate. All dilutions of cells and IL-13 variants were made in assay media: RPMI-1640 (Gibco), 5% Foetal Bovine Serum, lx Penicillin/Streptomycin (Gibco). One hundred microliters of the IL-13 variant titrations were added to the TF1 cells. Cells with media alone served as negative. Cells were then cultured for 3 days at 37° C., 5% CO₂. After culture period cells were pulsed with 20 microliters/well of [3H]-Thymidine (Perkin-Elmer). Cells were incubated for four hours at 37° C., 5% CO₂. Cells were then harvested on to glass fibre filter plates (Perkin-Elmer) then dry plates for 1 hour at 50° C. 50 microliters/well of Microscint (Perkin-Elmer) was then added, plates sealed and read on a scintillation counter. Results are shown in FIG. 8 and are expressed as counts per minute (C.P.M.).

Data were plotted as C.P.M. versus log(10) concentration of antibody and fitted to a Sigmoidal dose response model (variable slope) Y=Bottom+(Top-Bottom)/(1+10̂((Log EC50−X)*HillSlope)) where; X is the logarithm of concentration. Y is the response; Y starts at Bottom and goes to Top with a sigmoid shape. This is the “four parameter logistic equation. Data analysis was performed using Microsoft Excel and Graphpad Prism software.

### Example 6

### Inhibition of the IL-13 Variant Q105 by 13IL0083

Clone 13NG0083 potency was tested in a TF1 cell proliferation assay. Briefly, TF1 cells (R&D Systems) were washed and re-suspended in assay media to a final concentration of 2×10⁵/mL. One hundred microliters of cells were dispensed into a 96-well flat-bottomed assay plate (Costar). Human interleukin 13 variant Q105 was diluted to a concentration of 40 ng/mL was dispensed into a separate assay plate. A titration range of 13NG0083 or an isotype control was prepared at four times final concentration in a separate assay plate. Equal volumes of the antibody and IL-13 were then mixed and incubated for 30 minutes at room temperature. All dilutions of cells, ligand, and antibodies were made in assay media: Assay media: RPMI-1640 (Gibco), 5% Foetal Bovine Serum, lx Penicillin/Streptomycin (Gibco. One hundred microliters of the antibody/IL-13 combination was then added to the TF1 cells. Cells with media alone or IL-13 alone were used as negative or positive controls respectively. Cells were then cultured for 3 days at 37° C., 5% CO₂. After culture period cells were pulsed with 20 microliters/well of [3H]-Thymidine (Perkin-Elmer). Cells were incubated for four hours at 37° C., 5% CO₂. Cells were then harvested on to glass fibre filter plates (Perkin-Elmer) then dry plates for 1 hour at 50° C. 50 microliters/well of Microscint (Perkin-Elmer) was then added, plates sealed and read on a scintillation counter. Results were expressed as counts per minute (C.P.M.).

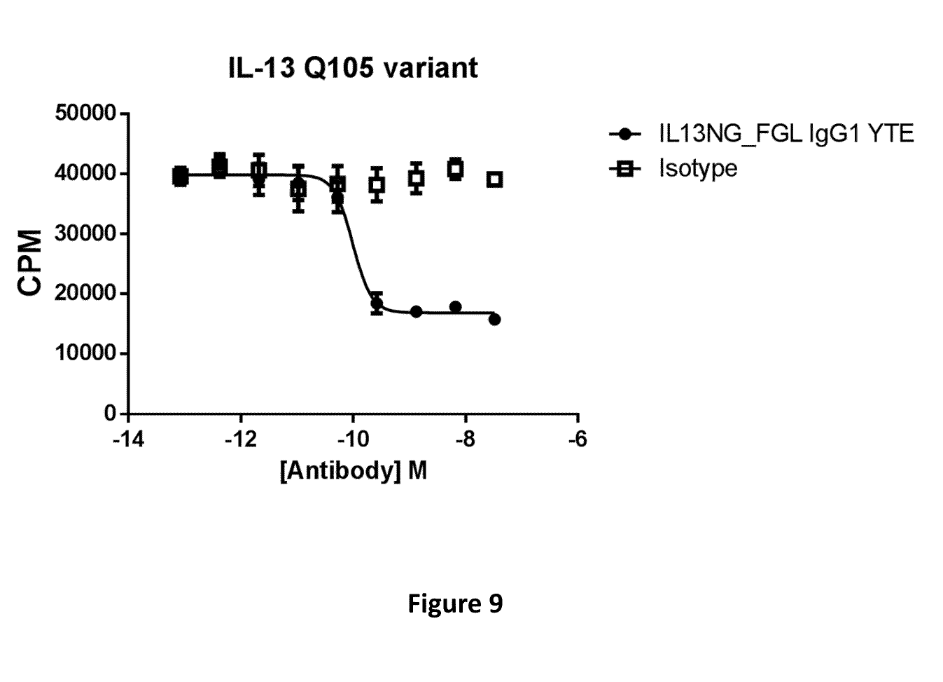

The experiments were performed three times to assess potency of the antibody 13NG0083. As shown in FIG. 9 (a representative single experiment), fully germlined (FGL) 13NG0083 (IgG format with a YTE mutation in the Fc region) inhibited the IL-13 Q105 variant in a TF1 cell proliferation assay. Data was plotted as C.P.M. versus log(10) concentration of antibody and fitted to a Sigmoidal dose response model (variable slope) Y=Bottom+(Top−Bottom)/(1+10̂((Log EC50−X)*HillSlope)) where; X is the logarithm of concentration. Y is the response; Y starts at Bottom and goes to Top with a sigmoid shape. This is the “four parameter logistic equation. Data analysis was performed using Microsoft Excel and Graphpad Prism software.

### Example 7

### IL-13 Human Variants and Cynomolgus IL-13 in a Ligand Receptor Competition Assay

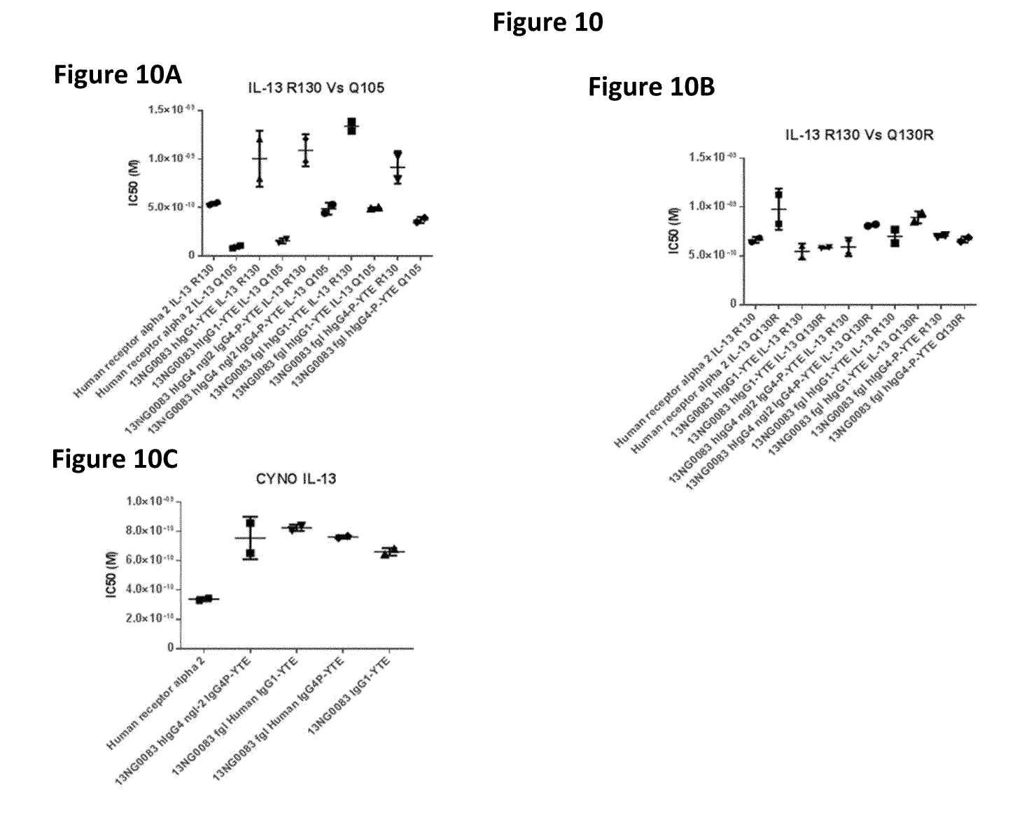

In order to study the ability of 13NG0083 variants to inhibit differing forms of IL-13, the experimental methodology used in the above ligand-receptor assay was repeated, substituting R130 IL-13 for either human Q130R IL-13 (FIG. 10B), human Q105 IL-13 (FIG. 10A), or Cynomolgus IL-13 (FIG. 10C) at the same concentration (312.5 pM). All variants (including 13NG0083 human IgG1+YTE (“hIgG1-YTE”) and 13NG0083 human IgG4-P (IgG4 S241P)+YTE (“IgG4-P-YTE” or “hIgG4-P-YTE”); either fully germlined (“fgl”) or non-germlined (“ngl2”)) were shown to inhibit interactions between IL-13 variants and human IL-13 receptorα2. See FIG. 10. There was little change in potency seen with either the Q105 or Q130R variants of IL-13 when compared to the common variant R130 of IL-13. All clones (including 13NG0083 human IgG1+YTE (“IgG1-YTE”) and 13NG0083 human IgG4-P (IgG4 S241P)+YTE (“IgG4-P-YTE”); either fully germlined (“fgl”) or non-germlined (“ngl2”)) were shown to inhibit the binding to Cynomolgus IL-13 to human IL-13 receptorα2. See FIG. 10.

### Example 8

### Functional Species Cross-Reactivity of 13NG0083 with Mouse and Cynomolgus IL-13

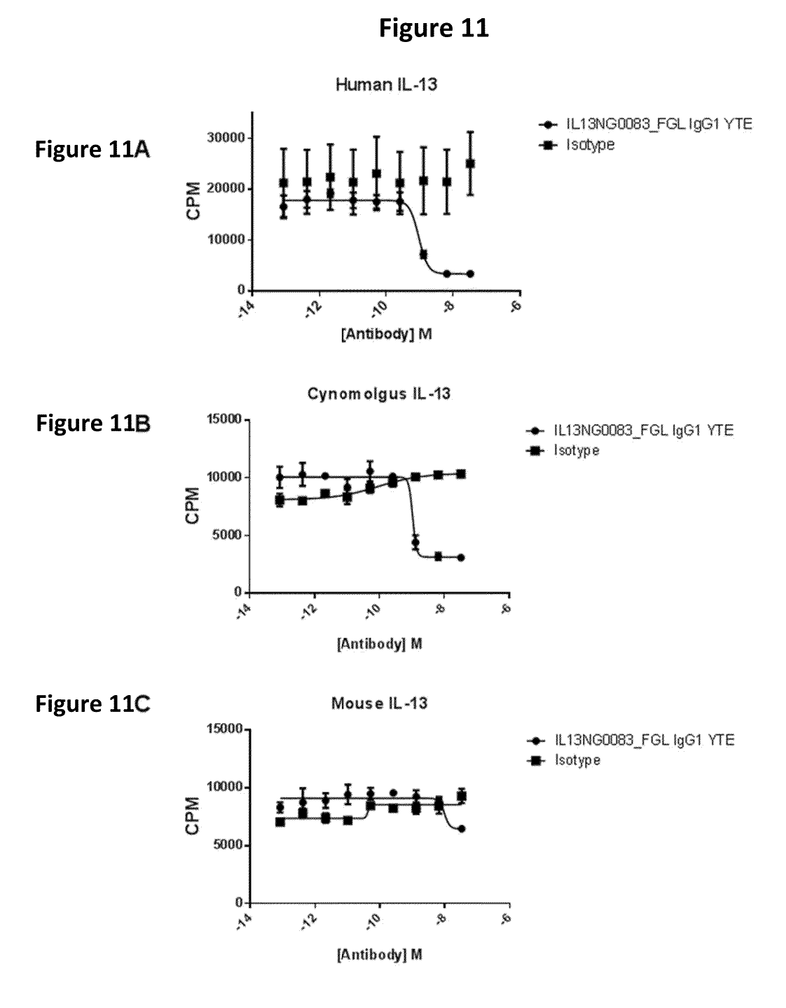

In order to study 13NG0083 species cross reactivity, the experimental methodology described in the TF1 assay in example 2 was repeated, comparing human (A), cynomolgus (B), or mouse IL-13 (C). Results are shown in FIG. 11. Both human and cynomolgus IL-13 was inhibited by IL-13NG0083. Mouse IL-13 supported TF1 proliferation, however no inhibition was observed with IL13NG0083 except a small reduction at the highest concentration of antibody.

### Example 9

### Binding of Human and Cynomolgus FcRn to 13NG0083

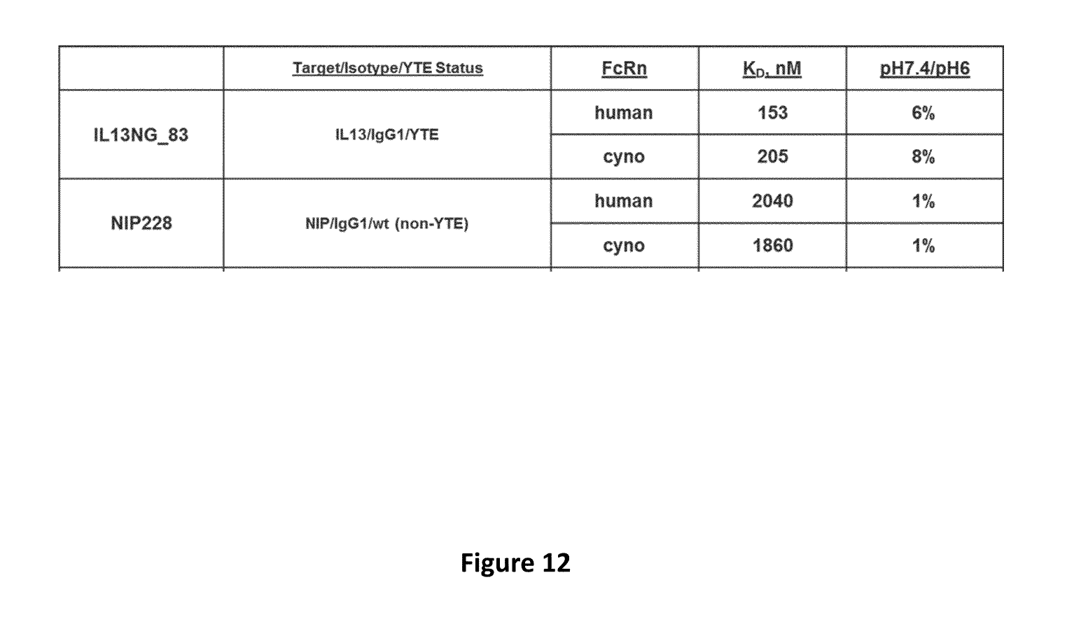

The affinity (KD) for the binding of IL13NG0083 and isotype control IgGs to human FcRn protein (huFcRn) and cynomolgus monkey (cynoFcRn) were measured on a BIAcore 3000 instrument. Briefly, IL13NG0083 and the isotype control IgGs were diluted to a concentration of ˜250 nM (37.5 μg/mL) in 10 mM sodium acetate buffer, pH4, then used to prepare a high density (ranged from ˜2300-˜2600 RU) IgG surfaces on a CM5 sensor chip according to a protocol supplied by the instrument's manufacturer. A reference flow cell surface was also prepared on the sensor chip using the same immobilization protocol, minus the protein. FcRn proteins were produced as described in Dall'Acqua et al, 2002 (ref. no. 81) and Dall'Acqua et al, 2006 (ref. no. 82). Stock solutions of huFcRn and cynoFcRn proteins were prepared at 3000 nM in instrument buffer (50 mM sodium phosphate buffer, pH 6, containing 150 mM NaCl, and 0.05% (v/v) Tween 20 [T20]), then serially diluted (3:1) to 4.11 nM in the same buffer. Each concentration of FcRn was individually injected over the IL13NG0083, isotype control IgG and reference cell surfaces at a flow rate of 5 μL/min, and the binding data was recorded for a period of 50 minutes. Finally, bound FcRn was removed from the sensor chip surfaces by injecting 10 consecutive 60-second pulses of 50 mM sodium phosphate buffer, pH 7.4, containing 150 mM NaCl, and 0.05% (v/v) T20. Several buffer injections were also interspersed throughout the injection series. Later, one of these buffer injections was used along with the reference cell data to correct the raw data sets for injection artifacts (e.g., nonspecific binding) through a technique commonly referred to as “double referencing.” After all binding data was collected, individual data sets were averaged during steady-state binding (Req) at each concentration (C) of FcRn, and then fit to a 1:1 binding model (Req vs. C plot) using the vendor's BIAevaluation software, v. 1.1, to determine the KDs. Results are shown in FIG. 12.

### Example 10

### Stability of 13NG0073 and 13NG0083 in Human Whole Blood

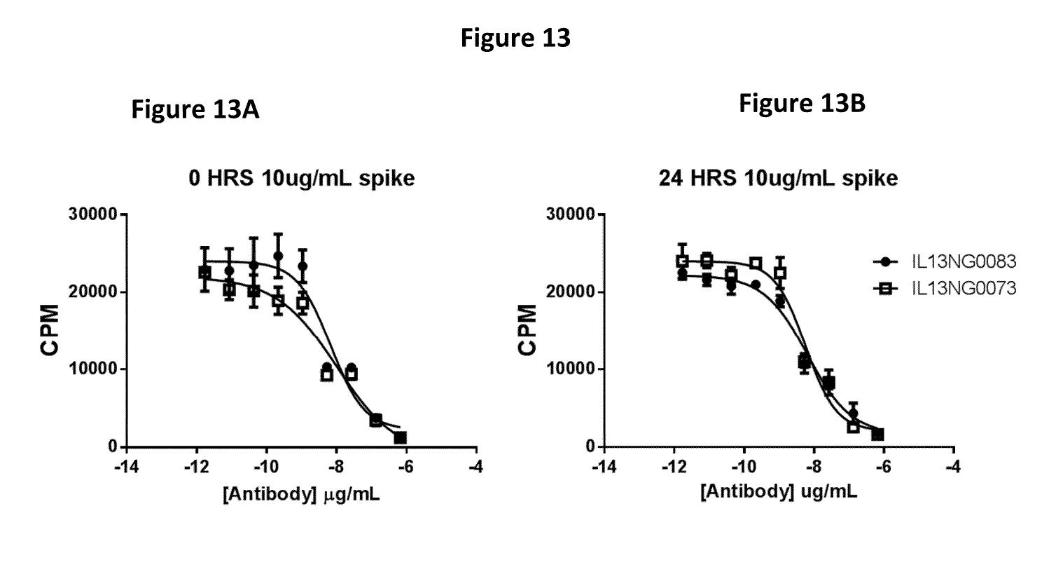

In order to assess the in vivo stability of 13NG0073 and 13NG0083, the antibodies were incubated for either zero or 24 hours in haparinized human blood. Antibody was added to 1 mL of human blood to a final concentration of 10 micrograms per milliliter. After the incubation period, the blood was microfuged to remove cells and the plasma removed. Plasma/antibody was then titrated into a TF1 proliferation assay at an estimated starting concentration of antibodies of 33 nM. The TF1 assay was performed as described previously in Example 2. As shown in FIG. 13, both 13NG0073 and 13NG0083 were stable after incubating in serum for 24 hours as shown by effective inhibition of TF1 cell proliferation.

### Example 11

### IL13NG0083 Expression Engineering

### Materials and Methods

Generation of Reversion Mutants Using Oligo-Directed Mutagenesis

In the first phase of mutagenesis, nine minus strand oligos were designed to mutate the 9 amino acids that constituted the optimised light chain sequence of 13NG0083 back to the unoptimised parental sequence. Kunkel mutagenesis (described previously in Kunkel T A. (1985). Rapid and efficient site-specific mutagenesis without phenotypic selection. “Proceedings of the National Academy of Sciences USA. 82 (2): 488-92 and Sidhu S S and Weiss G A: Constructing Phage Display Libraries by Oligonucleotide-Directed Mutagenesis. Phage Display: A Practical Approach, Edited by Clackson T & Lowman H B 1990, chapter 2: 27-41) was utilised to prepare the individual reversion mutants. Briefly, uracil-containing ssDNA (dU-ssDNA) encoding a VL in a phagemid vector like pEU for example is purified from M13 phage rescued from an E. coli dut-/ung-strain called CJ236. One or several oligonucleotides encoding the desired mutations were annealed to the dU-ssDNA template, extended, and ligated to form covalently closed circular DNA (ccc-DNA). The ccc-DNA transformed E. coli strains such as TG1 and DH5α with high efficiency. The new host destroyed the parental dU-ssDNA strand and synthesized a replacement strand using the mutant strand as a template. Colonies from the transformation were picked into individual wells of a 96-well plate, grown and subjected to PCR followed by sequencing to check for the correct/desired mutation. The resulting dsDNA mutant phagemid was prepared as dsDNA and used for any further purpose.

In the second phase of mutagenesis, further oligos were designed. In some cases up to 2 oligos were used in the same reaction to combine mutations that had given improved expression from the first phase of mutagenesis.

Purification of Plasmids for Expression Evaluation

Separate cultures of the E. coli transformed with the vectors for the light chains and heavy chain were grown overnight and resulting plasmids were purified using a plasmid plus maxi kits (Qiagen). The DNA was then phenol:chloform, chloroform, and then phase lock gel extracted. The DNA was then precipitated within ethanol using sodium acetate to purify salts and proteins away prior to sterile re-suspension with tissue culture grade water in a laminar flow cabinet.

Expression Evaluation in CHO Cells

CHO cells on the day of transformation were seeded at a specific volume and cell density across the required number of 24 deep well plates. DNA was prepared by loading a specific concentration in the presence of Polyethylenimine (PEI) and sodium chloride and distributed across the wells of the 24 well plates after incubation to allow complexing. The plates were then fed with a single volumetric feed at a minimum of 4 hours post transfection. Harvest supernatant was obtained 7 days later and quantified by PrA octet.

### Results

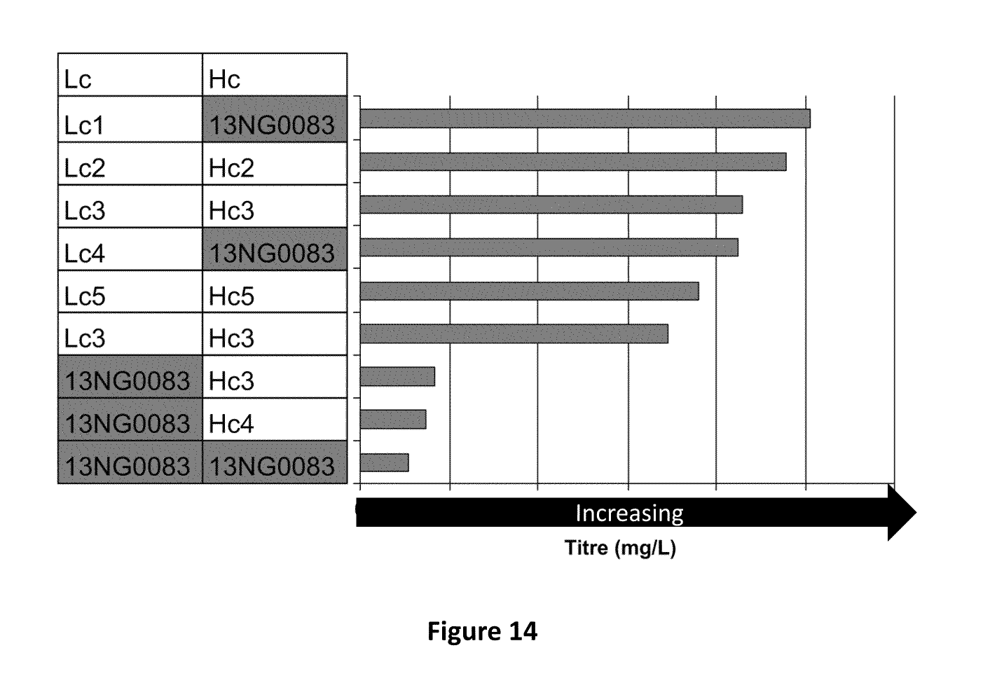

Stable expression of 13NG0083 in CHO cells was observed. However, substituting the 13NG0083 light chain with a number of other light chains consistently resulted in improved expression (titre). See FIG. 14.

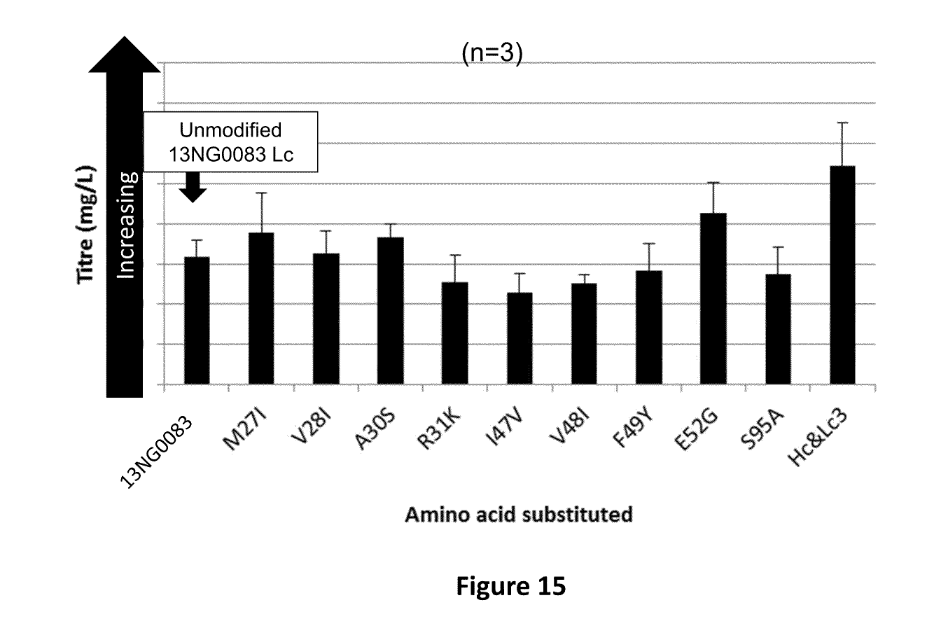

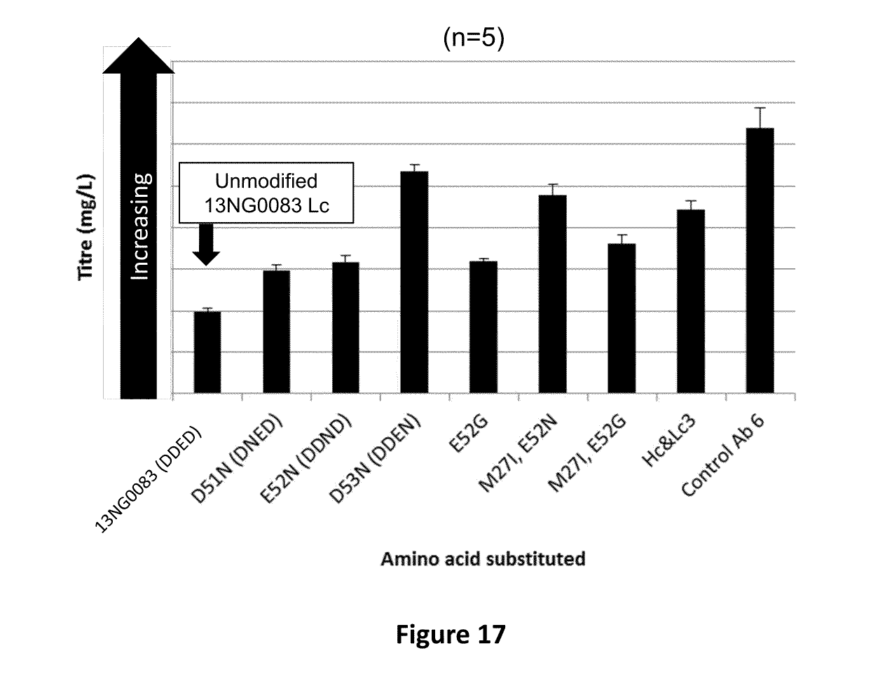

To improve stable expression of 13NG0083, a panel of nine (9) mutants was created using Kunkel mutagenesis to investigate which (if any) of the changes could increase stable expression when co-expressed with the 13NG0083 heavy chain. Two mutants M27I and E52G demonstrated a consistent improvement in stable expression (FIG. 15). When combined, these mutations further improved expression (FIG. 17).

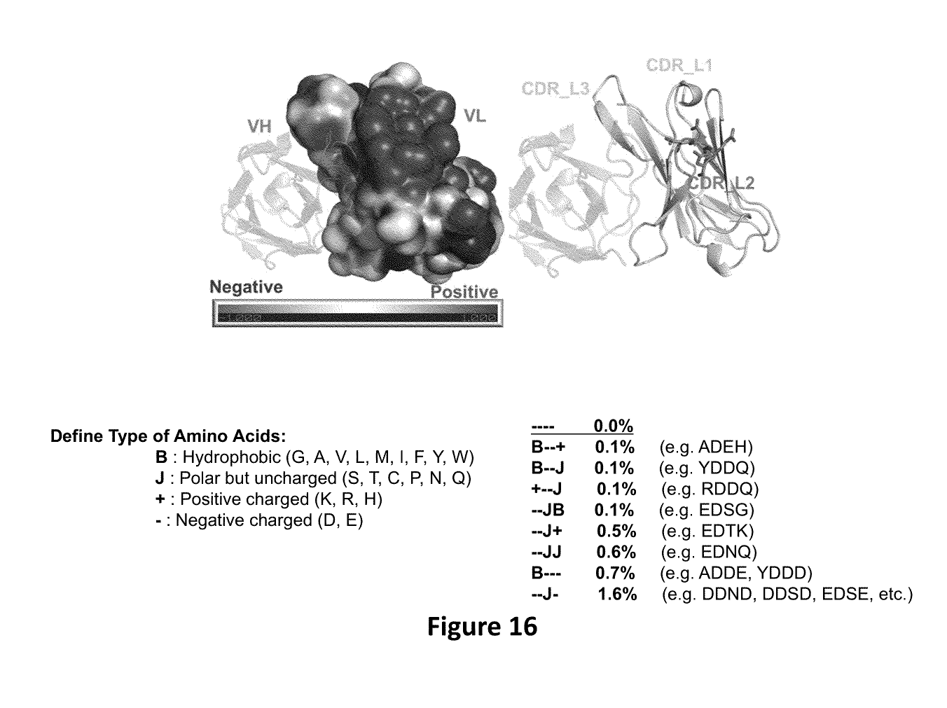

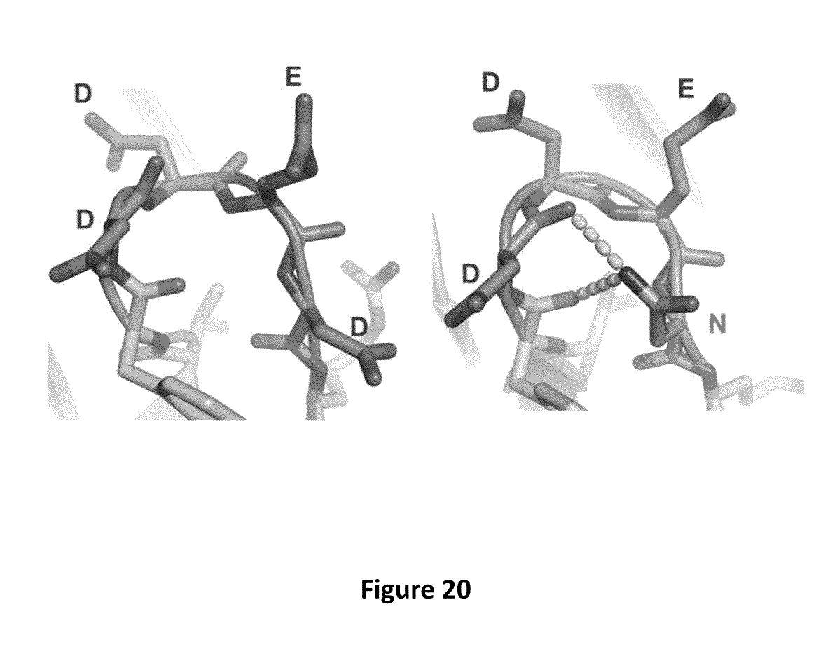

Assessment of the sequence/structure using computational homology modelling and structural bioinformatics, identified three additional mutants for expression profiling to reduce an unusually strong hydrophilic and negative-charged region (50-DDED-53 (SEQ ID NO: 286)) on the tip of VL CDR2 of 13NG0083. Review of ˜1045 antibody structures available in the pdb database (up to 2013) showed that this sequence motif was never observed (FIG. 16). Structural analysis, PDB bioinformatics, and molecular dynamics simulations predicted that removing the negative charge on this loop could increase local structural stability and potentially improve expression. See FIG. 20.

All of the light chain mutants M27I+E52G, M27I+E52N, and the light chain structural mutants (50-) DNED (SEQ ID NO: 287), DDND (SEQ ID NO: 288), or DDEN (SEQ ID NO: 289) (−53) of 13NG0083 resulted in improved expression over unmodified 13NG0083 when co-expressed with 13NG0083 heavy chain. Structural light chain mutant 50-DDEN-53 (SEQ ID NO: 289) showed a marked 270% improvement in expression over 13NG0083 (FIG. 17).

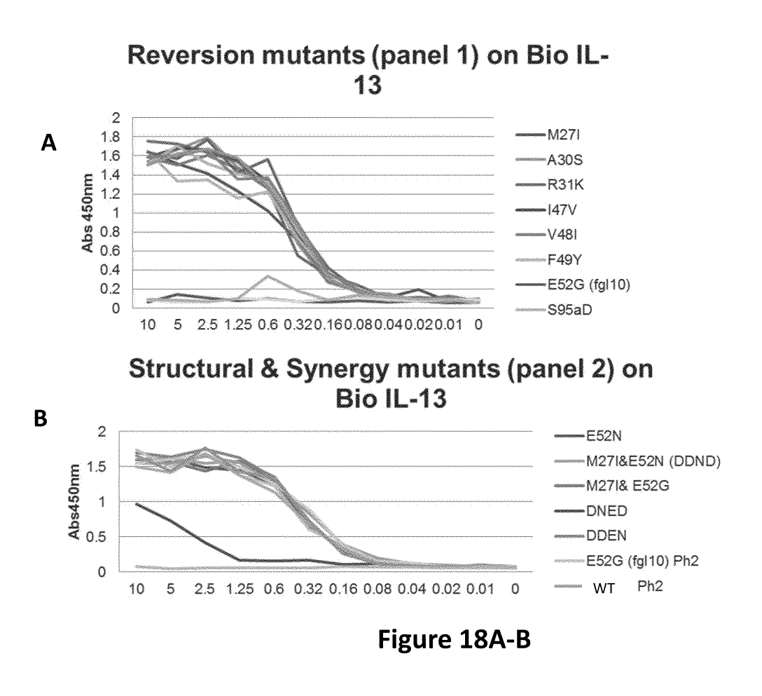

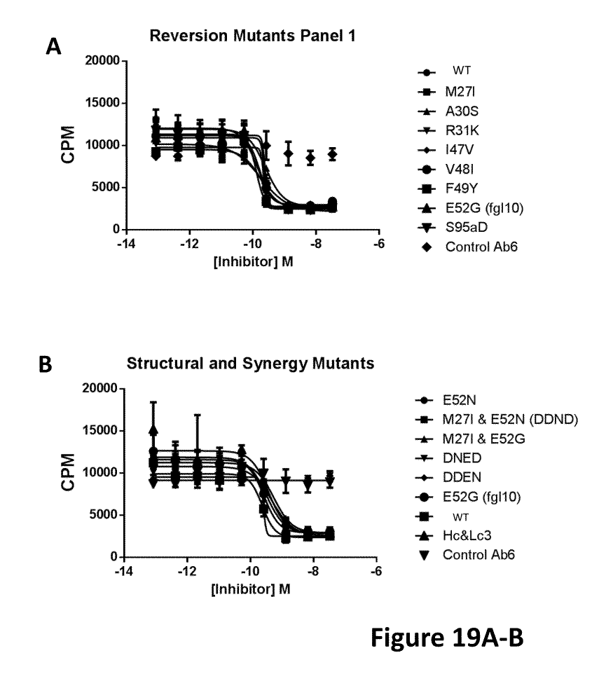

To determine whether the changes in the light chain that resulted in improved expression of 13NG0083 had an impact on binding and potency of 13NG0083 for IL13, a number of biological assays were performed. All of the 13NG0083 light chain mutants tested, except mutant DNED (SEQ ID NO: 287), were observed to bind to IL-13 in an ELISA assay (FIG. 18). In addition, all of the 13NG0083 light chain mutants tested, except mutant DNED (SEQ ID NO: 287), bound and inhibited IL-13-induced proliferation of TF-1 cells with a similar potency as unmodified 13NG0083, including mutant DDEN (SEQ ID NO: 289). (FIG. 19). Accordingly, all of the light chain mutants that resulted in improved expression of 13NG0083 (except mutant DNED (SEQ ID NO: 287)) had no impact on binding and potency of 13NG0083 for IL-13.

TABLE 3
CDR sequences of clones derived from BAK1183H4

| | HCDRs | | | LCDRs | | |
|---|---|---|---|---|---|---|
| Clone | HCDR1 | HCDR2 | HCDR3 | LCDR1 | LCDR2 | LCDR3 |

| | | | | | | |
|---|---|---|---|---|---|---|
| 13NG0083 | NYGLS | WINYDGGN | DSSSSWAR | GGNMVGAR | DDEDRPS | QVWDTGSS |
| | (SEQ ID | TQYGQEFQ | WFFDL | LVH | (SEQ ID | PVV |
| | NO: 13) | G | (SEQ ID | (SEQ ID | NO: 19) | (SEQ ID |
| | | (SEQ ID | NO: 15) | NO: 18) | | NO: 20) |
| | | NO: 14) | | | | |
| 13NG0073 | NYGLS | WINYDGGN | DSSSSWAR | GGNLIGAR | DDIDRPS | QVWDTGSR |
| | (SEQ ID | TQYGQEFQ | WFFDL | LVH | (SEQ ID | PVV (SEQ |
| | NO: 23) | G | (SEQ ID | (SEQ ID | NO: 29) | ID |
| | | (SEQ ID | NO: 25) | NO: 28) | | NO: 30) |
| | | NO: 24) | | | | |
| 13NG0074 | NYGLS | WINYDGGN | DSSSSWAR | GGNLIGAR | DDQDRPS | QVWDTGSL |
| | (SEQ ID | TQYGQEFQ | WFFDL | LVH (SEQ | (SEQ ID | PVV (SEQ |
| | NO: 33) | G | (SEQ ID | ID | NO: 39) | ID |
| | | (SEQ ID | NO: 35) | NO: 38) | | NO: 40) |
| | | NO: 34) | | | | |
| 13NG0068 | NYGLS | WINYDGGN | DSSSSWAR | GGNLIGAR | DDIDRPS | QVWDTGSD |
| | (SEQ ID | TQYGQEFQ | WFFDL | LVH (SEQ | (SEQ ID | PVV (SEQ |
| | NO: 43) | G | (SEQ ID | ID | NO: 49) | ID |
| | | (SEQ ID | NO: 45) | NO: 48) | | NO: 50) |
| | | NO: 44) | | | | |
| 13NG0067 | NYGLS | WINYDGGN | DSSSSWAR | GGNMVGAR | DDIDRPS | QVWDTGSS |
| | (SEQ ID | TQYGQEFQ | WFFDL | LVH (SEQ | (SEQ ID | PVV |
| | NO: 53) | G | (SEQ ID | ID | NO: 59) | (SEQ ID |
| | | (SEQ ID | NO: 55) | NO: 58) | | NO: 60) |
| | | NO: 54) | | | | |
| 13NG0069 | NYGLS | WINYDGGN | DSSSSWAR | GGNMVGAR | DDIDRPS | QVWDTGSR |
| | (SEQ ID | TQYGQEFQ | WFFDL | LVH (SEQ | (SEQ ID | PVV (SEQ |
| | NO: 63) | G | (SEQ ID | ID | NO: 69) | ID |
| | | (SEQ ID | NO: 65) | NO: 68) | | NO: 70) |
| | | NO: 64) | | | | |
| 13NG0076 | NYGLS | WINYDGGN | DSSSSWAR | GGNMVGAR | DDIDRPS | QVWDTGSR |
| | (SEQ ID | TQYGQEFQ | WFFDL | LVH (SEQ | (SEQ ID | PVV |
| | NO: 73) | G | (SEQ ID | ID | NO: 79) | (SEQ ID |
| | | (SEQ ID | NO: 75) | NO: 78) | | NO: 80) |
| | | NO: 74) | | | | |
| 13NG0070 | NYGLS | WINYDGGN | DSSSSWAR | GGNMVGAR | DDEDRPS | QVWDTGSR |
| | (SEQ ID | TQYGQEFQ | WFFDL | LVH (SEQ | (SEQ ID | PVV |
| | NO: 83) | G | (SEQ ID | ID | NO: 89) | (SEQ ID |
| | | (SEQ ID | NO: 85) | NO: 88) | | NO: 90) |
| | | NO: 84) | | | | |
| 13NG0075 | NYGLS | WINYDGGN | DSSSSWAR | GGNMVGAR | DDIDRPS | QVWDTGSR |
| | (SEQ ID | TQYGQEFQ | WFFDL | LVH (SEQ | (SEQ ID | PVV |
| | NO: 93) | G | (SEQ ID | ID | NO: 99) | (SEQ ID |
| | | (SEQ ID | NO: 95) | NO: 98) | | NO: 100) |
| | | NO: 94) | | | | |
| 13NG0077 | NYGLS | WINYDGGN | DSSSSWAR | GGNMVGAY | DDMDRPS | QVWDTGSS |
| | (SEQ ID | TQYGQEFQ | WFFDL | LVH (SEQ | (SEQ ID | PVV |
| | NO: 103) | G | (SEQ ID | ID | NO: 109) | (SEQ ID |
| | | (SEQ ID | NO: 105) | NO: 108) | | NO: 110) |
| | | NO: 104) | | | | |
| 13NG0071 | NYGLS | WINYDGGN | DSSSSWAR | GGNMVGAR | DDEDRPS | QVWDTGSL |
| | (SEQ ID | TQYGQEFQ | WFFDL | LVH (SEQ | (SEQ ID | PVV |
| | NO: 113) | G | (SEQ ID | ID | NO: 119) | (SEQ ID |
| | | (SEQ ID | NO: 115) | NO: 118) | | NO: 120) |
| | | NO: 114) | | | | |
| 13NG0072 | NYGLS | WINYDGGN | DSSSSWAR | GGNMVGAR | DDMDRPS | QVWDTGSR |
| | (SEQ ID | TQYGQEFQ | WFFDL | LVH (SEQ | (SEQ ID | PVV (SEQ |
| | NO: 123) | G | (SEQ ID | ID | NO: 129) | ID |
| | | (SEQ ID | NO: 125) | NO: 128) | | NO: 130) |
| | | NO: 124) | | | | |
| 13NG0024 | NYGLS | WINYDGGN | DSSSSWAR | GGNLLGAR | DDGDRPS | QVWDTGSD |
| | (SEQ ID | TQYGQEFQ | WFFDL | LVH (SEQ | (SEQ ID | PVV |
| | NO: 13) | G | (SEQ ID | ID | NO: 249) | (SEQ ID |
| | | (SEQ ID | NO: 15) | NO: 275) | | NO: 250) |
| | | NO: 14) | | | | |
| 13NG0033 | NYGLS | WINYDGGN | DSSSSWAR | GGNLIGAR | DDGDRPS | QVWDTGSS |
| | (SEQ ID | TQYGQEFQ | WFFDL | LVH (SEQ | (SEQ ID | PVV |
| | NO: 13) | G | (SEQ ID | ID NO: 28) | NO: 249) | (SEQ ID |
| | | (SEQ ID | NO: 15) | | | NO: 160) |
| | | NO: 14) | | | | |
| 13NG0025 | NYGLS | WINYDGGN | DSSSSWAR | GGNMVGAR | DDGDRPS | QVWDTGSD |
| | (SEQ ID | TQYGQEFQ | WFFDL | LVH (SEQ | (SEQ ID | PVV (SEQ |
| | NO: 243) | G (SEQ | (SEQ ID | ID | NO: 249) | ID |
| | | ID | NO: 245) | NO: 248) | | NO: 250) |
| | | NO: 244) | | | | |
| 13NG0088 | NYGLS | WINYDGGN | DSSSSWAR | GGNLIGAR | DDIDRPS | QVWDTGSR |
| | (SEQ ID | TQYGQEFQ | WFFDL | LVH | (SEQ ID | PVV (SEQ |
| | NO: 173) | G (SEQ | (SEQ ID | (SEQ ID | NO: 179) | ID |
| | | ID | NO: 175) | NO: 178) | | NO: 180) |
| | | NO: 174) | | | | |
| 13NG0081 | NYGLS | WINYDGGN | DSSSSWAR | GGNLIGAR | DDMDRPS | QVWDTGSR |
| | (SEQ ID | TQYGQEFQ | WFFDL | LVH (SEQ | (SEQ ID | PVV (SEQ |
| | NO: 133) | G (SEQ | (SEQ ID | ID | NO: 139) | ID |
| | | ID | NO: 135) | NO: 138) | | NO: 140) |
| | | NO: 134) | | | | |
| 13NG0079 | NYGLS | WINYDGGN | DSSSSWAR | GGNLIGAR | DDMDRPS | QVWDTGSL |
| | (SEQ ID | TQYGQEFQ | WFFDL | LVH (SEQ | (SEQ ID | PVV (SEQ |
| | NO: 143) | G (SEQ | (SEQ ID | ID | NO: 149) | ID |
| | | ID | NO: 145) | NO: 148) | | NO: 150) |
| | | NO: 144) | | | | |
| 13NG0086 | NYGLS | WINYDGGN | DSSSSWAR | GGNMVGAR | DDMDRPS | QVWDTGSR |
| | (SEQ ID | TQYGQEFQ | WFFDL | LVH (SEQ | (SEQ ID | PVV (SEQ |
| | NO: 163) | G (SEQ | (SEQ ID | ID | NO: 169) | ID |
| | | ID | NO: 165) | NO: 168) | | NO: 170) |
| | | NO: 164) | | | | |
| 13NG0085 | NYGLS | WINYDGGN | DSSSSWAR | GGNMVGAR | DDIDRPS | QVWDTGSR |
| | (SEQ ID | TQYGQEFQ | WFFDL | LVH (SEQ | (SEQ ID | PVV (SEQ |
| | NO: 213) | G (SEQ | (SEQ ID | ID | NO: 219) | ID |
| | | ID | NO: 215) | NO: 218) | | NO: 220) |
| | | NO: 214) | | | | |
| 13NG0082 | NYGLS | WINYDGGN | DSSSSWAR | GGNMVGAR | DDEDRPS | QVWDTGSR |
| | (SEQ ID | TQYGQEFQ | WFFDL | LVH (SEQ | (SEQ ID | PVV (SEQ |
| | NO: 193) | G (SEQ | (SEQ ID | ID | NO: 199) | ID |
| | | ID | NO: 195) | NO: 198) | | NO: 200) |
| | | NO: 194) | | | | |
| 13NG0084 | NYGLS | WINYDGGN | DSSSSWAR | GGNLIAAR | DDEDRPS | QVWDTGSS |
| | (SEQ ID | TQYGQEFQ | WFFDL | LVH (SEQ | (SEQ ID | PVV (SEQ |
| | NO: 183) | G (SEQ | (SEQ ID | ID | NO: 189) | ID |
| | | ID | NO: 185) | NO: 188) | | NO: 190) |
| | | NO: 184) | | | | |
| 13NG0087 | NYGLS | WINYDGGN | DSSSSWAR | GGNMVAAR | DDQDRPS | QVWDTGSL |
| | (SEQ ID | TQYGQEFQ | WFFDL | LVH (SEQ | (SEQ ID | PVV (SEQ |
| | NO: 203) | G (SEQ | (SEQ ID | ID | NO: 209) | ID |
| | | ID | NO: 205) | NO: 208) | | NO: 210) |
| | | NO: 204) | | | | |
| 13NG0080 | NYGLS | WINYDGGN | DSSSSWAR | GGNLIAAR | DDEDRPS | QVWDTGSS |
| | (SEQ ID | TQYGQEFQ | WFFDL | LVH (SEQ | (SEQ ID | PVV (SEQ |
| | NO: 153) | G (SEQ | (SEQ ID | ID | NO: 159) | ID |
| | | ID | NO: 155) | NO: 158) | | NO: 160) |
| | | NO: 154) | | | | |
| 13NG0078 | NYGLS | WINYDGGN | DSSSSWAR | GGNLIAAR | DDQDRPS | QVWDTGSL |
| | (SEQ ID | TQYGQEFQ | WFFDL | LVH (SEQ | (SEQ ID | P (SEQ |
| | NO: 223) | G (SEQ | (SEQ ID | ID | NO: 229) | ID |
| | | ID | NO: 225) | NO: 228) | | NO: 230) |
| | | NO: 224) | | | | |

TABLE 4
CDR sequences of clones derived from BAK1183H4

| | HCDRs | | | LCDRs | | |
|---|---|---|---|---|---|---|
| Clone | HCDR1 | HCDR2 | HCDR3 | LCDR1 | LCDR2 | LCDR3 |
| 13NG0083 | NYGLS | WINYDGGN | DSSSSWAR | GGNMVGAR | DDEDRPS | QVWDTGSS |
| | (SEQ ID | TQYGQEFQ | WFFDL | LVH | (SEQ ID | PVV |
| | NO: 13) | G | (SEQ ID | (SEQ ID | NO: 19) | (SEQ ID |
| | | (SEQ ID | NO: 15) | NO: 18) | | NO: 20) |
| | | NO: 14) | | | | |
| 13NG0073 | NYGLS | WINYDGGN | DSSSSWAR | GGNLIGAR | DDIDRPS | QVWDTGSR |
| | (SEQ ID | TQYGQEFQ | WFFDL | LVH | (SEQ ID | PVV (SEQ |
| | NO: 23) | G | (SEQ ID | (SEQ ID | NO: 29) | ID |
| | | (SEQ ID | NO: 25) | NO: 28) | | NO: 30) |
| | | NO: 24) | | | | |
| 13NG0074 | NYGLS | WINYDGGN | DSSSSWAR | GGNLIGAR | DDQDRPS | QVWDTGSL |
| | (SEQ ID | TQYGQEFQ | WFFDL | LVH (SEQ | (SEQ ID | PVV (SEQ |
| | NO: 33) | G | (SEQ ID | ID | NO: 39) | ID |
| | | (SEQ ID | NO: 35) | NO: 38) | | NO: 40) |
| | | NO: 34) | | | | |
| 13NG0068 | NYGLS | WINYDGGN | DSSSSWAR | GGNLIGAR | DDIDRPS | QVWDTGSD |
| | (SEQ ID | TQYGQEFQ | WFFDL | LVH (SEQ | (SEQ ID | PVV (SEQ |
| | NO: 43) | G | (SEQ ID | ID | NO: 49) | ID |
| | | (SEQ ID | NO: 45) | NO: 48) | | NO: 50) |
| | | NO: 44) | | | | |
| 13NG0067 | NYGLS | WINYDGGN | DSSSSWAR | GGNMVGAR | DDIDRPS | QVWDTGSS |
| | (SEQ ID | TQYGQEFQ | WFFDL | LVH (SEQ | (SEQ ID | PVV |
| | NO: 53) | G | (SEQ ID | ID | NO: 59) | (SEQ ID |
| | | (SEQ ID | NO: 55) | NO: 58) | | NO: 60) |
| | | NO: 54) | | | | |
| 13NG0069 | NYGLS | WINYDGGN | DSSSSWAR | GGNMVGAR | DDIDRPS | QVWDTGSR |
| | (SEQ ID | TQYGQEFQ | WFFDL | LVH (SEQ | (SEQ ID | PVV (SEQ |
| | NO: 63) | G | (SEQ ID | ID | NO: 69) | ID |
| | | (SEQ ID | NO: 65) | NO: 68) | | NO: 70) |
| | | NO: 64) | | | | |
| 13NG0076 | NYGLS | WINYDGGN | DSSSSWAR | GGNMVGAR | DDIDRPS | QVWDTGSR |
| | (SEQ ID | TQYGQEFQ | WFFDL | LVH (SEQ | (SEQ ID | PVV |
| | NO: 73) | G | (SEQ ID | ID | NO: 79) | (SEQ ID |
| | | (SEQ ID | NO: 75) | NO: 78) | | NO: 80) |
| | | NO: 74) | | | | |
| 13NG0070 | NYGLS | WINYDGGN | DSSSSWAR | GGNMVGAR | DDEDRPS | QVWDTGSR |
| | (SEQ ID | TQYGQEFQ | WFFDL | LVH (SEQ | (SEQ ID | PVV |
| | NO: 83) | G | (SEQ ID | ID | NO: 89) | (SEQ ID |
| | | (SEQ ID | NO: 85) | NO: 88) | | NO: 90) |
| | | NO: 84) | | | | |
| 13NG0075 | NYGLS | WINYDGGN | DSSSSWAR | GGNMVGAR | DDIDRPS | QVWDTGSR |
| | (SEQ ID | TQYGQEFQ | WFFDL | LVH (SEQ | (SEQ ID | PVV |
| | NO: 93) | G | (SEQ ID | ID | NO: 99) | (SEQ ID |
| | | (SEQ ID | NO: 95) | NO: 98) | | NO: 100) |
| | | NO: 94) | | | | |
| 13NG0077 | NYGLS | WINYDGGN | DSSSSWAR | GGNMVGAY | DDMDRPS | QVWDTGSS |
| | (SEQ ID | TQYGQEFQ | WFFDL | LVH (SEQ | (SEQ ID | PVV |
| | NO: 103) | G | (SEQ ID | ID | NO: 109) | (SEQ ID |
| | | (SEQ ID | NO: 105) | NO: 108) | | NO: 110) |
| | | NO: 104) | | | | |
| 13NG0071 | NYGLS | WINYDGGN | DSSSSWAR | GGNMVGAR | DDEDRPS | QVWDTGSL |
| | (SEQ ID | TQYGQEFQ | WFFDL | LVH (SEQ | (SEQ ID | PVV |
| | NO: 113) | G | (SEQ ID | ID | NO: 119) | (SEQ ID |
| | | (SEQ ID | NO: 115) | NO: 118) | | NO: 120) |
| | | NO: 114) | | | | |
| 13NG0072 | NYGLS | WINYDGGN | DSSSSWAR | GGNMVGAR | DDMDRPS | QVWDTGSR |
| | (SEQ ID | TQYGQEFQ | WFFDL | LVH (SEQ | (SEQ ID | PVV (SEQ |
| | NO: 123) | G | (SEQ ID | ID | NO: 129) | ID |
| | | (SEQ ID | NO: 125) | NO: 128) | | NO: 130) |
| | | NO: 124) | | | | |
| 13NG0024 | NYGLS | WINYDGGN | DSSSSWAR | GGNLLGAR | DDGDRPS | QVWDTGSD |
| | (SEQ ID | TQYGQEFQ | WFFDL | LVH (SEQ | (SEQ ID | PVV |
| | NO: 13) | G | (SEQ ID | ID NO: | NO: 249) | (SEQ ID |
| | | (SEQ ID | NO: 15) | 275) | | NO: 250) |
| | | NO: 14) | | | | |
| 13NG0033 | NYGLS | WINYDGGN | DSSSSWAR | GGNLIGAR | DDGDRPS | QVWDTGSS |
| | (SEQ ID | TQYGQEFQ | WFFDL | LVH (SEQ | (SEQ ID | PVV |
| | NO: 13) | G | (SEQ ID | ID NO: | NO: 249) | (SEQ ID |
| | | (SEQ ID | NO: 15) | 28) | | NO: 160) |
| | | NO: 14) | | | | |

TABLE 5
CDR sequences of selected clones derived from BAK1183H4

| | HCDRs | | | LCDRs | | |
|---|---|---|---|---|---|---|
| Clone | HCDR1 | HCDR2 | HCDR3 | LCDR1 | LCDR2 | LCDR3 |
| 13NG0083 | NYGLS | WINYDGGN | DSSSSWAR | GGNMVGAR | DDEDRPS | QVWDTGSS |
| | (SEQ ID | TQYGQEFQ | WFFDL | LVH | (SEQ ID | PVV |
| | NO: 13) | G | (SEQ ID | (SEQ ID | NO: 19) | (SEQ ID |
| | | (SEQ ID | NO: 15) | NO: 18) | | NO: 20) |
| | | NO: 14) | | | | |
| 13NG0073 | NYGLS | WINYDGGN | DSSSSWAR | GGNLIGAR | DDIDRPS | QVWDTGSR |
| | (SEQ ID | TQYGQEFQ | WFFDL | LVH | (SEQ ID | PVV (SEQ |
| | NO: 23) | G | (SEQ ID | (SEQ ID | NO: 29) | ID |
| | | (SEQ ID | NO: 25) | NO: 28) | | NO: 30) |
| | | NO: 24) | | | | |
| 13NG0074 | NYGLS | WINYDGGN | DSSSSWAR | GGNLIGAR | DDQDRPS | QVWDTGSL |
| | (SEQ ID | TQYGQEFQ | WFFDL | LVH (SEQ | (SEQ ID | PVV (SEQ |
| | NO: 33) | G | (SEQ ID | ID | NO: 39) | ID |
| | | (SEQ ID | NO: 35) | NO: 38) | | NO: 40) |
| | | NO: 34) | | | | |
| 13NG0024 | NYGLS | WINYDGGN | DSSSSWAR | GGNLLGAR | DDGDRPS | QVWDTGSD |
| | (SEQ ID | TQYGQEFQ | WFFDL | LVH (SEQ | (SEQ ID | PVV |
| | NO: 13) | G | (SEQ ID | ID NO: | NO: 249) | (SEQ ID |
| | | (SEQ ID | NO: 15) | 275) | | NO: 250) |
| | | NO: 14) | | | | |
| 13NG0033 | NYGLS | WINYDGGN | DSSSSWAR | GGNLIGAR | DDGDRPS | QVWDTGSS |
| | (SEQ ID | TQYGQEFQ | WFFDL | LVH (SEQ | (SEQ ID | PVV |
| | NO: 13) | G | (SEQ ID | ID NO: | NO: 249) | (SEQ ID |
| | | (SEQ ID | NO: 15) | 28) | | NO: 160) |
| | | NO: 14) | | | | |
| 13NG0071 | NYGLS | WINYDGGN | DSSSSWAR | GGNMVGAR | DDEDRPS | QVWDTGSL |
| | (SEQ ID | TQYGQEFQ | WFFDL | LVH (SEQ | (SEQ ID | PVV |
| | NO: 113) | G | (SEQ ID | ID | NO: 119) | (SEQ ID |
| | | (SEQ ID | NO: 115) | NO: 118) | | NO: 120) |
| | | NO: 114) | | | | |

TABLE 6
CDR sequences of selected clones derived from BAK1183H4

| | HCDRs | | | LCDRs | | |
|---|---|---|---|---|---|---|
| Clone | HCDR1 | HCDR2 | HCDR3 | LCDR1 | LCDR2 | LCDR3 |
| 13NG0083 | NYGLS | WINYDGGN | DSSSSWAR | GGNMVGAR | DDEDRPS | QVWDTGSS |
| | (SEQ ID | TQYGQEFQ | WFFDL | LVH | (SEQ ID | PVV |
| | NO: 13) | G | (SEQ ID | (SEQ ID | NO: 19) | (SEQ ID |
| | | (SEQ ID | NO: 15) | NO: 18) | | NO: 20) |
| | | NO: 14) | | | | |
| 13NG0073 | NYGLS | WINYDGGN | DSSSSWAR | GGNLIGAR | DDIDRPS | QVWDTGSR |
| | (SEQ ID | TQYGQEFQ | WFFDL | LVH | (SEQ ID | PVV (SEQ |
| | NO: 23) | G | (SEQ ID | (SEQ ID | NO: 29) | ID |
| | | (SEQ ID | NO: 25) | NO: 28) | | NO: 30) |
| | | NO: 24) | | | | |
| 13NG0074 | NYGLS | WINYDGGN | DSSSSWAR | GGNLIGAR | DDQDRPS | QVWDTGSL |
| | (SEQ ID | TQYGQEFQ | WFFDL | LVH (SEQ | (SEQ ID | PVV (SEQ |
| | NO: 33) | G | (SEQ ID | ID | NO: 39) | ID |
| | | (SEQ ID | NO: 35) | NO: 38) | | NO: 40) |
| | | NO: 34) | | | | |

The foregoing description of the specific embodiments will so fully reveal the general nature of the disclosure that others can, by applying knowledge within the skill of the art, readily modify and/or adapt for various applications such specific embodiments, without undue experimentation, without departing from the general concept of the present disclosure. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The breadth and scope of the present disclosure should not be limited by any of the above-described exemplary embodiments, but should be defined only in accordance with the following claims and their equivalents.

All publications, patents, patent applications, and/or other documents cited in this application are incorporated by reference in their entirety for all purposes to the same extent as if each individual publication, patent, patent application, and/or other document were individually indicated to be incorporated by reference for all purposes.

### REFERENCES

1. McKenzie, A. N., et al. J Immunol, 1993. 150(12): p. 5436-44.
2. Minty, A., et al. Nature, 1993. 362(6417): p. 248-50.
3. Nakamura, Y., et al. Am J Respir Cell Mol Biol, 1996. 15(5): p. 680-7.
4. Robinson, D. S., et al. N Engl J Med, 1992. 326(5): p. 298-304.
5. Walker, C., et al. Am J Respir Crit Care Med, 1994. 150(4): p. 1038-48.
6. Humbert, M., et al. Am J Respir Crit Care Med, 1996. 154(5): p. 1497-504.
7. Corrigan, C. J. and A. B. Kay Int Arch Allergy Appl Immunol, 1991. 94(1-4): p. 270-1.
8. Bentley, A. M., et al. Am J Respir Cell Mol Biol, 1993. 8(1): p. 35-42.
9. Murata, T., et al. Int J Hematol, 1999. 69(1): p. 13-20.
10. Andrews, A. L., et al. J Biol Chem, 2002. 277(48): p. 46073-8.
11. Miloux, B., et al. FEBS Lett, 1997. 401(2-3): p. 163-6.
12. Hilton, D. J., et al. Proc Natl Acad Sci USA, 1996. 93(1): p. 497-501.
13. Kuperman, D., et al. J Exp Med, 1998. 187(6): p. 939-48.
14. Nelms, K., et al. Annu Rev Immunol, 1999. 17: p. 701-38.
15. Zhang, J. G., et al. J Biol Chem, 1997. 272(14): p. 9474-80.
16. Caput, D., et al. J Biol Chem, 1996. 271(28): p. 16921-6.
17. Kawakami, K., et al. Blood, 2001. 97(9): p. 2673-9.
18. Wood, N., et al. J Exp Med, 2003. 197(6): p. 703-709.
19. Chiaramonte, M. G., et al. J Exp Med, 2003. 197(6): p. 687-701.
20. Beasley, R., et al. J Allergy Clin Immunol, 2000. 105(2 Pt 2): p. S466-72.
21. Peat, J. K. and J. Li J Allergy Clin Immunol, 1999. 103(1 Pt 1): p. 1-10.
22. Society, B. T., British guideline on the management of asthma. Thorax, 2003. 58 Suppl 1: p. i1-94.
23. GINA, Global Strategy for Asthma Management and Prevention. 2002, National Insitute of Health.
24. Milgrom, H., B. Bender, and F. Wamboldt. Ann Allergy Asthma Immunol, 2002. 88(5): p. 429-31.
25. Fish, L. and C. L. Lung, Adherence to asthma therapy. Ann Allergy Asthma Immunol, 2001. 86(6 Suppl 1): p. 24-30.
26. Bender, B. G. J Allergy Clin Immunol, 2002. 109(6 Suppl): p. S554-9.
27. Wills-Karp, M., et al. Science, 1998. 282(5397): p. 2258-61.
28. Grunig, G., et al. Science, 1998. 282(5397): p. 2261-3.
29. Venkayya, R., et al. Am J Respir Cell Mol Biol, 2002. 26(2): p. 202-8.
30. Morse, B., et al. Am J Physiol Lung Cell Mol Physiol, 2002. 282(1): p. L44-9.
31. Zhu, Z., et al. J Clin Invest, 1999. 103(6): p. 779-88.
32. Walter, D. M., et al. J Immunol, 2001. 167(8): p. 4668-75.
33. Cohn, L., J. S. Tepper, and K. Bottomly. J Immunol, 1998. 161(8): p. 3813-6.
34. Taube, C., et al. J Immunol, 2002. 169(11): p. 6482-9.
35. Yang, E. S., et al. J. Allergy Immunol., 2002. 109: p. A168.
36. Blease, K., et al. J Immunol, 2001. 166(8): p. 5219-24.
37. Heinzmann, A., et al. Hum Mol Genet, 2000. 9(4): p. 549-59.
38. Howard, T. D., et al. Am J Hum Genet, 2002. 70(1): p. 230-6.
39. Kauppi, P., et al. Genomics, 2001. 77(1-2): p. 35-42.
40. Graves, P. E., et al. J Allergy Clin Immunol, 2000. 105(3): p. 506-13.
41. Arima, K., et al. J Allergy Clin Immunol, 2002. 109(6): p. 980-7.
42. van der Pouw Kraan, T. C., et al. Genes Immun, 1999. 1(1): p. 61-5.
43. Humbert, M., et al. J Allergy Clin Immunol, 1997. 99(5): p. 657-65.
44. Kotsimbos, T. C., P. Ernst, and Q. A. Hamid, Proc Assoc Am Physicians, 1996. 108(5): p. 368-73.
45. Komai-Koma, M., F. Y. Liew, and P. C. Wilkinson, J Immunol, 1995. 155(3): p. 1110-6.
46. Naseer, T., et al. Am J Respir Crit Care Med, 1997. 155(3): p. 845-51.
47. Huang, S. K., et al. J Immunol, 1995. 155(5): p. 2688-94.
48. Kroegel, C., et al. Eur Respir J, 1996. 9(5): p. 899-904.
49. Ohshima, Y., et al. Pediatr Res, 2002. 51(2): p. 195-200.
50. Hasegawa, M., et al. J Rheumatol, 1997. 24(2): p. 328-32.
51. Hancock, A., et al. Am J Respir Cell Mol Biol, 1998. 18(1): p. 60-5.
52. Lee, C. G., et al. J Exp Med, 2001. 194(6): p. 809-21.
53. Jain-Vora, S., et al. Am J Respir Cell Mol Biol, 1997. 17(5): p. 541-51.
54. Fallon, P. G., et al. J Immunol, 2000. 164(5): p. 2585-91.
55. Chiaramonte, M. G., et al. J Clin Invest, 1999. 104(6): p. 777-85.
56. Chiaramonte, M. G., et al. Hepatology, 2001. 34(2): p. 273-82.
57. Sluiter, H. J., et al. Eur Respir J, 1991. 4(4): p. 479-89.
58. Zheng, T., et al. J Clin Invest, 2000. 106(9): p. 1081-93.
59. Tashkin, D. P., et al., Methacholine reactivity predicts changes in lung function over time in smokers with early chronic obstructive pulmonary disease. The Lung Health Study Research Group. Am J Respir Crit Care Med, 1996. 153(6 Pt 1): p. 1802-11.
60. Van Der Pouw Kraan, T. C., et al. Genes Immun, 2002. 3(7): p. 436-9.
61. Skinnider, B. F., et al. Blood, 2001. 97(1): p. 250-5.
62. Kapp, U., et al. J Exp Med, 1999. 189(12): p. 1939-46.
63. Fiumara, P., F. Cabanillas, and A. Younes, Blood, 2001. 98(9): p. 2877-8.
64. Terabe, M., et al. Nat Immunol, 2000. 1(6): p. 515-20.
65. Ahlers, J. D., et al. Proc Natl Acad Sci USA, 2002. 99(20): p. 13020-5.
66. Belvisi, M. G., et al., Pulm Pharmacol Ther, 2001. 14(3): p. 221-7.
67. Barnes, P. J., et al. Eur Respir J, 1996. 9(4): p. 636-42.
68. Barnes, P. J., Pharmacol Ther, 2003. 97(1): p. 87-94.
69. Wardlaw, A. J., Clin Med, 2001. 1(3): p. 214-8.
70. Kabat E A et al (1991): Sequences of Proteins of Immunological Interest, 5th Edition. US Department of Health and Human Services, Public Service, NIH, Washington.
71. Kontermann R and Dubel Stefan; (2001) Antibody Engineering, Springer Laboratory Manuals.
72. Csonka E et al (2000) Journal of Cell Science, 113: 3207-3216.
73. Vanderbyl S et al (2002) Molecular Therapy, 5(5): 10.
74. Marasco W A (1997) Gene Therapy, 4(1): 11.
75. Li et al (2003). Abstract for poster [605] submitted at The American Thoracis Society Annual Meeting, 2003, Seattle.
76. Koide et al (1998). Journal of Molecular Biology, Vol 284:1141-1151.
77. Nygren et al (1997). Current Opinion in Structural Biology, Vol 7:463-469.
78. Heller, F., et al. (2002) Immunity, 17(5):629-38.
79. Darling R J, Brault P A. Kinetic Exclusion Assay Technology: Characterization of Molecular Interactions. Assay and Drug Development Technologies. 2004; Volume 2, no. 6, 647-657.
80. Rathanaswami P, Roalstad S, Roskos L, Qiaojuan J S, Lackie S, Babcook J. Demonstration of an in vivo generated sub-picomolar affinity fully human monoclonal antibody to interleukin-8. Biochemical and Biophysical Research Communications. 2005; 334:1004-1013.
81. Dall'Acqua W F et al. J Immunol. 169:171-180 (2002). Increasing the affinity of a human IgG1 for the neonatal Fc receptor: biological consequences
82. Dall'Acqua W F et al. J Biol Chem. 281:23514-24 (2006). Properties of human IgG1s engineered for enhanced binding to the neonatal Fc receptor (FcRn).

What is claimed is:

1. An isolated antigen binding protein or a fragment thereof that binds human IL-13, comprising a variable heavy (VH) domain and a variable light (VL) domain, wherein the VH domain comprises HCDR1, HCDR2 and HCDR3 and the VL domain comprises LCDR1, LCDR2 and LCDR3, and wherein:
HCDR1 comprises the amino acid sequence of SEQ ID NO: 13;
HCDR2 comprises the amino acid sequence of SEQ ID NO: 14;
HCDR3 comprises the amino acid sequence of SEQ ID NO: 15;
LCDR1 comprises the amino acid sequence having the formula:
GGNLX1LX2LX3LX4LX5LVH
wherein LX1 is selected from the group consisting of L and M,
LX2 is selected from the group consisting of L, I and V,
LX3 is selected from the group consisting of G and A,
LX4 is selected from the group consisting of S and A, and
LX5 is selected from the group consisting of R and Y (SEQ ID NO:251);
LCDR2 comprises the amino acid sequence having the formula:
DDLX6DRPS
wherein LX6 is selected from the group consisting of G, I, E, M and Q (SEQ ID NO:252); and
LCDR3 comprises the amino acid sequence having the formula:
QVWDTGSLX7PVV
wherein LX7 is selected from the group consisting of D, R, L and S (SEQ ID NO:253).

2. The antigen binding protein or fragment thereof according to claim 1, comprising a set of CDRs, HCDR1, HCDR2, HCDR3, LCDR1, LCDR2 and LCDR3, as shown in Table 3.

3. The antigen binding protein or fragment thereof according to claim 1 or 2, wherein:
LX1 is selected from the group consisting of L and M,
LX2 is selected from the group consisting of L, I and V,
LX3 is G,
LX4 is A,
LX5 is selected from the group consisting of R and Y,
LX6 is selected from the group consisting of G, I, E, M and Q, and
LX7 is selected from the group consisting of D, R, L and S.

4. The antigen binding protein or fragment thereof according to claim 3, comprising a set of CDRs, HCDR1, HCDR2, HCDR3, LCDR1, LCDR2 and LCDR3, as shown in Table 4.

5. The antigen binding protein or fragment thereof according to any one of the preceding claims, wherein:
LX1 is selected from the group consisting of L and M,
LX2 is selected from the group consisting of L, I and V,
LX3 is G,
LX4 is A,
LX5 is R,
LX6 is selected from the group consisting of G, I, E and Q, and
LX7 is selected from the group consisting of D, R, L and S.

6. The antigen binding protein or fragment thereof according to claim 5, comprising a set of CDRs, HCDR1, HCDR2, HCDR3, LCDR1, LCDR2 and LCDR3 as shown in Table 5.

7. The antigen binding protein or fragment thereof according to claim 5 or 6, wherein:
LX1 is selected from the group consisting of L and M,
LX2 is selected from the group consisting of I and V,
LX3 is G,
LX4 is A,
LX5 is R,
LX6 is selected from the group consisting of I, Q and E, and
LX7 is selected from the group consisting of R, L and S.

8. The antigen binding protein or fragment thereof according to claim 7, comprising a set of CDRs, HCDR1, HCDR2, HCDR3, LCDR1, LCDR2 and LCDR3 as shown in Table 6.

9. The antigen binding protein or fragment thereof according to claim 7 or 8, wherein:
LX1 is M,
LX2 is V,
LX3 is G,
LX4 is A,
LX5 is R,
LX6 is E, and
LX7 is S.

10. An antigen binding protein or fragment thereof according to claim 7 or 8, wherein:
LX1 is L,
LX2 is I,
LX3 is G,
LX4 is A,
LX5 is R,
LX6 is I, and
LX7 is R.

11. An antigen binding protein or fragment thereof according to claim 7 or 8, wherein:
LX1 is L,
LX2 is I,
LX3 is G,
LX4 is A,
LX5 is R,
LX6 is Q, and
LX7 is L.

12. An isolated antigen binding protein or fragment thereof that binds human IL-13 comprising a variable heavy (VH) domain and a variable light (VL) domain comprising a set of CDRs, HCDR1, HCDR2, HCDR3, LCDR1, LCDR2 and LCDR3, wherein the set of CDRs is selected from the group consisting of:
(a) HCDR1 comprising the amino acid sequence shown as SEQ ID NO: 13, HCDR2 comprising the amino acid sequence as SEQ ID NO: 14, HCDR3 comprising the amino acid sequence as SEQ ID NO: 15, LCDR1 comprising the amino acid sequence shown as SEQ ID NO: 18, LCDR2 comprising the amino acid sequence shown as SEQ ID NO: 19, and LCDR3 comprising the amino acid sequence shown as SEQ ID NO: 20;
(b) HCDR1 comprising the amino acid sequence shown as SEQ ID NO: 23, HCDR2 comprising the amino acid sequence as SEQ ID NO: 24, HCDR3 comprising the amino acid sequence as SEQ ID NO: 25, LCDR1 comprising the amino acid sequence shown as SEQ ID NO: 28, LCDR2 comprising the amino acid sequence shown as SEQ ID NO: 29, and LCDR3 comprising the amino acid sequence shown as SEQ ID NO: 30; and
(c) HCDR1 comprising the amino acid sequence shown as SEQ ID NO: 33, HCDR2 comprising the amino acid sequence shown as SEQ ID NO: 34, HCDR3 comprising the amino acid sequence shown as SEQ ID NO: 35, LCDR1 comprising the amino acid sequence shown as SEQ ID NO: 38, LCDR2 comprising the amino acid sequence shown as SEQ ID NO: 39, and LCDR3 comprising the amino acid sequence shown as SEQ ID NO: 40.

13. An isolated antigen binding protein or fragment thereof that binds IL-13, comprising a heavy chain variable region (VH) having at least 90, 95, 97, 98 or 99% sequence identity to SEQ ID NO: 12 and a light chain variable region (VL) having at least 90, 95, 97, 98 or 99% sequence identity to SEQ ID NO: 17, 27, or 37.

14. An isolated antigen binding protein or fragment thereof that binds human IL-13, comprising a VH domain and a VL domain selected from the group consisting of:
a) a VH domain comprising SEQ ID NO: 12 and a VL domain comprising SEQ ID NO: 17 (13NG0083);
(b) a VH domain comprising SEQ ID NO: 22 and a VL domain comprising SEQ ID NO: 27 (13NG0073);
(c) a VH domain comprising SEQ ID NO: 32 and a VL domain comprising SEQ ID NO: 37 (13NG0074);
(d) a VH domain comprising SEQ ID NO: 112 and a VL domain comprising SEQ ID NO: 117 (13NG0071);
(e) a VH domain comprising SEQ ID NO: 42 and a VL domain comprising SEQ ID NO: 47 (13NG0068);
(f) a VH domain comprising SEQ ID NO: 52 and a VL domain comprising SEQ ID NO: 57 (13NG0067);
(g) a VH domain comprising SEQ ID NO: 62 and a VL domain comprising SEQ ID NO: 67 (13NG0069);
(h) a VH domain comprising SEQ ID NO: 72 and a VL domain comprising SEQ ID NO: 77 (13NG0076);
(i) a VH domain comprising SEQ ID NO: 82 and a VL domain comprising SEQ ID NO: 87 (13NG0070);
(j) a VH domain comprising SEQ ID NO: 92 and a VL domain comprising SEQ ID NO: 97 (13NG0075);
(k) a VH domain comprising SEQ ID NO: 102 and a VL domain comprising SEQ ID NO: 107 (13NG0077); and
(l) a VH domain comprising SEQ ID NO: 122 and a VL domain comprising SEQ ID NO: 127 (13NG0072);
(m) a VH domain comprising SEQ ID NO: 242 and a VL domain comprising SEQ ID NO: 247 (13NG0025);
(n) a VH domain comprising SEQ ID NO: 222 and a VL domain comprising SEQ ID NO: 227 (13NG0078);
(o) a VH domain comprising SEQ ID NO: 142 and a VL domain comprising SEQ ID NO: 147 (13NG0079);
(p) a VH domain comprising SEQ ID NO: 152 and a VL domain comprising SEQ ID NO: 157 (13NG0080);
(q) a VH domain comprising SEQ ID NO: 131 and a VL domain comprising SEQ ID NO: 137 (13NG0081);
(r) a VH domain comprising SEQ ID NO: 192 and a VL domain comprising SEQ ID NO: 197 (13NG0082);
(s) a VH domain comprising SEQ ID NO: 182 and a VL domain comprising SEQ ID NO: 187 (13NG0084);
(t) a VH domain comprising SEQ ID NO: 212 and a VL domain comprising SEQ ID NO: 217 (13NG0085);
(u) a VH domain comprising SEQ ID NO: 162 and a VL domain comprising SEQ ID NO: 167 (13NG0086);
(v) a VH domain comprising SEQ ID NO: 202 and a VL domain comprising SEQ ID NO: 207 (13NG0087); and
(w) a VH domain comprising SEQ ID NO: 172 and a VL domain comprising SEQ ID NO: 177 (13NG0088).

15. The antigen binding protein or fragment thereof of claim 14, comprising a VH domain and a VL domain selected from the group consisting of:
(a) a VH domain comprising SEQ ID NO: 12 and a VL domain comprising SEQ ID NO: 17 (13NG0083);
(b) a VH domain comprising SEQ ID NO: 22 and a VL domain comprising SEQ ID NO: 27 (13NG0073); and
(c) a VH domain comprising SEQ ID NO: 32 and a VL domain comprising SEQ ID NO: 37 (13NG0074).

16. The antigen binding protein or fragment thereof according to any one of the preceding claims, wherein the HCDR1, HCDR2 and HCDR3 are within a germ-line framework and/or LCDR1, LCDR2 and LCDR3 are within a germ-line framework.

17. The antigen binding protein or fragment thereof of claim 16, wherein the HCDR1, HCDR2 and HCDR3 are within a germ-line framework comprising a set of framework regions HFW1, HFW2, HFW3 and HFW4, wherein:
HFW1 comprises an amino acid sequence having the formula:
QFX1QLVQSGAEVKKPGASVKVSCKASGYTFT,
wherein FX1 is selected from V or A (SEQ ID NO:254);
HFW2 comprises an amino acid sequence having the formula:
WVRQAPGQGLEWFX2G,
wherein FX2 is selected from M and V (SEQ ID NO:255);
HFW3 comprises an amino acid sequence having the formula:
RVTMTTDTSTFX3TAYMELRFX4LRSDDTAVYYCAR,
wherein FX3 is selected from S and G and FX4 is selected from S and G (SEQ ID NO:256); and
HFW4 comprises an amino acid sequence having the formula:

| | |
|---|---|
| W G R G T L V T V S S. | (SEQ ID NO: 257) |

18. The antigen binding protein or fragment thereof of claim 16 or 17, wherein the LCDR1, LCDR2 and LCDR3 are within a germ-line framework comprising a set of framework regions LFW1, LFW2, LFW3 and LFW4, wherein:
LFW1 comprises an amino acid sequence having the formula:
SYVLTQPPFX5VSVAPGKTARIPC,
wherein FX5 is selected from S and L (SEQ ID NO:258);
LFW2 comprises an amino acid sequence having the formula:
WYQQKPGQAPVLFX6FX7FX8,
wherein FX6 is selected from I and V,
FX7 is selected from I, M and V, and
FX8 is selected from F, Y and M (SEQ ID NO:259);
LFW3 comprises an amino acid sequence having the formula:
GIPERFSGSNSGNTATLTISRVEFX9GDEADYYC,
wherein FX9 is selected from A or T (SEQ ID NO:260); and
LFW4 comprises an amino acid sequence having the formula:

| | |
|---|---|
| F G G G T K L T V L. | (SEQ ID NO: 261) |

19. The antigen binding protein or fragment thereof of claim 18, wherein:
HFW1 comprises an amino acid sequence having the formula:
(SEQ ID NO: 262)
Q V Q L V Q S G A E V K K P G A S V K V S C K
A S G Y T F T;
HFW2 comprises an amino acid sequence having the formula:

| | |
|---|---|
| W V R Q A P G Q G L E W M G; | (SEQ ID NO: 263) |

HFW3 comprises an amino acid sequence having the formula:
(SEQ ID NO: 264)
R V T M T T D T S T S T A Y M E L R S L R S D
D T A V Y Y C A R;
HFW4 comprises an amino acid sequence having the formula:

| | |
|---|---|
| W G R G T L V T V S S; | (SEQ ID NO: 257) |

LFW1 comprises an amino acid sequence having the formula:
(SEQ ID NO: 265)
S Y V L T Q P P S V S V A P G K T A R I P C;
LFW2 comprises an amino acid sequence having the formula:

| | |
|---|---|
| W Y Q Q K P G Q A P V L I V F, | (SEQ ID NO: 266) |
| W Y Q Q K P G Q A P V L I I M, | (SEQ ID NO: 267) |
| W Y Q Q K P G Q A P V L I M F, | (SEQ ID NO: 268) |
| W Y Q Q K P G Q A P V L V I M, | (SEQ ID NO: 269) |
| W Y Q Q K P G Q A P V L I V Y, | (SEQ ID NO: 270) |
| or | |
| W Y Q Q K P G Q A P V L V I Y, | (SEQ ID NO: 271) |

LFW3 comprises an amino acid sequence having the formula:
(SEQ ID NO: 272)
G I P E R F S G S N S G N T A T L T I S R V E
A G D E A D Y Y C;
and
LFW4 comprises an amino acid sequence having the formula:

| | |
|---|---|
| F G G G T K L T V L. | (SEQ ID NO: 261) |

20. The antigen binding protein or fragment thereof of claim 19, wherein:
LFW2 comprises an amino acid sequence having the formula:
(SEQ ID NO: 266; clone 13NG0083)
W Y Q Q K P G Q A P V L I V F,
(SEQ ID NO: 267; clone 13NG0073)
W Y Q Q K P G Q A P V L I I M,
or
(SEQ ID NO: 268; clone 13NG0074)
W Y Q Q K P G Q A P V L I M F.

21. The antigen binding protein or fragment thereof according to any one of claims 16-20, wherein the HCDR1, HCDR2 and HCDR3 are within germ-line framework VH1 DP14.

22. The antigen binding protein or fragment thereof according to any one of claims 16-21, wherein the LCDR1, LCDR2 and LCDR3 are within germ-line framework VL γ3 3H.

23. An antigen binding protein, antibody, or antigen-binding fragment thereof according to anyone of the preceding claims comprising:
(1) a VL domain comprising SEQ ID NO:17 containing one or more of the substitutions selected from the group consisting of:
(a) M27I, (b) V281, (c) A30S, (d) R31K, (e) I47V, (f) V48I, (g) F49Y, (h) E52G, (i), S95A, (j) D51N, (k) E52N, (1) D53N, (m) M27I and E52N, and (n) M27I and E52G; and
(2) a VH domain comprising SEQ ID NO:12; or
a VH domain comprising a set of HCDRs HCDR1, HCDR2, and HCDR3, wherein:
HCDR1 comprises the amino acid sequence of SEQ ID NO: 13;
HCDR2 comprises the amino acid sequence of SEQ ID NO: 14; and
HCDR3 comprises the amino acid sequence of SEQ ID NO: 15.

24. The isolated antigen binding protein or fragment thereof according to any one of the preceding claims, wherein the antigen binding protein or fragment thereof has one or more properties selected from the group consisting of:
(a) Competes with a BAK1183H4 antibody for binding to IL-13, wherein the BAK1183H4 antibody comprises a VH domain comprising the amino acid sequence of SEQ ID NO: 2 and a VL domain comprising the amino acid sequence of SEQ ID NO: 7;
(b) Binds human IL-13 with an affinity better than that of the BAK1183H4 antibody, wherein the BAK1183H4 antibody comprises a VH domain comprising the amino acid sequence of SEQ ID NO: 2 and a VL domain comprising the amino acid sequence of SEQ ID NO: 7; and
(c) Binds human IL-13 with a KD value of less than about 80 pM, less than about 50 pM, less than about 20 pM, or less than about 10 pM.

25. The isolated antigen binding protein or fragment thereof of any one of the preceding claims, wherein the antigen binding protein is an antibody.

26. The isolated antigen binding protein or fragment thereof of claim 25, wherein the antibody is a monoclonal antibody, a recombinant antibody, a human antibody, a humanized antibody, a chimeric antibody, a bi-specific antibody, a multi-specific antibody, or an antibody fragment thereof.

27. The isolated antigen binding protein or fragment thereof of claim 26, wherein the antibody fragment is a Fab fragment, a Fab′ fragment, a F(ab′)₂ fragment, a Fv fragment, a diabody, or a single chain antibody molecule (scFv).

28. The antigen binding protein or fragment thereof according to any one of the preceding claims, further comprising a heavy chain immunoglobulin constant domain selected from the group consisting of:
(a) an IgA constant domain
(b) an IgD constant domain;
(c) an IgE constant domain;
(d) an IgG1 constant domain;
(e) an IgG2 constant domain;
(f) an IgG3 constant domain;
(g) an IgG4 constant domain; and
(h) an IgM constant domain.

29. The antigen binding protein or fragment thereof of claim 28, further comprising a light chain immunoglobulin constant domain selected from the group consisting of:
(a) an Ig kappa constant domain; and
(b) an Ig lambda constant domain.

30. The antigen binding protein or fragment thereof of claim 29, comprising a human IgG1 constant domain and a human lambda constant domain.

31. The antigen binding protein or fragment thereof according to any one of claims 28-30, wherein the antibody comprises an IgG1 Fc domain containing a mutation at positions 252, 254 and 256, wherein the position numbering is according to the EU index as in Kabat.

32. The antigen binding protein or fragment thereof according to claim 31, wherein the IgG1 Fc domain contains a mutation of M252Y, S254T and T256E, wherein the position numbering is according to the EU index as in Kabat.

33. The antigen binding protein or fragment thereof according to any one of the preceding claims, wherein said antigen binding protein or fragment thereof binds a human IL-13 variant in which arginine at position 130 is replaced by glutamine.

34. The antigen binding protein or fragment thereof according to any one of claims 1-31, wherein said antigen binding protein or fragment thereof binds a human IL-13 variant in which arginine at position 105 is replaced by glutamine.

35. The antigen binding protein or fragment thereof according to any one of the preceding claims which binds a non-human primate IL-13.

36. The antigen binding protein or fragment thereof according to claim 35 wherein the non-human primate IL-13 is rhesus or cynomolgus.

37. The antigen binding protein or fragment thereof according to any one of the preceding claims, that binds an epitope comprising position 106 to C-terminal asparagine at position 132 (DTKIEVAQFVKDLLLHLKKLFREGRFN (SEQ ID NO: 273)) of human IL-13 protein.

38. The antigen binding protein or fragment thereof according to any one of the preceding claims, that binds an epitope comprising phenylalanine at position 99 to C-terminal asparagine at position 132 (FSSLHVRDTKIEVAQFVKDLLLHLKKLFREGRFN (SEQ ID NO: 274)) of human IL-13 protein.

39. An isolated antibody VH domain of an antigen binding protein or fragment thereof according to any one of claims 1 to 38.

40. An isolated antibody VL domain of an antigen binding protein or fragment thereof according to any one of claims 1 to 38.

41. A composition comprising an antigen binding protein or fragment thereof, antibody VH domain or antibody VL domain of any one of the preceding claims and at least one additional component.

42. A composition according to claim 41 comprising a pharmaceutically acceptable excipient, vehicle or carrier.

43. An isolated nucleic acid encoding an antigen binding protein or fragment thereof or antibody VH or VL domain according to any one of claims 1 to 40.

44. An isolated polynucleotide or cDNA molecule sufficient for use as a hybridization probe, PCR primer or sequencing primer that is a fragment of the nucleic acid molecule of claim 43 or its complement.

45. The nucleic acid molecule according to claim 43, wherein the nucleic acid molecule is operably linked to a control sequence.

46. A vector comprising the nucleic acid molecule according to claim 45.

47. A host cell in vitro transformed with the nucleic acid of claim 43 or 45, or the vector of claim 46.

48. The host cell of claim 47, wherein the host cell is a mammalian host cell.

49. The mammalian host cell of claim 48, wherein the host cell is a NS0 murine myeloma cell, a PER.C6® human cell, or a Chinese hamster ovary (CHO) cell.

50. A hybridoma producing the antigen binding protein or fragment thereof according to any one of claims 1-38.

51. A method of making the antigen binding protein or fragment thereof of any one of claims 1-38 comprising culturing a host cell according to claims 47-49 or a hybridoma according to claim 50 under suitable conditions for producing the antigen binding protein or fragment thereof.

52. The method of claim 51 further comprising isolating the antigen binding protein or fragment thereof secreted from the host cell or hybridoma.

53. An antigen binding protein or fragment thereof produced using the method of claim 52.

54. A pharmaceutical composition comprising the antigen binding protein or fragment thereof according to any one of claim 1 to 38 or 53 and a pharmaceutically acceptable excipient.

55. The pharmaceutical composition according to claim 54 for use as a medicament.

56. Use of the pharmaceutical composition of claim 55 for treating a disease or condition associated with IL-13.

57. Use according to claim 56, wherein the disease or condition is asthma, chronic obstructive pulmonary disease (COPD), idiopathic pulmonary fibrosis (IPF), atopic dermatitis, allergic rhinitis, fibrosis, scleroderma, systemic sclerosis, pulmonary fibrosis, liver fibrosis, inflammatory bowel disease, ulcerative colitis, Sjögren's Syndrome and Hodgkin's lymphoma.

58. An antigen binding protein or fragment thereof according to any one of claim 1 to 38 or 53 or the pharmaceutical composition according to claim 54 for use in a method of treatment of a disease or condition selected from the group consisting of asthma, chronic obstructive pulmonary disease (COPD), idiopathic pulmonary fibrosis (IPF), atopic dermatitis, allergic rhinitis, fibrosis, scleroderma, systemic sclerosis, pulmonary fibrosis, liver fibrosis, inflammatory bowel disease, ulcerative colitis, Sjögren's Syndrome and Hodgkin's lymphoma.

59. A pharmaceutical composition of claim 54, further comprising a labeling group or an effector group.

60. The pharmaceutical composition of claim 59, wherein the labeling group is selected from the group consisting of: an isotopic label, a magnetic label, a redox active moiety, an optical dye, a biotinylated group and a polypeptide epitope recognized by a secondary reporter, such as GFP or biotin.

61. A pharmaceutical composition of claim 59, wherein the effector group is selected from the group consisting of a radioisotope, radionuclide, a toxin, a therapeutic and a chemotherapeutic agent.

62. A method for treating, preventing and/or ameliorating a disease or condition associated with IL-13 in a patient, comprising administering to a patient in need thereof an effective amount of a pharmaceutical composition comprising an antigen binding protein or fragment thereof according to any one of claim 1-38 or 53.

63. The method of claim 62, wherein the disease or condition is selected from the group consisting of asthma, chronic obstructive pulmonary disease (COPD), idiopathic pulmonary fibrosis (IPF), atopic dermatitis, allergic rhinitis, fibrosis, scleroderma, systemic sclerosis, pulmonary fibrosis, liver fibrosis, inflammatory bowel disease, ulcerative colitis, Sjögren's Syndrome and Hodgkin's lymphoma.

64. The method of claim 63, wherein the isolated antigen-binding protein or fragment thereof is administered alone or as a combination therapy.

65. A method of reducing IL-13 activity in a subject comprising administering an effective amount of an antigen binding protein or fragment thereof according to any one of claim 1-38 or 53 or the pharmaceutical composition according to claim 54.

66. A pharmaceutical composition comprising the antigen-binding protein or fragment thereof according to any one of claim 1 to 38 or 53 and an anti-IL-5R antibody or antigen-binding fragment thereof.

67. The pharmaceutical composition according to claim 66, wherein the anti-IL-5R antibody or antigen-binding fragment thereof comprises a VH domain comprising HCDR1, HCDR2, and HCDR3 and a VL domain comprising LCDR1, LCDR2, and LCDR3, and wherein
HCDR1 comprises the amino acid sequence of SEQ ID NO: 280;
HCDR2 comprises the amino acid sequence of SEQ ID NO: 281;
HCDR3 comprises the amino acid sequence of SEQ ID NO: 282;
LCDR1 comprises the amino acid sequence of SEQ ID NO: 283;
LCDR2 comprises the amino acid sequence of SEQ ID NO: 284; and
LCDR3 comprises the amino acid sequence of SEQ ID NO: 285.

68. The pharmaceutical composition according to claim 66 or 67, wherein anti-IL-5R antibody or antigen-binding fragment thereof comprises a VH domain comprising the amino acid sequence of SEQ ID NO:278.

69. The pharmaceutical composition according to any one of claims 66-67, wherein the anti-IL-5R antibody or antigen-binding fragment thereof comprises a VL domain comprising the amino acid sequence of SEQ ID NO:276.

70. The pharmaceutical composition according to any one of claims 66-69, wherein the anti-IL-5R antibody or antigen-binding fragment thereof comprises a VH domain comprising the amino acid sequence of SEQ ID NO:278 and a VL domain comprising the amino acid sequence of SEQ ID NO:276.

71. The pharmaceutical composition according to any one of claims 66-70, wherein the anti-IL-13 antibody or antigen-binding fragment thereof comprises a VH domain comprising HCDR1, HDR2, and HCDR3 and a VL domain comprises LCDR1, LCDR2, and LCDR3, wherein
a) HCDR1, HCDR2, and HCDR3 comprise SEQ ID NOs: 13-15, respectively, and LCDR1, LCDR2, and LCDR3 comprise SEQ ID NOs: 18-20, respectively;
b) HCDR1, HCDR2, and HCDR3 comprise SEQ ID NOs: 23-25, respectively, and LCDR1, LCDR2, and LCDR3 comprise SEQ ID NOs: 28-30, respectively; or
c) HCDR1, HCDR2, and HCDR3 comprise SEQ ID NOs: 33-35, respectively, and LCDR1, LCDR2, and LCDR3 comprise SEQ ID NOs: 38-40, respectively.

72. The pharmaceutical composition according to any one of claims 66-71, wherein the anti-IL-13 antibody or antigen-binding fragment thereof comprises
a) a VH domain comprising SEQ ID NO: 12 and a VL domain comprising SEQ ID NO:17;
b) a VH domain comprising SEQ ID NO:22 and a VL domain comprising SEQ ID NO:27; or
c) a VH domain comprising SEQ ID NO:32 and a VL domain comprising SEQ ID NO:37.

73. The method according to any one of claims 62-65, further comprising administering to the patient an anti-IL-5R antibody or antigen-binding fragment thereof.

74. The method according to claim 73, wherein the anti-IL-5R antibody or antigen-binding fragment thereof comprises a VH domain comprising HCDR1, HCDR2, and HCDR3 and a VL domain comprising LCDR1, LCDR2, and LCDR3, and wherein
HCDR1 comprises the amino acid sequence of SEQ ID NO: 280;
HCDR2 comprises the amino acid sequence of SEQ ID NO: 281;
HCDR3 comprises the amino acid sequence of SEQ ID NO: 282;
LCDR1 comprises the amino acid sequence of SEQ ID NO: 283;
LCDR2 comprises the amino acid sequence of SEQ ID NO: 284; and
LCDR3 comprises the amino acid sequence of SEQ ID NO: 285.

75. The method according to claim 73 or 74, wherein anti-IL-5R antibody or antigen-binding fragment thereof comprises a VH domain comprising the amino acid sequence of SEQ ID NO:278.

76. The method according to any one of claims 73-75, wherein the anti-IL-5R antibody or antigen-binding fragment thereof comprises a VL domain comprising the amino acid sequence of SEQ ID NO:276.

77. The method according to any one of claims 73-76, wherein the anti-IL-5R antibody or antigen-binding fragment thereof comprises a VH domain comprising the amino acid sequence of SEQ ID NO:278 and a VL domain comprising the amino acid sequence of SEQ ID NO:276.

78. The method according to any one of claims 73-77, wherein the anti-IL-13 antibody or antigen-binding fragment thereof comprises a VH domain comprising HCDR1, HDR2, and HCDR3 and a VL domain comprises LCDR1, LCDR2, and LCDR3, wherein
a) HCDR1, HCDR2, and HCDR3 comprise SEQ ID NOs: 13-15, respectively, and LCDR1, LCDR2, and LCDR3 comprise SEQ ID NOs: 18-20, respectively;
b) HCDR1, HCDR2, and HCDR3 comprise SEQ ID NOs: 23-25, respectively, and LCDR1, LCDR2, and LCDR3 comprise SEQ ID NOs: 28-30, respectively; or
c) HCDR1, HCDR2, and HCDR3 comprise SEQ ID NOs: 33-35, respectively, and LCDR1, LCDR2, and LCDR3 comprise SEQ ID NOs: 38-40, respectively.

79. The method according to any one of claims 73-78, wherein the anti-IL-13 antibody or antigen-binding fragment thereof comprises
a) a VH domain comprising SEQ ID NO: 12 and a VL domain comprising SEQ ID NO:17;
b) a VH domain comprising SEQ ID NO:22 and a VL domain comprising SEQ ID NO:27; or
c) a VH domain comprising SEQ ID NO:32 and a VL domain comprising SEQ ID NO:37.

80. The method according to any one of claims 73-79, wherein the anti-IL-13 antibody or antigen-binding fragment thereof and the anti-IL-5R antibody or antigen-binding fragment thereof are administered concurrently.

81. The method according to any one of claims 73-79, wherein the anti-IL-13 antibody or antigen-binding fragment thereof and the anti-IL-5R antibody or antigen-binding fragment thereof are administered sequentially.


SEQUENCE LISTING (433 sequences)

SEQ ID NO: 1 - DNA, 366 bp, Homo sapiens
caggtgcagctggtgcagtctggggctgaggtgaagaagcctggggcctcagtgaaggtctcctgcaaggcttctggttacacctttacaaattatggtctcagctgggtgcgacaggcccctggacaagggcttgagtggatgggatggatcaactacgacggcggcaacacacagtatggacaggaattccagggcagagtcaccatgaccacagatacatccacgagcacagcctacatggagttgaggagcctgagatctgacgacacggccgtttattactgtgcgagagactccagcagcagctgggcccgctggtttttcgatctctggggccgggggacactggtcaccgtctcgagt

SEQ ID NO: 2 - protein, 122 aa, Homo sapiens
QVQLVQSGAEVKKPGASVKVSCKASGYTFTNYGLSWVRQAPGQGLEWMGWINYDGGNTQYGQEFQGRVTMTTDTSTSTAYMELRSLRSDDTAVYYCARDSSSSWARWFFDLWGRGTLVTVSS

SEQ ID NO: 3 - protein, 5 aa, Homo sapiens
NYGLS

SEQ ID NO: 4 - protein, 17 aa, Homo sapiens
WINYDGGNTQYGQEFQG

SEQ ID NO: 5 - protein, 13 aa, Homo sapiens
DSSSSWARWFFDL

SEQ ID NO: 6 - DNA, 324 bp, Homo sapiens
tcctatgtgctgactcagccaccctcggtgtcagtggccccaggaaagacggccaggattccctgtgggggaaacatcattggaagtaaactcgtacactggtaccagcagaagccaggccaggcccctgtgctggtcatctatgatgatggcgaccggccctcagggatccctgagcgattctctggctccaactctgggaacacggccaccctgaccatcagcagggtcgaggccggggatgaggccgactattattgtcaggtgtgggatactggtagtgatcccgtggtattcggcggagggaccaagctgaccgtccta

SEQ ID NO: 7 - protein, 108 aa, Homo sapiens
SYVLTQPPSVSVAPGKTARIPCGGNIIGSKLVHWYQQKPGQAPVLVIYDDGDRPSGIPERFSGSNSGNTATLTISRVEAGDEADYYCQVWDTGSDPVVFGGGTKLTVL

SEQ ID NO: 8 - protein, 11 aa, Homo sapiens
GGNIIGSKLVH

SEQ ID NO: 9 - protein, 7 aa, Homo sapiens
DDGDRPS

SEQ ID NO: 10 - protein, 11 aa, Homo sapiens
QVWDTGSDPVV

SEQ ID NO: 11 - DNA, 366 bp, Homo sapiens; identical to SEQ ID NO: 1

SEQ ID NO: 12 - protein, 122 aa, Homo sapiens; identical to SEQ ID NO: 2

SEQ ID NO: 13 - protein, 5 aa, Homo sapiens
NYGLS

SEQ ID NO: 14 - protein, 17 aa, Homo sapiens
WINYDGGNTQYGQEFQG

SEQ ID NO: 15 - protein, 13 aa, Homo sapiens
DSSSSWARWFFDL

SEQ ID NO: 16 - DNA, 324 bp, Homo sapiens
tcctatgtgctgactcagccaccctcggtgtcagtggccccaggtaagacggccaggattccctgtgggggaaacatggtcggggcccgcctcgtccactggtaccagcagaagccaggccaggcccctgtgttgatcgtgttcgatgacgaggaccggccctcagggatccctgagcgattctctggctccaactctgggaacacggccactctgaccatcagcagggtcgaggccggggatgaggccgactattattgtcaggtgtgggatactgggagtagcccagtggtattcggcggagggaccaagctgaccgtccta

SEQ ID NO: 17 - protein, 108 aa, Homo sapiens
SYVLTQPPSVSVAPGKTARIPCGGNMVGARLVHWYQQKPGQAPVLIVFDDEDRPSGIPERFSGSNSGNTATLTISRVEAGDEADYYCQVWDTGSSPVVFGGGTKLTVL

SEQ ID NO: 18 - protein, 11 aa, Homo sapiens
GGNMVGARLVH

SEQ ID NO: 19 - protein, 7 aa, Homo sapiens
DDEDRPS

SEQ ID NO: 20 - protein, 11 aa, Homo sapiens
QVWDTGSSPVV

SEQ ID NO: 21 - DNA, 366 bp, Homo sapiens; identical to SEQ ID NO: 1

SEQ ID NO: 22 - protein, 122 aa, Homo sapiens; identical to SEQ ID NO: 2

SEQ ID NO: 23 - protein, 5 aa, Homo sapiens
NYGLS

SEQ ID NO: 24 - protein, 17 aa, Homo sapiens
WINYDGGNTQYGQEFQG

SEQ ID NO: 25 - protein, 13 aa, Homo sapiens
DSSSSWARWFFDL

SEQ ID NO: 26 - DNA, 324 bp, Homo sapiens
tcctatgtgctgactcagccaccctcggtgtcagtggccccaggtaagacggccaggattccctgtgggggaaacctgatcggggccaggctcgtccactggtatcagcagaagccaggccaggcccctgtgttgatcatcatggatgacatcgaccggccctcagggatccctgagcgattctctggctccaactctgggaacacggccaccctgaccatcagcagggtcgaggccggggatgaggccgactattattgtcaggtgtgggatactgggagtagaccagtggtattcggcggagggaccaagctgaccgtccta

SEQ ID NO: 27 - protein, 108 aa, Homo sapiens
SYVLTQPPSVSVAPGKTARIPCGGNLIGARLVHWYQQKPGQAPVLIIMDDIDRPSGIPERFSGSNSGNTATLTISRVEAGDEADYYCQVWDTGSRPVVFGGGTKLTVL

SEQ ID NO: 28 - protein, 11 aa, Homo sapiens
GGNLIGARLVH

SEQ ID NO: 29 - protein, 7 aa, Homo sapiens
DDIDRPS

SEQ ID NO: 30 - protein, 11 aa, Homo sapiens
QVWDTGSRPVV

SEQ ID NO: 31 - DNA, 366 bp, Homo sapiens; identical to SEQ ID NO: 1

SEQ ID NO: 32 - protein, 122 aa, Homo sapiens; identical to SEQ ID NO: 2

SEQ ID NO: 33 - protein, 5 aa, Homo sapiens
NYGLS

SEQ ID NO: 34 - protein, 17 aa, Homo sapiens
WINYDGGNTQYGQEFQG

SEQ ID NO: 35 - protein, 13 aa, Homo sapiens
DSSSSWARWFFDL

SEQ ID NO: 36 - DNA, 324 bp, Homo sapiens
tcctatgtgctgactcagccaccctcggtgtcagtggccccaggtaagacggccaggattccctgtgggggaaacctgatcggggccaggctcgtccactggtatcagcagaagccaggccaggcccctgtgctcatcatgttcgatgaccaggaccggccctcagggatccctgagcgattctctggctccaactctgggaacacggccaccctgaccatcagcagggtcgaggccggggatgaggccgactattattgtcaggtgtgggatactgggagtttgccggtggtattcggcggagggaccaagctgaccgtccta

SEQ ID NO: 37 - protein, 108 aa, Homo sapiens
SYVLTQPPSVSVAPGKTARIPCGGNLIGARLVHWYQQKPGQAPVLIMFDDQDRPSGIPERFSGSNSGNTATLTISRVEAGDEADYYCQVWDTGSLPVVFGGGTKLTVL

SEQ ID NO: 38 - protein, 11 aa, Homo sapiens
GGNLIGARLVH

SEQ ID NO: 39 - protein, 7 aa, Homo sapiens
DDQDRPS

SEQ ID NO: 40 - protein, 11 aa, Homo sapiens
QVWDTGSLPVV

SEQ ID NO: 41 - DNA, 366 bp, Homo sapiens
caggtgcagctggtgcagtctggggctgaggtgaagaagcctggggcctcagtgaaggtctcctgcaaggcttctggttacacctttacaaattatggtctcagctgggtgcgacaggcccctggacaagggcttgagtgggtgggatggatcaactacgacggcggcaacacacagtatggacaggaattccagggcagagtcaccatgaccacagatacatccacgggcacagcctacatggagttgaggagcctgagatctgacgacacggccgtttattactgtgcgagagactccagcagcagctgggcccgctggtttttcgatctctggggccgggggacactggtcaccgtctcgagt

SEQ ID NO: 42 - protein, 122 aa, Homo sapiens
QVQLVQSGAEVKKPGASVKVSCKASGYTFTNYGLSWVRQAPGQGLEWVGWINYDGGNTQYGQEFQGRVTMTTDTSTGTAYMELRSLRSDDTAVYYCARDSSSSWARWFFDLWGRGTLVTVSS

SEQ ID NO: 43 - protein, 5 aa, Homo sapiens
NYGLS

SEQ ID NO: 44 - protein, 17 aa, Homo sapiens
WINYDGGNTQYGQEFQG

SEQ ID NO: 45 - protein, 13 aa, Homo sapiens
DSSSSWARWFFDL

SEQ ID NO: 46 - DNA, 324 bp, Homo sapiens
tcctatgtgctgactcagccacccttggtgtcagtggccccaggtaagacggccaggattccctgtgggggaaacctgatcggggccaggctcgtccactggtaccagcagaagccaggccaggcccctgtgttgatcatcatggatgacatcgaccggccctcagggatccctgagcgattctctggctccaactctgggaacacggccaccctgaccatcagcagggtcgaggccggggatgaggccgactattattgtcaggtgtgggatactggtagtgatcccgtggtattcggcggagggaccaagctgaccgtccta

SEQ ID NO: 47 - protein, 108 aa, Homo sapiens
SYVLTQPPLVSVAPGKTARIPCGGNLIGARLVHWYQQKPGQAPVLIIMDDIDRPSGIPERFSGSNSGNTATLTISRVEAGDEADYYCQVWDTGSDPVVFGGGTKLTVL

SEQ ID NO: 48 - protein, 11 aa, Homo sapiens
GGNLIGARLVH

SEQ ID NO: 49 - protein, 7 aa, Homo sapiens
DDIDRPS

SEQ ID NO: 50 - protein, 11 aa, Homo sapiens
QVWDTGSDPVV

SEQ ID NO: 51 - DNA, 366 bp, Homo sapiens
caggtgcagctggtgcagtctggggctgaggtgaagaagcctggggcctcagtgaaggtctcctgcaaggcttctggttacacctttacaaattatggtctcagctgggtgcgacaggcccctggacaagggcttgagtggatgggatggatcaactacgacggcggcaacacacagtatggacaggaattccagggcagagtcaccatgaccacagatacatccacgagcacagcctacatggagttgaggggcctgagatctgacgacacggccgtttattactgtgcgagagactccagcagcagctgggcccgctggtttttcgatctctggggccgggggacactggtcaccgtctcgagt

SEQ ID NO: 52 - protein, 122 aa, Homo sapiens
QVQLVQSGAEVKKPGASVKVSCKASGYTFTNYGLSWVRQAPGQGLEWMGWINYDGGNTQYGQEFQGRVTMTTDTSTSTAYMELRGLRSDDTAVYYCARDSSSSWARWFFDLWGRGTLVTVSS

SEQ ID NO: 53 - protein, 5 aa, Homo sapiens
NYGLS

SEQ ID NO: 54 - protein, 17 aa, Homo sapiens
WINYDGGNTQYGQEFQG

SEQ ID NO: 55 - protein, 13 aa, Homo sapiens
DSSSSWARWFFDL

SEQ ID NO: 56 - DNA, 324 bp, Homo sapiens
tcctatgtgctgactcagccaccctcggtgtcagtggccccaggtaagacggccaggattccctgtgggggaaacatggtcggggcccgcctcgtccactggtatcagcagaagccaggccaggcccctgtgttgatcatcatggatgacatcgaccggccctcagggatccctgagcgattctctggctccaactctgggaacacggccaccctgaccatcagcagggtcgaggccggggatgaggccgactattattgtcaggtgtgggatactgggagtagcccagtggtattcggcggagggaccaagctgaccgtccta

SEQ ID NO: 57 - protein, 108 aa, Homo sapiens
SYVLTQPPSVSVAPGKTARIPCGGNMVGARLVHWYQQKPGQAPVLIIMDDIDRPSGIPERFSGSNSGNTATLTISRVEAGDEADYYCQVWDTGSSPVVFGGGTKLTVL

SEQ ID NO: 58 - protein, 11 aa, Homo sapiens
GGNMVGARLVH

SEQ ID NO: 59 - protein, 7 aa, Homo sapiens
DDIDRPS

SEQ ID NO: 60 - protein, 11 aa, Homo sapiens
QVWDTGSSPVV

SEQ ID NO: 61 - DNA, 366 bp, Homo sapiens; identical to SEQ ID NO: 1

SEQ ID NO: 62 - protein, 122 aa, Homo sapiens; identical to SEQ ID NO: 2

SEQ ID NO: 63 - protein, 5 aa, Homo sapiens
NYGLS

SEQ ID NO: 64 - protein, 17 aa, Homo sapiens
WINYDGGNTQYGQEFQG

SEQ ID NO: 65 - protein, 13 aa, Homo sapiens
DSSSSWARWFFDL

SEQ ID NO: 66 - DNA, 324 bp, Homo sapiens
tcctatgtgctgactcagccaccctcggtgtcagtggccccaggtaagacggccaggattccctgtgggggaaacatggtcggggcccgcctcgtccactggtaccagcagaagccaggccaggcccctgtgttgatcatcatggatgacatcgaccggccctcagggatccctgagcgattctctggctccaactctgggaacacggccaccctgaccatcagcagggtcgaggccggggatgaggccgactattattgtcaggtgtgggatactgggagtagaccagtggtattcggcggagggaccaagctgaccgtccta

SEQ ID NO: 67 - protein, 108 aa, Homo sapiens
SYVLTQPPSVSVAPGKTARIPCGGNMVGARLVHWYQQKPGQAPVLIIMDDIDRPSGIPERFSGSNSGNTATLTISRVEAGDEADYYCQVWDTGSRPVVFGGGTKLTVL

SEQ ID NO: 68 - protein, 11 aa, Homo sapiens
GGNMVGARLVH

SEQ ID NO: 69 - protein, 7 aa, Homo sapiens
DDIDRPS

SEQ ID NO: 70 - protein, 11 aa, Homo sapiens
QVWDTGSRPVV

SEQ ID NO: 71 - DNA, 366 bp, Homo sapiens; identical to SEQ ID NO: 1

SEQ ID NO: 72 - protein, 122 aa, Homo sapiens; identical to SEQ ID NO: 2

SEQ ID NO: 73 - protein, 5 aa, Homo sapiens
NYGLS

SEQ ID NO: 74 - protein, 17 aa, Homo sapiens
WINYDGGNTQYGQEFQG

SEQ ID NO: 75 - protein, 13 aa, Homo sapiens
DSSSSWARWFFDL

SEQ ID NO: 76 - DNA, 324 bp, Homo sapiens
tcctatgtgctgactcagccaccctcggtgtcagtggccccaggtaagacggccaggattccctgtgggggaaacatggtcggggcccgcctcgtccactggtaccagcagaagccaggccaggcccctgtgctggtcatcatggatgacatcgaccggccctcagggatccctgagcgattctctggctccaactctgggaacacggccaccctgaccatcagcagggtcgaggccggggatgaggccgactattattgtcaggtgtgggatactgggagtagaccagtggtattcggcggagggaccaagctgaccgtccta

SEQ ID NO: 77 - protein, 108 aa, Homo sapiens
SYVLTQPPSVSVAPGKTARIPCGGNMVGARLVHWYQQKPGQAPVLVIMDDIDRPSGIPERFSGSNSGNTATLTISRVEAGDEADYYCQVWDTGSRPVVFGGGTKLTVL

SEQ ID NO: 78 - protein, 11 aa, Homo sapiens
GGNMVGARLVH

SEQ ID NO: 79 - protein, 7 aa, Homo sapiens
DDIDRPS

SEQ ID NO: 80 - protein, 11 aa, Homo sapiens
QVWDTGSRPVV

SEQ ID NO: 81 - DNA, 366 bp, Homo sapiens; identical to SEQ ID NO: 1

SEQ ID NO: 82 - protein, 122 aa, Homo sapiens; identical to SEQ ID NO: 2

SEQ ID NO: 83 - protein, 5 aa, Homo sapiens
NYGLS

SEQ ID NO: 84 - protein, 17 aa, Homo sapiens
WINYDGGNTQYGQEFQG

SEQ ID NO: 85 - protein, 13 aa, Homo sapiens
DSSSSWARWFFDL

SEQ ID NO: 86 - DNA, 324 bp, Homo sapiens
tcctatgtgctgactcagccaccctcggtgtcagtggccccaggtaagacggccaggattccctgtgggggaaacatggtcggggcccgcctcgtccactggtatcagcagaagccaggccaggcccctgtgttgatcgtgttcgatgacgaggaccggccctcagggatccctgagcgattctctggctccaactctgggaacacggccactctgaccatcagcagggtcgaggccggggatgaggccgactattattgtcaggtgtgggatactgggagtagaccagtggtattcggcggagggaccaagctgaccgtccta

SEQ ID NO: 87 - protein, 108 aa, Homo sapiens
SYVLTQPPSVSVAPGKTARIPCGGNMVGARLVHWYQQKPGQAPVLIVFDDEDRPSGIPERFSGSNSGNTATLTISRVEAGDEADYYCQVWDTGSRPVVFGGGTKLTVL

SEQ ID NO: 88 - protein, 11 aa, Homo sapiens
GGNMVGARLVH

SEQ ID NO: 89 - protein, 7 aa, Homo sapiens
DDEDRPS

SEQ ID NO: 90 - protein, 11 aa, Homo sapiens
QVWDTGSRPVV

SEQ ID NO: 91 - DNA, 366 bp, Homo sapiens; identical to SEQ ID NO: 1

SEQ ID NO: 92 - protein, 122 aa, Homo sapiens; identical to SEQ ID NO: 2

SEQ ID NO: 93 - protein, 5 aa, Homo sapiens
NYGLS

SEQ ID NO: 94 - protein, 17 aa, Homo sapiens
WINYDGGNTQYGQEFQG

SEQ ID NO: 95 - protein, 13 aa, Homo sapiens
DSSSSWARWFFDL

SEQ ID NO: 96 - DNA, 324 bp, Homo sapiens
tcctatgtgctgactcagccaccctcggtgtcagtggccccaggtaagacggccaggattccctgtgggggaaacatggtcggggcccgcctcgtccactggtatcagcagaagccaggccaggcccctgtgctggtcatcatggatgacatcgaccggccctcagggatccctgagcgattctctggctccaactctgggaacacggccactctgaccatcagcagggtcgaggccggggatgaggccgactattattgtcaggtgtgggatactgggagtagaccagtggtattcggcggagggaccaagctgaccgtccta

SEQ ID NO: 97 - protein, 108 aa, Homo sapiens; identical to SEQ ID NO: 77

SEQ ID NO: 98 - protein, 11 aa, Homo sapiens
GGNMVGARLVH

SEQ ID NO: 99 - protein, 7 aa, Homo sapiens
DDIDRPS

SEQ ID NO: 100 - protein, 11 aa, Homo sapiens
QVWDTGSRPVV

SEQ ID NO: 101 - DNA, 366 bp, Homo sapiens
caggtgcagctggtgcagtctggggctgaggtgaagaagcctggggcctcagtgaaggtctcctgcaaggcttctggttacacctttacaaattatggtctcagctgggtgcgacaggcccctggacaagggcttgagtggatgggatggatcaactacgacggcggcaacacacagtatggacaggaattccagggcagagtcaccatgaccacagatacatccacaagcacagcctacatggagttgaggagcctgagatctgacgacacggccgtttattactgtgcgagagactccagcagcagctgggcccgctggtttttcgatctctggggccgggggacactggtcaccgtctcgagt

SEQ ID NO: 102 - protein, 122 aa, Homo sapiens; identical to SEQ ID NO: 2

SEQ ID NO: 103 - protein, 5 aa, Homo sapiens
NYGLS

SEQ ID NO: 104 - protein, 17 aa, Homo sapiens
WINYDGGNTQYGQEFQG

SEQ ID NO: 105 - protein, 13 aa, Homo sapiens
DSSSSWARWFFDL

SEQ ID NO: 106 - DNA, 324 bp, Homo sapiens
tcctatgtgctgactcagccaccctcggtgtcagtggccccaggtaagacggccaggattccctgtgggggaaacatggtgggggcgtacctcgtgcactggtaccagcagaagccaggccaggcccctgtgctgatcgtgtacgatgacatggaccggccctcagggatccctgagcgattctctggctccaactctgggaacacggccactctgaccatcagcagggtcgaggccggggatgaggccgactattattgtcaggtgtgggatactgggagtagcccagtggtattcggcggagggaccaagctgaccgtccta

SEQ ID NO: 107 - protein, 108 aa, Homo sapiens
SYVLTQPPSVSVAPGKTARIPCGGNMVGAYLVHWYQQKPGQAPVLIVYDDMDRPSGIPERFSGSNSGNTATLTISRVEAGDEADYYCQVWDTGSSPVVFGGGTKLTVL

SEQ ID NO: 108 - protein, 11 aa, Homo sapiens
GGNMVGAYLVH

SEQ ID NO: 109 - protein, 7 aa, Homo sapiens
DDMDRPS

SEQ ID NO: 110 - protein, 11 aa, Homo sapiens
QVWDTGSSPVV

SEQ ID NO: 111 - DNA, 366 bp, Homo sapiens; identical to SEQ ID NO: 1

SEQ ID NO: 112 - protein, 122 aa, Homo sapiens; identical to SEQ ID NO: 2

SEQ ID NO: 113 - protein, 5 aa, Homo sapiens
NYGLS

SEQ ID NO: 114 - protein, 17 aa, Homo sapiens
WINYDGGNTQYGQEFQG

SEQ ID NO: 115 - protein, 13 aa, Homo sapiens
DSSSSWARWFFDL

SEQ ID NO: 116 - DNA, 324 bp, Homo sapiens
tcctatgtgctgactcagccaccctcggtgtcagtggccccaggtaagacggccaggattccctgtgggggaaacatggtcggggcccgcctcgtccactggtatcagcagaagccaggccaggcccctgtgttgatcgtgttcgatgacgaggaccggccctcagggatccctgagcgattctctggctccaactctgggaacacggccaccctgaccatcagcagggtcgaggccggggatgaggccgactattattgtcaggtgtgggatactgggagtttgccggtggtattcggcggagggaccaagctgaccgtccta

SEQ ID NO: 117 - protein, 108 aa, Homo sapiens
SYVLTQPPSVSVAPGKTARIPCGGNMVGARLVHWYQQKPGQAPVLIVFDDEDRPSGIPERFSGSNSGNTATLTISRVEAGDEADYYCQVWDTGSLPVVFGGGTKLTVL

SEQ ID NO: 118 - protein, 11 aa, Homo sapiens
GGNMVGARLVH

SEQ ID NO: 119 - protein, 7 aa, Homo sapiens
DDEDRPS

SEQ ID NO: 120 - protein, 11 aa, Homo sapiens
QVWDTGSLPVV

SEQ ID NO: 121 - DNA, 366 bp, Homo sapiens; identical to SEQ ID NO: 1

SEQ ID NO: 122 - protein, 122 aa, Homo sapiens; identical to SEQ ID NO: 2

SEQ ID NO: 123 - protein, 5 aa, Homo sapiens
NYGLS

SEQ ID NO: 124 - protein, 17 aa, Homo sapiens
WINYDGGNTQYGQEFQG

SEQ ID NO: 125 - protein, 13 aa, Homo sapiens
DSSSSWARWFFDL

SEQ ID NO: 126 - DNA, 324 bp, Homo sapiens
tcctatgtgctgactcagccaccctcggtgtcagtggccccaggtaagacggccaggattccctgtgggggaaacatggtcggggcccgcctcgtccactggtatcagcagaagccaggccaggcccctgtgctgatcgtgtacgatgacatggaccggccctcagggatccctgagcgattctctggctccaactctgggaacacggccaccctgaccatcagcagggtcgagaccggggatgaggccgactattattgtcaggtgtgggatactgggagtagaccagtggtattcggcggagggaccaagctgaccgtccta

SEQ ID NO: 127 - protein, 108 aa, Homo sapiens
SYVLTQPPSVSVAPGKTARIPCGGNMVGARLVHWYQQKPGQAPVLIVYDDMDRPSGIPERFSGSNSGNTATLTISRVETGDEADYYCQVWDTGSRPVVFGGGTKLTVL

SEQ ID NO: 128 - protein, 11 aa, Homo sapiens
GGNMVGARLVH

SEQ ID NO: 129 - protein, 7 aa, Homo sapiens
DDMDRPS

SEQ ID NO: 130 - protein, 11 aa, Homo sapiens
QVWDTGSRPVV

SEQ ID NO: 131 - DNA, 366 bp, Homo sapiens
caggtgcagctggtgcagtctggggctgaggtgaagaagcctggggcctcagtgaaggtctcctgcaaggcttctggttacacctttacaaattatggtctcagctgggtgcgacaggcccctggacaagggcttgagtgggtgggatggatcaactacgacggcggcaacacacagtatggacaggaattccagggcagagtcaccatgaccacagatacatccacgagcacagcctacatggagttgaggagcctgagatctgacgacacggccgtttattactgtgcgagagactccagcagcagctgggcccgctggtttttcgatctctggggccgggggacactggtcaccgtctcgagt

SEQ ID NO: 132 - protein, 122 aa, Homo sapiens
QVQLVQSGAEVKKPGASVKVSCKASGYTFTNYGLSWVRQAPGQGLEWVGWINYDGGNTQYGQEFQGRVTMTTDTSTSTAYMELRSLRSDDTAVYYCARDSSSSWARWFFDLWGRGTLVTVSS

SEQ ID NO: 133 - protein, 5 aa, Homo sapiens
NYGLS

SEQ ID NO: 134 - protein, 17 aa, Homo sapiens
WINYDGGNTQYGQEFQG

SEQ ID NO: 135 - protein, 13 aa, Homo sapiens
DSSSSWARWFFDL

SEQ ID NO: 136 - DNA, 324 bp, Homo sapiens
tcctatgtgctgactcagccaccctcggtgtcagtggccccaggtaagacggccaggattccctgtgggggaaacctgatcggggccaggctcgtccactggtaccagcagaagccaggccaggcccctgtgctgatcgtgtacgatgacatggaccggccctcagggatccctgagcgattctctggctccaactctgggaacacggccaccctgaccatcagcagggtcgaggccggggatgaggccgactattattgtcaggtgtgggatactgggagtagaccagtggtattcggcggagggaccaagctgaccgtccta

SEQ ID NO: 137 - protein, 108 aa, Homo sapiens
SYVLTQPPSVSVAPGKTARIPCGGNLIGARLVHWYQQKPGQAPVLIVYDDMDRPSGIPERFSGSNSGNTATLTISRVEAGDEADYYCQVWDTGSRPVVFGGGTKLTVL

SEQ ID NO: 138 - protein, 11 aa, Homo sapiens
GGNLIGARLVH

SEQ ID NO: 139 - protein, 7 aa, Homo sapiens
DDMDRPS

SEQ ID NO: 140 - protein, 11 aa, Homo sapiens
QVWDTGSRPVV

SEQ ID NO: 141 - DNA, 366 bp, Homo sapiens; identical to SEQ ID NO: 1

SEQ ID NO: 142 - protein, 122 aa, Homo sapiens; identical to SEQ ID NO: 2

SEQ ID NO: 143 - protein, 5 aa, Homo sapiens
NYGLS

SEQ ID NO: 144 - protein, 17 aa, Homo sapiens
WINYDGGNTQYGQEFQG

SEQ ID NO: 145 - protein, 13 aa, Homo sapiens
DSSSSWARWFFDL

SEQ ID NO: 146 - DNA, 324 bp, Homo sapiens
tcctatgtgctgactcagccaccctcggtgtcagtggccccaggtaagacggccaggattccctgtgggggaaacctgatcggggccaggctcgtccactggtaccagcagaagccaggccaggcccctgtgctgatcgtgtacgatgacatggaccggccctcagggatccctgagcgattctctggctccaactctgggaacacggccaccctgaccatcagcagggtcgaggccggggatgaggccgactattattgtcaggtgtgggatactgggagtttgccggtggtattcggcggagggaccaagctgaccgtccta

SEQ ID NO: 147 - protein, 108 aa, Homo sapiens
SYVLTQPPSVSVAPGKTARIPCGGNLIGARLVHWYQQKPGQAPVLIVYDDMDRPSGIPERFSGSNSGNTATLTISRVEAGDEADYYCQVWDTGSLPVVFGGGTKLTVL

SEQ ID NO: 148 - protein, 11 aa, Homo sapiens
GGNLIGARLVH

SEQ ID NO: 149 - protein, 7 aa, Homo sapiens
DDMDRPS

SEQ ID NO: 150 - protein, 11 aa, Homo sapiens
QVWDTGSLPVV

SEQ ID NO: 151 - DNA, 366 bp, Homo sapiens
caggcgcagctggtgcagtctggggctgaggtgaagaagcctggggcctcagtgaaggtctcctgcaaggcttctggttacacctttacaaattatggtctcagctgggtgcgacaggcccctggacaagggcttgagtggatgggatggatcaactacgacggcggcaacacacagtatggacaggaattccagggcagagtcaccatgaccacagatacatccacgagcacagcctacatggagttgaggagcctgagatctgacgacacggccgtttattactgtgcgagagactccagcagcagctgggcccgctggtttttcgatctctggggccgggggacactggtcaccgtctcgagt

SEQ ID NO: 152 - protein, 122 aa, Homo sapiens
QAQLVQSGAEVKKPGASVKVSCKASGYTFTNYGLSWVRQAPGQGLEWMGWINYDGGNTQYGQEFQGRVTMTTDTSTSTAYMELRSLRSDDTAVYYCARDSSSSWARWFFDLWGRGTLVTVSS

SEQ ID NO: 153 - protein, 5 aa, Homo sapiens
NYGLS

SEQ ID NO: 154 - protein, 17 aa, Homo sapiens
WINYDGGNTQYGQEFQG

SEQ ID NO: 155 - protein, 13 aa, Homo sapiens
DSSSSWARWFFDL

SEQ ID NO: 156 - DNA, 324 bp, Homo sapiens
tcctatgtgctgactcagccaccctcggtgtcagtggccccaggtaagacggccaggattccctgtgggggaaacttgatcgcggcccgcctcgtccattggtatcagcagaagccaggccaggcccctgtgttgatcgtgttcgatgacgaggaccggccctcagggatccctgagcgattctctggctccaactctgggaacacggccaccctgaccatcagcagggtcgagaccggggatgaggccgactattattgtcaggtgtgggatactgggagtagcccagtggtattcggcggagggaccaagctgaccgtccta

SEQ ID NO: 157 - protein, 108 aa, Homo sapiens
SYVLTQPPSVSVAPGKTARIPCGGNLIAARLVHWYQQKPGQAPVLIVFDDEDRPSGIPERFSGSNSGNTATLTISRVETGDEADYYCQVWDTGSSPVVFGGGTKLTVL

SEQ ID NO: 158 - protein, 11 aa, Homo sapiens
GGNLIAARLVH

SEQ ID NO: 159 - protein, 7 aa, Homo sapiens
DDEDRPS

SEQ ID NO: 160 - protein, 11 aa, Homo sapiens
QVWDTGSSPVV

SEQ ID NO: 161 - DNA, 366 bp, Homo sapiens; identical to SEQ ID NO: 1

SEQ ID NO: 162 - protein, 122 aa, Homo sapiens; identical to SEQ ID NO: 2

SEQ ID NO: 163 - protein, 5 aa, Homo sapiens
NYGLS

SEQ ID NO: 164 - protein, 17 aa, Homo sapiens
WINYDGGNTQYGQEFQG

SEQ ID NO: 165 - protein, 13 aa, Homo sapiens
DSSSSWARWFFDL

SEQ ID NO: 166 - DNA, 324 bp, Homo sapiens
tcctatgtgctgactcagccaccctcggtgtcagtggccccaggtaagacggccaggattccctgtgggggaaacatggtcggggcccgcctcgtccactggtaccagcagaagccaggccaggcccctgtgctgatcgtgtacgatgacatggaccggccctcagggatccctgagcgattctctggctccaactctgggaacacggccactctgaccatcagcagggtcgaggccggggatgaggccgactattattgtcaggtgtgggatactgggagtagaccagtggtattcggcggagggaccaagctgaccgtccta

SEQ ID NO: 167 - protein, 108 aa, Homo sapiens
SYVLTQPPSVSVAPGKTARIPCGGNMVGARLVHWYQQKPGQAPVLIVYDDMDRPSGIPERFSGSNSGNTATLTISRVEAGDEADYYCQVWDTGSRPVVFGGGTKLTVL

SEQ ID NO: 168 - protein, 11 aa, Homo sapiens
GGNMVGARLVH

SEQ ID NO: 169 - protein, 7 aa, Homo sapiens
DDMDRPS

SEQ ID NO: 170 - protein, 11 aa, Homo sapiens
QVWDTGSRPVV

SEQ ID NO: 171 - DNA, 366 bp, Homo sapiens
caggtgcagctggtgcagtctggggctgaggtgaagaagcctggggcctcagtgaaggtctcctgcaaggcttctggttacacctttacaaattatggtctcagctgggtgcgacaggcccctggacaagggcttgagtggatgggatggatcaactacgacggcggcaacacacagtatggacaggaattccagggcagagtcaccatgaccacagatacatccacgagcacagcctacatggagttgaggagcctgagatctgatgacacggccgtttattactgtgcgagagactccagcagcagctgggcccgctggtttttcgatctctggggccgggggacactggtcaccgtctcgagt

SEQ ID NO: 172 - protein, 122 aa, Homo sapiens; identical to SEQ ID NO: 2

SEQ ID NO: 173 - protein, 5 aa, Homo sapiens
NYGLS

SEQ ID NO: 174 - protein, 17 aa, Homo sapiens
WINYDGGNTQYGQEFQG

SEQ ID NO: 175 - protein, 13 aa, Homo sapiens
DSSSSWARWFFDL

SEQ ID NO: 176 - DNA, 324 bp, Homo sapiens
tcctatgtgctgactcagccaccctcggtgtcagtggccccaggtaagacggccaggattccctgtggaggaaacctgatcggggccaggctcgtccactggtaccagcagaagccaggccaggcccctgtgctggtcatcatggatgacatcgaccggccctcagggatccctgagcgattctctggctccaactctgggaacacggccactctgaccatcagcagggtcgaggccggggatgaggccgactattattgtcaggtgtgggatactgggagtagaccagtggtattcggcggagggaccaagctgaccgtccta

SEQ ID NO: 177 - protein, 108 aa, Homo sapiens
SYVLTQPPSVSVAPGKTARIPCGGNLIGARLVHWYQQKPGQAPVLVIMDDIDRPSGIPERFSGSNSGNTATLTISRVEAGDEADYYCQVWDTGSRPVVFGGGTKLTVL

SEQ ID NO: 178 - protein, 11 aa, Homo sapiens
GGNLIGARLVH

SEQ ID NO: 179 - protein, 7 aa, Homo sapiens
DDIDRPS

SEQ ID NO: 180 - protein, 11 aa, Homo sapiens
QVWDTGSRPVV

SEQ ID NO: 181 - DNA, 366 bp, Homo sapiens; identical to SEQ ID NO: 1

SEQ ID NO: 182 - protein, 122 aa, Homo sapiens; identical to SEQ ID NO: 2

SEQ ID NO: 183 - protein, 5 aa, Homo sapiens
NYGLS

SEQ ID NO: 184 - protein, 17 aa, Homo sapiens
WINYDGGNTQYGQEFQG

SEQ ID NO: 185 - protein, 13 aa, Homo sapiens
DSSSSWARWFFDL

SEQ ID NO: 186 - DNA, 324 bp, Homo sapiens
tcctatgtgctgactcagccaccctcggtgtcagtggccccaggtaagacggccaggattccctgtgggggaaacttgatcgcggcccgcctcgtccattggtatcagcagaagccaggccaggcccctgtgttgatcgtgttcgatgacgaggaccggccctcagggatccctgagcgattctctggctccaactctgggaacacggccaccctgaccatcagcagggtcgaggccggggatgaggccgactattattgtcaggtgtgggatactgggagtagcccagtggtattcggcggagggaccaagctgaccgtccta

SEQ ID NO: 187 - protein, 108 aa, Homo sapiens
SYVLTQPPSVSVAPGKTARIPCGGNLIAARLVHWYQQKPGQAPVLIVFDDEDRPSGIPERFSGSNSGNTATLTISRVEAGDEADYYCQVWDTGSSPVVFGGGTKLTVL

SEQ ID NO: 188 - protein, 11 aa, Homo sapiens
GGNLIAARLVH

SEQ ID NO: 189 - protein, 7 aa, Homo sapiens
DDEDRPS

SEQ ID NO: 190 - protein, 11 aa, Homo sapiens
QVWDTGSSPVV

SEQ ID NO: 191 - DNA, 366 bp, Homo sapiens; identical to SEQ ID NO: 1

SEQ ID NO: 192 - protein, 122 aa, Homo sapiens; identical to SEQ ID NO: 2

SEQ ID NO: 193 - protein, 5 aa, Homo sapiens
NYGLS

SEQ ID NO: 194 - protein, 17 aa, Homo sapiens
WINYDGGNTQYGQEFQG

SEQ ID NO: 195 - protein, 13 aa, Homo sapiens
DSSSSWARWFFDL

SEQ ID NO: 196 - DNA, 324 bp, Homo sapiens
tcctatgtgctgactcagccaccctcggtgtcagtggccccaggtaagacggccaggattccctgtgggggaaacatggtcggggcccgcctcgtccactggtaccagcagaagccaggccaggcccctgtgttgatcgtgttcgatgacgaggaccggccctcagggatccctgagcgattctctggctccaactctgggaacacggccaccctgaccatcagcagggtcgaggccggggatgaggccgactattattgtcaggtgtgggatactgggagtagaccagtggtattcggcggagggaccaagctgaccgtccta

SEQ ID NO: 197 - protein, 108 aa, Homo sapiens; identical to SEQ ID NO: 87

SEQ ID NO: 198 - protein, 11 aa, Homo sapiens
GGNMVGARLVH

SEQ ID NO: 199 - protein, 7 aa, Homo sapiens
DDEDRPS

SEQ ID NO: 200 - protein, 11 aa, Homo sapiens
QVWDTGSRPVV

SEQ ID NO: 201 - DNA, 366 bp, Homo sapiens; identical to SEQ ID NO: 1

SEQ ID NO: 202 - protein, 122 aa, Homo sapiens; identical to SEQ ID NO: 2

SEQ ID NO: 203 - protein, 5 aa, Homo sapiens
NYGLS

SEQ ID NO: 204 - protein, 17 aa, Homo sapiens
WINYDGGNTQYGQEFQG

SEQ ID NO: 205 - protein, 13 aa, Homo sapiens
DSSSSWARWFFDL

SEQ ID NO: 206 - DNA, 324 bp, Homo sapiens
tcctatgtgctgactcagccaccctcggtgtcagtggccccaggtaagacggccaggattccctgtgggggaaacatggtggcggccaggctcgtccactggtaccagcagaagccaggccaggcccctgtgctcatcatgttcgatgaccaggaccggccctcagggatccctgagcgattctctggctccaactctgggaacacggccacactgaccatcagcagggtcgaggccggggatgaggccgactattattgtcaggtgtgggatactgggagtttgccggtggtattcggcggagggaccaagctgaccgtccta

SEQ ID NO: 207 - protein, 108 aa, Homo sapiens
SYVLTQPPSVSVAPGKTARIPCGGNMVAARLVHWYQQKPGQAPVLIMFDDQDRPSGIPERFSGSNSGNTATLTISRVEAGDEADYYCQVWDTGSLPVVFGGGTKLTVL

SEQ ID NO: 208 - protein, 11 aa, Homo sapiens
GGNMVAARLVH

SEQ ID NO: 209 - protein, 7 aa, Homo sapiens
DDQDRPS

SEQ ID NO: 210 - protein, 11 aa, Homo sapiens
QVWDTGSLPVV

SEQ ID NO: 211 - DNA, 366 bp, Homo sapiens; identical to SEQ ID NO: 1

SEQ ID NO: 212 - protein, 122 aa, Homo sapiens; identical to SEQ ID NO: 2

SEQ ID NO: 213 - protein, 5 aa, Homo sapiens
NYGLS

SEQ ID NO: 214 - protein, 17 aa, Homo sapiens
WINYDGGNTQYGQEFQG

SEQ ID NO: 215 - protein, 13 aa, Homo sapiens
DSSSSWARWFFDL

SEQ ID NO: 216 - DNA, 324 bp, Homo sapiens; identical to SEQ ID NO: 66

SEQ ID NO: 217 - protein, 108 aa, Homo sapiens; identical to SEQ ID NO: 67

SEQ ID NO: 218 - protein, 11 aa, Homo sapiens
GGNMVGARLVH

SEQ ID NO: 219 - protein, 7 aa, Homo sapiens
DDIDRPS

SEQ ID NO: 220 - protein, 11 aa, Homo sapiens
QVWDTGSRPVV

SEQ ID NO: 221 - DNA, 366 bp, Homo sapiens; identical to SEQ ID NO: 131

SEQ ID NO: 222 - protein, 122 aa, Homo sapiens; identical to SEQ ID NO: 132

SEQ ID NO: 223 - protein, 5 aa, Homo sapiens
NYGLS

SEQ ID NO: 224 - protein, 17 aa, Homo sapiens
WINYDGGNTQYGQEFQG

SEQ ID NO: 225 - protein, 13 aa, Homo sapiens
DSSSSWARWFFDL

SEQ ID NO: 226 - DNA, 324 bp, Homo sapiens
tcctatgtgctgactcagccaccctcggtgtcagtggccccaggtaagacggccaggattccctgtgggggaaacttgatcgcggcccgcctcgtccattggtatcagcagaagccaggccaggcccctgtgctcatcatgttcgatgaccaggaccggccctcagggatccctgagcgattctctggctccaactctgggaacacggccaccctgaccatcagcagggtcgagaccggggatgaggccgactattattgtcaggtgtgggatactgggagtttgccggtggtattcggcggagggaccaagctgaccgtccta

SEQ ID NO: 227 - protein, 108 aa, Homo sapiens
SYVLTQPPSVSVAPGKTARIPCGGNLIAARLVHWYQQKPGQAPVLIMFDDQDRPSGIPERFSGSNSGNTATLTISRVETGDEADYYCQVWDTGSLPVVFGGGTKLTVL

SEQ ID NO: 228 - protein, 11 aa, Homo sapiens
GGNLIAARLVH

SEQ ID NO: 229 - protein, 7 aa, Homo sapiens
DDQDRPS

SEQ ID NO: 230 - protein, 11 aa, Homo sapiens
QVWDTGSLPVV

SEQ ID NO: 231 - DNA, 366 bp, Homo sapiens
caggtgcagctggtgcagtctggggctgaggtgaagaagcctggggcctcagtgaaggtctcctgcaaggcttctggttacaacgtcaactagacgggtctcagctgggtgcgacaggcccctggacaagggcttgagtggatgggatggatcaactacgacggcggcaacacacagtatggacaggaattccagggcagagtcaccatgaccacagatacatccacgagcacagcctacatggagttgaggagcctgagatctgacgacacggccgtttattactgtgcgagagactccagcagcagctgggcccgctggtttttcgatctctggggccgggggacactggtcaccgtctcgagt

SEQ ID NO: 232 - protein, 122 aa, Homo sapiens
QVQLVQSGAEVKKPGASVKVSCKASGYNVNQTGLSWVRQAPGQGLEWMGWINYDGGNTQYGQEFQGRVTMTTDTSTSTAYMELRSLRSDDTAVYYCARDSSSSWARWFFDLWGRGTLVTVSS

SEQ ID NO: 233 - protein, 5 aa, Homo sapiens
QTGLS

SEQ ID NO: 234 - protein, 17 aa, Homo sapiens
WINYDGGNTQYGQEFQG

SEQ ID NO: 235 - protein, 13 aa, Homo sapiens
DSSSSWARWFFDL

SEQ ID NO: 236 - DNA, 324 bp, Homo sapiens
tcctatgtgctgactcagccaccctcggtgtcagtggccccaggtaagacggccaggattccctgtgggggaaacatcattggaagtaaacttgtacactggtaccagcagaagccaggccaggcccctgtgttgatcgtgttcgatgacgaggaccggccctcagggatccctgagcgattctctggctccaactctgggaacacggccaccctgaccatcagcagggtcgaggccggggatgaggccgactattattgtcaggtgtgggatactggtagtgatcccgtggtattcggcggagggaccaagctgaccgtccta

SEQ ID NO: 237 - protein, 108 aa, Homo sapiens
SYVLTQPPSVSVAPGKTARIPCGGNIIGSKLVHWYQQKPGQAPVLIVFDDEDRPSGIPERFSGSNSGNTATLTISRVEAGDEADYYCQVWDTGSDPVVFGGGTKLTVL

SEQ ID NO: 238 - protein, 11 aa, Homo sapiens
GGNIIGSKLVH

SEQ ID NO: 239 - protein, 7 aa, Homo sapiens
DDEDRPS

SEQ ID NO: 240 - protein, 11 aa, Homo sapiens
QVWDTGSDPVV

SEQ ID NO: 241 - DNA, 366 bp, Homo sapiens; identical to SEQ ID NO: 1

SEQ ID NO: 242 - protein, 122 aa, Homo sapiens; identical to SEQ ID NO: 2

SEQ ID NO: 243 - protein, 5 aa, Homo sapiens
NYGLS

SEQ ID NO: 244 - protein, 17 aa, Homo sapiens
WINYDGGNTQYGQEFQG

SEQ ID NO: 245 - protein, 13 aa, Homo sapiens
DSSSSWARWFFDL

SEQ ID NO: 246 - DNA, 324 bp, Homo sapiens
tcctatgtgctgactcagccaccctcggtgtcagtggccccaggtaagacggccaggattccctgtgggggaaacatggtcggggcccgcctcgtccactggtaccagcagaagccaggccaggcccctgtgctggtcatctatgatgatggcgaccggccctcagggatccctgagcgattctctggctccaactctgggaacacggccaccctgaccatcagcagggtcgaggccggggatgaggccgactattattgtcaggtgtgggatactggtagtgatcccgtggtattcggcggagggaccaagctgaccgtccta

SEQ ID NO: 247 - protein, 108 aa, Homo sapiens
SYVLTQPPSVSVAPGKTARIPCGGNMVGARLVHWYQQKPGQAPVLVIYDDGDRPSGIPERFSGSNSGNTATLTISRVEAGDEADYYCQVWDTGSDPVVFGGGTKLTVL

SEQ ID NO: 248 - protein, 11 aa, Homo sapiens
GGNMVGARLVH

SEQ ID NO: 249 - protein, 7 aa, Homo sapiens
DDGDRPS

SEQ ID NO: 250 - protein, 11 aa, Homo sapiens
QVWDTGSDPVV

SEQ ID NO: 251 - protein, 11 aa, Artificial Sequence
  Description of Artificial Sequence Synthetic LCDR1 peptide
GGNXXXXXLVH

SEQ ID NO: 252 - protein, 7 aa, Artificial Sequence
  Description of Artificial Sequence Synthetic LCDR2 peptide
DDXDRPS

SEQ ID NO: 253 - protein, 11 aa, Artificial Sequence
  Description of Artificial Sequence Synthetic LCDR3 peptide
QVWDTGSXPVV

SEQ ID NO: 254 - protein, 30 aa, Artificial Sequence
  Description of Artificial Sequence Synthetic HFW1 polypeptide
QXQLVQSGAEVKKPGASVKVSCKASGYTFT

SEQ ID NO: 255 - protein, 14 aa, Artificial Sequence
  Description of Artificial Sequence Synthetic HFW2 peptide
WVRQAPGQGLEWXG

SEQ ID NO: 256 - protein, 32 aa, Artificial Sequence
  Description of Artificial Sequence Synthetic HFW3 polypeptide
RVTMTTDTSTXTAYMELRXLRSDDTAVYYCAR

SEQ ID NO: 257 - protein, 11 aa, Artificial Sequence
  Description of Artificial Sequence Synthetic HFW4 peptide
WGRGTLVTVSS

SEQ ID NO: 258 - protein, 22 aa, Artificial Sequence
  Description of Artificial Sequence Synthetic LFW1 peptide
SYVLTQPPXVSVAPGKTARIPC

SEQ ID NO: 259 - protein, 15 aa, Artificial Sequence
  Description of Artificial Sequence Synthetic LFW2 peptide
WYQQKPGQAPVLXXX

SEQ ID NO: 260 - protein, 32 aa, Artificial Sequence
  Description of Artificial Sequence Synthetic LFW3 polypeptide
GIPERFSGSNSGNTATLTISRVEXGDEADYYC

SEQ ID NO: 261 - protein, 10 aa, Artificial Sequence
  Description of Artificial Sequence Synthetic LFW4 peptide
FGGGTKLTVL

SEQ ID NO: 262 - protein, 30 aa, Artificial Sequence
  Description of Artificial Sequence Synthetic HFW1 polypeptide
QVQLVQSGAEVKKPGASVKVSCKASGYTFT

SEQ ID NO: 263 - protein, 14 aa, Artificial Sequence
  Description of Artificial Sequence Synthetic HFW2 peptide
WVRQAPGQGLEWMG

SEQ ID NO: 264 - protein, 32 aa, Artificial Sequence
  Description of Artificial Sequence Synthetic HFW3 polypeptide
RVTMTTDTSTSTAYMELRSLRSDDTAVYYCAR

SEQ ID NO: 265 - protein, 22 aa, Artificial Sequence
  Description of Artificial Sequence Synthetic LFW1 peptide
SYVLTQPPSVSVAPGKTARIPC

SEQ ID NO: 266 - protein, 15 aa, Artificial Sequence
  Description of Artificial Sequence Synthetic LFW2 peptide
WYQQKPGQAPVLIVF

SEQ ID NO: 267 - protein, 15 aa, Artificial Sequence
  Description of Artificial Sequence Synthetic LFW2 peptide
WYQQKPGQAPVLIIM

SEQ ID NO: 268 - protein, 15 aa, Artificial Sequence
  Description of Artificial Sequence Synthetic LFW2 peptide
WYQQKPGQAPVLIMF

SEQ ID NO: 269 - protein, 15 aa, Artificial Sequence
  Description of Artificial Sequence Synthetic LFW2 peptide
WYQQKPGQAPVLVIM

SEQ ID NO: 270 - protein, 15 aa, Artificial Sequence
  Description of Artificial Sequence Synthetic LFW2 peptide
WYQQKPGQAPVLIVY

SEQ ID NO: 271 - protein, 15 aa, Artificial Sequence
  Description of Artificial Sequence Synthetic LFW2 peptide
WYQQKPGQAPVLVIY

SEQ ID NO: 272 - protein, 32 aa, Artificial Sequence
  Description of Artificial Sequence Synthetic LFW2 polypeptide
GIPERFSGSNSGNTATLTISRVEAGDEADYYC

SEQ ID NO: 273 - protein, 27 aa, Homo sapiens
  human IL-Protein epitope sequence
DTKIEVAQFVKDLLLHLKKLFREGRFN

SEQ ID NO: 274 - protein, 34 aa, Homo sapiens
  human IL-Protein epitope sequence
FSSLHVRDTKIEVAQFVKDLLLHLKKLFREGRFN

SEQ ID NO: 275 - protein, 11 aa, Artificial Sequence
  Description of Artificial Sequence Synthetic LCDR1 peptide
GGNLLGARLVH

SEQ ID NO: 276 - protein, 107 aa, Artificial Sequence [sequence omitted]

SEQ ID NO: 277 - protein, 214 aa, Artificial Sequence
  Description of Artificial Sequence Synthetic Light Chain polypeptide of anti-IL5R
DIQMTQSPSSLSASVGDRVTITCGTSEDIINYLNWYQQKPGKAPKLLIYHTSRLQSGVPSRFSGSGSGTDFTLTISSLQPEDFATYYCQQGYTLPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 278 - protein, 121 aa, Artificial Sequence [sequence omitted]

SEQ ID NO: 279 - protein, 451 aa, Artificial Sequence
  Description of Artificial Sequence Synthetic Heavy Chain polypeptide of anti-IL5R
EVQLVQSGAEVKKPGASVKVSCKASGYTFTSYVIHWVRQRPGQGLAWMGYINPYNDGTKYNERFKGKVTITSDRSTSTVYMELSSLRSEDTAVYLCGREGIRYYGLLGDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 280 - protein, 5 aa, Artificial Sequence
  Description of Artificial Sequence Synthetic VH CDR1 peptide of anti-IL5R
SYVIH

SEQ ID NO: 281 - protein, 17 aa, Artificial Sequence
  Description of Artificial Sequence Synthetic VH CDR2 peptide of anti-IL5R
YINPYNDGTKYNERFKG

SEQ ID NO: 282 - protein, 12 aa, Artificial Sequence
  Description of Artificial Sequence Synthetic VH CDR3 peptide of anti-IL5R
EGIRYYGLLGDY

SEQ ID NO: 283 - protein, 11 aa, Artificial Sequence
  Description of Artificial Sequence Synthetic VL CDR1 peptide of anti-IL5R
GTSEDIINYLN

SEQ ID NO: 284 - protein, 7 aa, Artificial Sequence
  Description of Artificial Sequence Synthetic VL CDR2 peptide of anti-IL5R
HTSRLQS

SEQ ID NO: 285 - protein, 9 aa, Artificial Sequence
  Description of Artificial Sequence Synthetic VL CDR3 peptide of anti-IL5R
QQGYTLPYT

SEQ ID NO: 286 - protein, 4 aa, Artificial Sequence
  Description of Artificial Sequence Synthetic peptide
DDED

SEQ ID NO: 287 - protein, 4 aa, Artificial Sequence
  Description of Artificial Sequence Synthetic peptide
DNED

SEQ ID NO: 288 - protein, 4 aa, Artificial Sequence
  Description of Artificial Sequence Synthetic peptide
DDND

SEQ ID NO: 289 - protein, 4 aa, Artificial Sequence
  Description of Artificial Sequence Synthetic peptide
DDEN

SEQ ID NO: 290 - protein, 4 aa, Artificial Sequence
  Description of Artificial Sequence Synthetic peptide
ADEH

SEQ ID NO: 291 - protein, 4 aa, Artificial Sequence
  Description of Artificial Sequence Synthetic peptide
YDDQ

SEQ ID NO: 292 - protein, 4 aa, Artificial Sequence
  Description of Artificial Sequence Synthetic peptide
RDDQ

SEQ ID NO: 293 - protein, 4 aa, Artificial Sequence
  Description of Artificial Sequence Synthetic peptide
EDSG

SEQ ID NO: 294 - protein, 4 aa, Artificial Sequence
  Description of Artificial Sequence Synthetic peptide
EDTK

SEQ ID NO: 295 - protein, 4 aa, Artificial Sequence
  Description of Artificial Sequence Synthetic peptide
EDNQ

SEQ ID NO: 296 - protein, 4 aa, Artificial Sequence
  Description of Artificial Sequence Synthetic peptide
ADDE

SEQ ID NO: 297 - protein, 4 aa, Artificial Sequence
  Description of Artificial Sequence Synthetic peptide
YDDD

SEQ ID NO: 298 - protein, 4 aa, Artificial Sequence
  Description of Artificial Sequence Synthetic peptide
DDSD

SEQ ID NO: 299 - protein, 4 aa, Artificial Sequence
  Description of Artificial Sequence Synthetic peptide
EDSE

SEQ ID NO: 300 - protein, 14 aa, Artificial Sequence
  Description of Artificial Sequence Synthetic peptide
CGGNIIGSKLVHWY

SEQ ID NO: 301 - protein, 14 aa, Artificial Sequence
  Description of Artificial Sequence Synthetic peptide
VLVIYDDGDRPSGI

SEQ ID NO: 302 - protein, 17 aa, Artificial Sequence
  Description of Artificial Sequence Synthetic peptide
YYCQVWDTGSDPVVFGG

SEQ ID NO: 303 - protein, 14 aa, Artificial Sequence
  Description of Artificial Sequence Synthetic peptide
CGGNLIGARLVHWY

SEQ ID NO: 304 - protein, 14 aa, Artificial Sequence
  Description of Artificial Sequence Synthetic peptide
VLIIMDDIDRPSGI

SEQ ID NO: 305 - protein, 17 aa, Artificial Sequence
  Description of Artificial Sequence Synthetic peptide
YYCQVWDTGSDPVVFGG

SEQ ID NO: 306 - protein, 14 aa, Artificial Sequence
  Description of Artificial Sequence Synthetic peptide
CGGNMVGARLVHWY

SEQ ID NO: 307 - protein, 14 aa, Artificial Sequence
  Description of Artificial Sequence Synthetic peptide
VLIIMDDIDRPSGI

SEQ ID NO: 308 - protein, 17 aa, Artificial Sequence
  Description of Artificial Sequence Synthetic peptide
YYCQVWDTGSSPVVFGG

SEQ ID NO: 309 - protein, 14 aa, Artificial Sequence
  Description of Artificial Sequence Synthetic peptide
CGGNMVGARLVHWY

SEQ ID NO: 310 - protein, 14 aa, Artificial Sequence
  Description of Artificial Sequence Synthetic peptide
VLIIMDDIDRPSGI

SEQ ID NO: 311 - protein, 17 aa, Artificial Sequence
  Description of Artificial Sequence Synthetic peptide
YYCQVWDTGSRPVVFGG

SEQ ID NO: 312 - protein, 14 aa, Artificial Sequence
  Description of Artificial Sequence Synthetic peptide
CGGNMVGARLVHWY

SEQ ID NO: 313 - protein, 14 aa, Artificial Sequence
  Description of Artificial Sequence Synthetic peptide
VLVIMDDIDRPSGI

SEQ ID NO: 314 - protein, 17 aa, Artificial Sequence
  Description of Artificial Sequence Synthetic peptide
YYCQVWDTGSRPVVFGG

SEQ ID NO: 315 - protein, 14 aa, Artificial Sequence
  Description of Artificial Sequence Synthetic peptide
CGGNLIGARLVHWY

SEQ ID NO: 316 - protein, 14 aa, Artificial Sequence
  Description of Artificial Sequence Synthetic peptide
VLTIMDDIDRPSGI

SEQ ID NO: 317 - protein, 17 aa, Artificial Sequence
  Description of Artificial Sequence Synthetic peptide
YYCQVWDTGSRPVVFGG

SEQ ID NO: 318 - protein, 14 aa, Artificial Sequence
  Description of Artificial Sequence Synthetic peptide
CGGNMVGARLVHWY

SEQ ID NO: 319 - protein, 14 aa, Artificial Sequence
  Description of Artificial Sequence Synthetic peptide
VLTVFDDEDRPSGI

SEQ ID NO: 320 - protein, 17 aa, Artificial Sequence
  Description of Artificial Sequence Synthetic peptide
YYCQVWDTGSRPVVFGG

SEQ ID NO: 321 - protein, 14 aa, Artificial Sequence
  Description of Artificial Sequence Synthetic peptide
CGGNMVGARLVHWY

SEQ ID NO: 322 - protein, 14 aa, Artificial Sequence
  Description of Artificial Sequence Synthetic peptide
VLVIMDDIDRPSGI

SEQ ID NO: 323 - protein, 17 aa, Artificial Sequence
  Description of Artificial Sequence Synthetic peptide
YYCQVWDTGSRPVVFGG

SEQ ID NO: 324 - protein, 14 aa, Artificial Sequence
  Description of Artificial Sequence Synthetic peptide
CGGNMVGAYLVHWY

SEQ ID NO: 325 - protein, 14 aa, Artificial Sequence
  Description of Artificial Sequence Synthetic peptide
VLIVYDDMDRPSGI

SEQ ID NO: 326 - protein, 17 aa, Artificial Sequence
  Description of Artificial Sequence Synthetic peptide
YYCQVWDTGSSPVVFGG

SEQ ID NO: 327 - protein, 14 aa, Artificial Sequence
  Description of Artificial Sequence Synthetic peptide
CGGNMVGARLVHWY

SEQ ID NO: 328 - protein, 14 aa, Artificial Sequence
  Description of Artificial Sequence Synthetic peptide
VLIVFDDEDRPSGI

SEQ ID NO: 329 - protein, 17 aa, Artificial Sequence
  Description of Artificial Sequence Synthetic peptide
YYCQVWDTGSLPVVFGG

SEQ ID NO: 330 - protein, 14 aa, Artificial Sequence
  Description of Artificial Sequence Synthetic peptide
CGGNLIGARLVHWY

SEQ ID NO: 331 - protein, 14 aa, Artificial Sequence
  Description of Artificial Sequence Synthetic peptide
VLIMFDDQDRPSGI

SEQ ID NO: 332 - protein, 17 aa, Artificial Sequence
  Description of Artificial Sequence Synthetic peptide
YYCQVWDTGSLPVVFGG

SEQ ID NO: 333 - protein, 14 aa, Artificial Sequence
  Description of Artificial Sequence Synthetic peptide
CGGNMVGARLVHWY

SEQ ID NO: 334 - protein, 14 aa, Artificial Sequence
  Description of Artificial Sequence Synthetic peptide
VLIVYDDMDRPSGI

SEQ ID NO: 335 - protein, 17 aa, Artificial Sequence
  Description of Artificial Sequence Synthetic peptide
YYCQVWDTGSRPVVFGG

SEQ ID NO: 336 - protein, 14 aa, Artificial Sequence
  Description of Artificial Sequence Synthetic peptide
CGGNIIGSKLVHWY

SEQ ID NO: 337 - protein, 14 aa, Artificial Sequence
  Description of Artificial Sequence Synthetic peptide
VLVIYDDGDRPSGI

SEQ ID NO: 338 - protein, 16 aa, Artificial Sequence
  Description of Artificial Sequence Synthetic peptide
YYCQVWDTGSDPVVFG

SEQ ID NO: 339 - protein, 14 aa, Artificial Sequence
  Description of Artificial Sequence Synthetic peptide
CGGNLIGARLVHWY

SEQ ID NO: 340 - protein, 14 aa, Artificial Sequence
  Description of Artificial Sequence Synthetic peptide
VLVIMDDIDRPSGI

SEQ ID NO: 341 - protein, 16 aa, Artificial Sequence
  Description of Artificial Sequence Synthetic peptide
YYCQVWDTGSRPVVFG

SEQ ID NO: 342 - protein, 14 aa, Artificial Sequence
  Description of Artificial Sequence Synthetic peptide
CGGNMVAARLVHWY

SEQ ID NO: 343 - protein, 14 aa, Artificial Sequence
  Description of Artificial Sequence Synthetic peptide
VLIMFDDQDRPSGI

SEQ ID NO: 344 - protein, 16 aa, Artificial Sequence
  Description of Artificial Sequence Synthetic peptide
YYCQVWDTGSLPVVFG

SEQ ID NO: 345 - protein, 14 aa, Artificial Sequence
  Description of Artificial Sequence Synthetic peptide
CGGNLIAARLVHWY

SEQ ID NO: 346 - protein, 14 aa, Artificial Sequence
  Description of Artificial Sequence Synthetic peptide
VLIMFDDQDRPSGI

SEQ ID NO: 347 - protein, 16 aa, Artificial Sequence
  Description of Artificial Sequence Synthetic peptide
YYCQVWDTGSLPVVFG

SEQ ID NO: 348 - protein, 14 aa, Artificial Sequence
  Description of Artificial Sequence Synthetic peptide
CGGNLIGARLVHWY

SEQ ID NO: 349 - protein, 14 aa, Artificial Sequence
  Description of Artificial Sequence Synthetic peptide
VLIVYDDMDRPSGI

SEQ ID NO: 350 - protein, 16 aa, Artificial Sequence
  Description of Artificial Sequence Synthetic peptide
YYCQVWDTGSLPVVFG

SEQ ID NO: 351 - protein, 14 aa, Artificial Sequence
  Description of Artificial Sequence Synthetic peptide
CGGNMVGARLVHWY

SEQ ID NO: 352 - protein, 14 aa, Artificial Sequence
  Description of Artificial Sequence Synthetic peptide
VLIVFDDEDRPSGI

SEQ ID NO: 353 - protein, 16 aa, Artificial Sequence
  Description of Artificial Sequence Synthetic peptide
YYCQVWDTGSSPVVFG

SEQ ID NO: 354 - protein, 14 aa, Artificial Sequence
  Description of Artificial Sequence Synthetic peptide
CGGNLIGARLVHWY

SEQ ID NO: 355 - protein, 14 aa, Artificial Sequence
  Description of Artificial Sequence Synthetic peptide
VLIVYDDMDRPSGI

SEQ ID NO: 356 - protein, 16 aa, Artificial Sequence
  Description of Artificial Sequence Synthetic peptide
YYCQVWDTGSRPVVFG

SEQ ID NO: 357 - protein, 14 aa, Artificial Sequence
  Description of Artificial Sequence Synthetic peptide
CGGNLIAARLVHWY

SEQ ID NO: 358 - protein, 14 aa, Artificial Sequence
  Description of Artificial Sequence Synthetic peptide
VLIVFDDEDRPSGI

SEQ ID NO: 359 - protein, 16 aa, Artificial Sequence
  Description of Artificial Sequence Synthetic peptide
YYCQVWDTGSSPVVFG

SEQ ID NO: 360 - protein, 14 aa, Artificial Sequence
  Description of Artificial Sequence Synthetic peptide
CGGNMVGARLVHWY

SEQ ID NO: 361 - protein, 14 aa, Artificial Sequence
  Description of Artificial Sequence Synthetic peptide
VLIIMDDIDRPSGI

SEQ ID NO: 362 - protein, 16 aa, Artificial Sequence
  Description of Artificial Sequence Synthetic peptide
YYCQVWDTGSRPVVFG

SEQ ID NO: 363 - protein, 14 aa, Artificial Sequence
  Description of Artificial Sequence Synthetic peptide
CGGNMVGARLVHWY

SEQ ID NO: 364 - protein, 14 aa, Artificial Sequence
  Description of Artificial Sequence Synthetic peptide
VLIVFDDEDRPSGI

SEQ ID NO: 365 - protein, 16 aa, Artificial Sequence
  Description of Artificial Sequence Synthetic peptide
YYCQVWDTGSRPVVFG

SEQ ID NO: 366 - protein, 14 aa, Artificial Sequence
  Description of Artificial Sequence Synthetic peptide
CGGNLIAARLVHWY

SEQ ID NO: 367 - protein, 14 aa, Artificial Sequence
  Description of Artificial Sequence Synthetic peptide
VLIVFDDEDRPSGI

SEQ ID NO: 368 - protein, 16 aa, Artificial Sequence
  Description of Artificial Sequence Synthetic peptide
YYCQVWDTGSSPVVFG

SEQ ID NO: 369 - protein, 14 aa, Artificial Sequence
  Description of Artificial Sequence Synthetic peptide
CGGNMVGARLVHWY

SEQ ID NO: 370 - protein, 14 aa, Artificial Sequence
  Description of Artificial Sequence Synthetic peptide
VLIVYDDMDRPSGI

SEQ ID NO: 371 - protein, 16 aa, Artificial Sequence
  Description of Artificial Sequence Synthetic peptide
YYCQVWDTGSRPVVFG

SEQ ID NO: 372 - protein, 122 aa, Artificial Sequence; identical to SEQ ID NO: 2
  Description of Artificial Sequence Synthetic polypeptide

SEQ ID NO: 373 - protein, 122 aa, Artificial Sequence; identical to SEQ ID NO: 2
  Description of Artificial Sequence Synthetic polypeptide

SEQ ID NO: 374 - protein, 122 aa, Artificial Sequence; identical to SEQ ID NO: 42
  Description of Artificial Sequence Synthetic polypeptide

SEQ ID NO: 375 - protein, 122 aa, Artificial Sequence; identical to SEQ ID NO: 232
  Description of Artificial Sequence Synthetic polypeptide

SEQ ID NO: 376 - protein, 122 aa, Artificial Sequence; identical to SEQ ID NO: 2
  Description of Artificial Sequence Synthetic polypeptide

SEQ ID NO: 377 - protein, 122 aa, Artificial Sequence; identical to SEQ ID NO: 52
  Description of Artificial Sequence Synthetic polypeptide

SEQ ID NO: 378 - protein, 122 aa, Artificial Sequence; identical to SEQ ID NO: 2
  Description of Artificial Sequence Synthetic polypeptide

SEQ ID NO: 379 - protein, 122 aa, Artificial Sequence; identical to SEQ ID NO: 2
  Description of Artificial Sequence Synthetic polypeptide

SEQ ID NO: 380 - protein, 122 aa, Artificial Sequence; identical to SEQ ID NO: 152
  Description of Artificial Sequence Synthetic polypeptide

SEQ ID NO: 381 - protein, 122 aa, Artificial Sequence; identical to SEQ ID NO: 132
  Description of Artificial Sequence Synthetic polypeptide

SEQ ID NO: 382 - protein, 122 aa, Artificial Sequence; identical to SEQ ID NO: 2
  Description of Artificial Sequence Synthetic polypeptide

SEQ ID NO: 383 - protein, 122 aa, Artificial Sequence; identical to SEQ ID NO: 2
  Description of Artificial Sequence Synthetic polypeptide

SEQ ID NO: 384 - protein, 122 aa, Artificial Sequence; identical to SEQ ID NO: 2
  Description of Artificial Sequence Synthetic polypeptide

SEQ ID NO: 385 - protein, 122 aa, Artificial Sequence; identical to SEQ ID NO: 2
  Description of Artificial Sequence Synthetic polypeptide

SEQ ID NO: 386 - protein, 122 aa, Artificial Sequence; identical to SEQ ID NO: 2
  Description of Artificial Sequence Synthetic polypeptide

SEQ ID NO: 387 - protein, 122 aa, Artificial Sequence; identical to SEQ ID NO: 2
  Description of Artificial Sequence Synthetic polypeptide

SEQ ID NO: 388 - protein, 122 aa, Artificial Sequence; identical to SEQ ID NO: 2
  Description of Artificial Sequence Synthetic polypeptide

SEQ ID NO: 389 - protein, 122 aa, Artificial Sequence; identical to SEQ ID NO: 2
  Description of Artificial Sequence Synthetic polypeptide

SEQ ID NO: 390 - protein, 122 aa, Artificial Sequence; identical to SEQ ID NO: 2
  Description of Artificial Sequence Synthetic polypeptide

SEQ ID NO: 391 - protein, 122 aa, Artificial Sequence; identical to SEQ ID NO: 2
  Description of Artificial Sequence Synthetic polypeptide

SEQ ID NO: 392 - protein, 122 aa, Artificial Sequence; identical to SEQ ID NO: 2
  Description of Artificial Sequence Synthetic polypeptide

SEQ ID NO: 393 - protein, 122 aa, Artificial Sequence; identical to SEQ ID NO: 132
  Description of Artificial Sequence Synthetic polypeptide

SEQ ID NO: 394 - protein, 122 aa, Artificial Sequence; identical to SEQ ID NO: 2
  Description of Artificial Sequence Synthetic polypeptide

SEQ ID NO: 395 - protein, 122 aa, Artificial Sequence; identical to SEQ ID NO: 2
  Description of Artificial Sequence Synthetic polypeptide

SEQ ID NO: 396 - protein, 122 aa, Artificial Sequence; identical to SEQ ID NO: 2
  Description of Artificial Sequence Synthetic polypeptide

SEQ ID NO: 397 - protein, 108 aa, Artificial Sequence; identical to SEQ ID NO: 7
  Description of Artificial Sequence Synthetic polypeptide

SEQ ID NO: 398 - protein, 108 aa, Artificial Sequence; identical to SEQ ID NO: 247
  Description of Artificial Sequence Synthetic polypeptide

SEQ ID NO: 399 - protein, 108 aa, Artificial Sequence; identical to SEQ ID NO: 47
  Description of Artificial Sequence Synthetic polypeptide

SEQ ID NO: 400 - protein, 108 aa, Artificial Sequence; identical to SEQ ID NO: 237
  Description of Artificial Sequence Synthetic polypeptide

SEQ ID NO: 401 - protein, 108 aa, Artificial Sequence; identical to SEQ ID NO: 107
  Description of Artificial Sequence Synthetic polypeptide

SEQ ID NO: 402 - protein, 108 aa, Artificial Sequence; identical to SEQ ID NO: 57
  Description of Artificial Sequence Synthetic polypeptide

SEQ ID NO: 403 - protein, 108 aa, Artificial Sequence; identical to SEQ ID NO: 17
  Description of Artificial Sequence Synthetic polypeptide

SEQ ID NO: 404 - protein, 108 aa, Artificial Sequence; identical to SEQ ID NO: 187
  Description of Artificial Sequence Synthetic polypeptide

SEQ ID NO: 405 - protein, 108 aa, Artificial Sequence; identical to SEQ ID NO: 157
  Description of Artificial Sequence Synthetic polypeptide

SEQ ID NO: 406 - protein, 108 aa, Artificial Sequence; identical to SEQ ID NO: 137
  Description of Artificial Sequence Synthetic polypeptide

SEQ ID NO: 407 - protein, 108 aa, Artificial Sequence; identical to SEQ ID NO: 167
  Description of Artificial Sequence Synthetic polypeptide

SEQ ID NO: 408 - protein, 108 aa, Artificial Sequence; identical to SEQ ID NO: 127
  Description of Artificial Sequence Synthetic polypeptide

SEQ ID NO: 409 - protein, 108 aa, Artificial Sequence; identical to SEQ ID NO: 177
  Description of Artificial Sequence Synthetic polypeptide

SEQ ID NO: 410 - protein, 108 aa, Artificial Sequence; identical to SEQ ID NO: 27
  Description of Artificial Sequence Synthetic polypeptide

SEQ ID NO: 411 - protein, 108 aa, Artificial Sequence; identical to SEQ ID NO: 77
  Description of Artificial Sequence Synthetic polypeptide

SEQ ID NO: 412 - protein, 108 aa, Artificial Sequence; identical to SEQ ID NO: 77
  Description of Artificial Sequence Synthetic polypeptide

SEQ ID NO: 413 - protein, 108 aa, Artificial Sequence; identical to SEQ ID NO: 67
  Description of Artificial Sequence Synthetic polypeptide

SEQ ID NO: 414 - protein, 108 aa, Artificial Sequence; identical to SEQ ID NO: 67
  Description of Artificial Sequence Synthetic polypeptide

SEQ ID NO: 415 - protein, 108 aa, Artificial Sequence; identical to SEQ ID NO: 87
  Description of Artificial Sequence Synthetic polypeptide

SEQ ID NO: 416 - protein, 108 aa, Artificial Sequence; identical to SEQ ID NO: 87
  Description of Artificial Sequence Synthetic polypeptide

SEQ ID NO: 417 - protein, 108 aa, Artificial Sequence; identical to SEQ ID NO: 37
  Description of Artificial Sequence Synthetic polypeptide

SEQ ID NO: 418 - protein, 108 aa, Artificial Sequence; identical to SEQ ID NO: 227
  Description of Artificial Sequence Synthetic polypeptide

SEQ ID NO: 419 - protein, 108 aa, Artificial Sequence; identical to SEQ ID NO: 207
  Description of Artificial Sequence Synthetic polypeptide

SEQ ID NO: 420 - protein, 108 aa, Artificial Sequence; identical to SEQ ID NO: 147
  Description of Artificial Sequence Synthetic polypeptide

SEQ ID NO: 421 - protein, 108 aa, Artificial Sequence; identical to SEQ ID NO: 117
  Description of Artificial Sequence Synthetic polypeptide

SEQ ID NO: 422 - protein, 122 aa, Artificial Sequence; identical to SEQ ID NO: 2
  Description of Artificial Sequence Synthetic polypeptide

SEQ ID NO: 423 - protein, 122 aa, Artificial Sequence; identical to SEQ ID NO: 2
  Description of Artificial Sequence Synthetic polypeptide

SEQ ID NO: 424 - protein, 122 aa, Artificial Sequence; identical to SEQ ID NO: 2
  Description of Artificial Sequence Synthetic polypeptide

SEQ ID NO: 425 - protein, 122 aa, Artificial Sequence; identical to SEQ ID NO: 2
  Description of Artificial Sequence Synthetic polypeptide

SEQ ID NO: 426 - protein, 108 aa, Artificial Sequence
  Description of Artificial Sequence Synthetic polypeptide
SYVLTQPPSVSVAPGKTARITCGGNIIGSKLVHWYQQKPGQAPVLVIYDDGDRPSGIPERFSGSNSGNTATLTISRVEAGDEADYYCQVWDTGSDPVVFGGGTKLTVL

SEQ ID NO: 427 - protein, 108 aa, Artificial Sequence
  Description of Artificial Sequence Synthetic polypeptide
SYVLTQPPSVSVAPGKTARITCGGNLIGARLVHWYQQKPGQAPVLIIMDDIDRPSGIPERFSGSNSGNTATLTISRVEAGDEADYYCQVWDTGSRPVVFGGGTKLTVL

SEQ ID NO: 428 - protein, 108 aa, Artificial Sequence
  Description of Artificial Sequence Synthetic polypeptide
SYVLTQPPSVSVAPGKTARITCGGNLIGARLVHWYQQKPGQAPVLIMFDDQDRPSGIPERFSGSNSGNTATLTISRVEAGDEADYYCQVWDTGSLPVVFGGGTKLTVL

SEQ ID NO: 429 - protein, 108 aa, Artificial Sequence
  Description of Artificial Sequence Synthetic polypeptide
SYVLTQPPSVSVAPGKTARITCGGNMVGARLVHWYQQKPGQAPVLIVFDDEDRPSGIPERFSGSNSGNTATLTISRVEAGDEADYYCQVWDTGSSPVVFGGGTKLTVL

SEQ ID NO: 430 - protein, 108 aa, Artificial Sequence; identical to SEQ ID NO: 426
  Description of Artificial Sequence Synthetic polypeptide

SEQ ID NO: 431 - protein, 108 aa, Artificial Sequence
  Description of Artificial Sequence Synthetic polypeptide
SYVLTQPPSVSVAPGKTARITCGGNMVGARLVHWYQQKPGQAPVLVIYDDGDRPSGIPERFSGSNSGNTATLTISRVEAGDEADYYCQVWDTGSDPVVFGGGTKLTVL

SEQ ID NO: 432 - protein, 108 aa, Artificial Sequence
  Description of Artificial Sequence Synthetic polypeptide
SYVLTQPPSVSVAPGKTARITCGGNIIGSKLVHWYQQKPGQAPVLIVFDDEDRPSGIPERFSGSNSGNTATLTISRVEAGDEADYYCQVWDTGSDPVVFGGGTKLTVL

SEQ ID NO: 433 - protein, 108 aa, Artificial Sequence; identical to SEQ ID NO: 429
  Description of Artificial Sequence Synthetic polypeptide